SEQUENCE LISTING (41 sequences)

SEQ ID NO: 1 - DNA, 6603 bp, synthetic construct
  misc_feature 1..6603: note = Synthetic: nucleic acid sequence of the Tn7 vector
  source 1..6603: mol_type = other DNA
ttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattccacatgtggaattccacatgtggaattgtgagcggataacaatttgtggaattcccgggagagctcgatcggccgaagcaggggggcaaggctgaaaagccggcccccgctgcggccccgaccggcttcaccttcaacccaacaccggacaaaaaggatcctctagaggaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggggtcgaaattcccggggatccacgcgtcttaaggcggccttgcagtttcatttgatgctcgatgagtttttctaatcagaattggttaattggttgtaacacgcggccgctcggtgagaccgacttataaacgcagaaaggcccacccgaaggtgagccagtgtgactctagtagagagcgttcaccgacaaacaacagataaaacgaaaggcccagtctttcgactgagcctttcgttttatttgatgcctggagatccttattaagcaccggtggagtgacgaccttcagcacgttcgtactgttcaacgatggtgtagtcttcgttgtgggaggtgatgtccagtttgatgtcggttttgtaagcacccggcagctgaaccggttttttagccatgtaggtggttttaacttcagcgtcgtagtgaccaccgtctttcagtttcagacgcattttgatttcacctttcagagcaccgtcttccgggtacatacgttcggtggaagcttcccaacccatggtttttttctgcataaccggaccgtcggacgggaagttagtaccacgcagtttaactttgtagatgaactcaccgtcttgcagggaggagtcctgggtaacggtaacaacaccaccgtcttcgaagttcataacacgttcccatttgaaaccttccgggaaggacagtttcaggtagtccgggatgtcagccgggtgtttaacgtaagctttggaaccgtactggaactgcggggacaggatgtcccaagcgaacggcagcggaccacctttggtaactttcagtttagcggtctgagtaccttcgtacggacgaccttcaccttcaccttcgatttcgaactcgtgaccgttaacggaaccttccatacgaactttgaaacgcatgaactctttgataacgtcttcgctactcgccatagatcctttctcctctttcagatccgtgctcagtatctctatcactgatagggatgtcaatctctatcactgatagggactggtctcaaggggcctgtcagaccaagtttactcatatatactttagattgatttctgaaagcgaccaggtgctcgcggccgcggtaccgggcccgtcgggatccggtgattgattgagcaagctttatgcttgtaaaccgttttgtgaaaaaatttttaaaataaaaaaggggacctctagggtccccaattagaattggccgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaattcgtaattggggaccctagaggtccccttttttattttaaaaattttttcacaaaacggtttacaagcataaagcttgctcaatcaatcaccggatccccgactctagtcgacctgcaggccaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttgggcgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatcgcatgcggtacctctagaagaagcttgggatccgtcgacctgcagatctgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttcc

SEQ ID NO: 2 - DNA, 10710 bp, synthetic construct
  misc_feature 1..10710: note = Synthetic: nucleic acid sequence of the Tn10 vector
  source 1..10710: mol_type = other DNA
ctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccccaggctttacactttatgcttccgacctgcagatctgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctgggtcacgtgcgcgtcacgaactgcgaggcaaacaccctgcccgctgtcatggccgaggtgatggcgacccagcacggcaacacccgttccgaggccgacaagacctatcacctgctggttagcttccgcgcgggagagaagcccgacgcggagacgttgcgcgcgattgaggaccgcatctgcgctgggcttggcttcgccgagcatcagcgcgtcagtgccgtgcatcacgacaccgacaacctgcacatccatatcgccatcaacaagattcacccgacccgaaacaccatccatgagccgtatcgggcctaccgcgccctcgctgacctctgcgcgacgctcgaacgggactacgggcttgagcgtgacaatcacgaaacgcggcagcgcgtttccgagaaccgcgcgaacgacatggagcggcacgcgggcgtggaaagcctggtcggctggatccggccacgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagttatttcgtcaccaaatgatgttattccgcgaaatataatgaccctcttgataacccaagagggcattttttacgataaagaagatttagcttcaaataaaacctatctattttatttatctttcaagctcaataaaaagccgcggtaaatagcaataaattggccttttttatcggcaagctcttttaggtttttcgcatgtattgcgatatgcataaaccagccattgagtaagtttttaagcacatcatcatcataagctttcctgacggaatgttaattctcgttgaccctgagcactgatgaatcccctaatgattttggtaaaaatcattaagttaaggtggatacacatcttgtcatatgatcccggatccggctgtaatccgggcagcgcaacggaacattcatcagtgtaaaaatggaatcaataaagccctgcgcagcgcgcagggtcagcctgaatacgcgtggccgcctaggccgcggccgccgggcaagtacgacatcacccggcccaaggcggcaggctgactcacgttaagggattttggtcatgactgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacacgcggccgctcggtgagaccgacttataaacgcagaaaggcccacccgaaggtgagccagtgtgactctagtagagagcgttcaccgacaaacaacagataaaacgaaaggcccagtctttcgactgagcctttcgttttatttgatgcctggagatccttattaagcaccggtggagtgacgaccttcagcacgttcgtactgttcaacgatggtgtagtcttcgttgtgggaggtgatgtccagtttgatgtcggttttgtaagcacccggcagctgaaccggttttttagccatgtaggtggttttaacttcagcgtcgtagtgaccaccgtctttcagtttcagacgcattttgatttcacctttcagagcaccgtcttccgggtacatacgttcggtggaagcttcccaacccatggtttttttctgcataaccggaccgtcggacgggaagttagtaccacgcagtttaactttgtagatgaactcaccgtcttgcagggaggagtcctgggtaacggtaacaacaccaccgtcttcgaagttcataacacgttcccatttgaaaccttccgggaaggacagtttcaggtagtccgggatgtcagccgggtgtttaacgtaagctttggaaccgtactggaactgcggggacaggatgtcccaagcgaacggcagcggaccacctttggtaactttcagtttagcggtctgagtaccttcgtacggacgaccttcaccttcaccttcgatttcgaactcgtgaccgttaacggaaccttccatacgaactttgaaacgcatgaactctttgataacgtcttcgctactcgccatagatcctttctcctctttcagatccgtgctcagtatctctatcactgatagggatgtcaatctctatcactgatagggactggtctcaaggggcctgtcagaccaagtttactcatatatactttagattgatttctgaaagcgaccaggtgctcgcggccgcacgcgtattcaggctgaccctgcgcgctgcgcagggctttattgattccatttttacactgatgaatgttccgttgcgctgcccggattacagccggatccgggatcatatgacaagatgtgtatccaccttaacttaatgatttttaccaaaatcattaggggattcatcagtgctcagggtcaacgagaattaacattccgtcaggaaagctttaagttggttctcttggatcaatttgctgacaatggcgtttaccttaccagtaatgtattcaaggctaattttttcaagttcattccaaccaatgataggcatcacttcttggatagggataaggtttttattattatcaataatataatcaagataatgttcaaatatactttctaaggcagaccaaccatttgttaaatcagtttttgttgtgatgtaggcatcaatcataattaattgctgcttataacaggcactgagtaattgttttttatttttaaagtgatgataaaaggcacctttggtcaccaacgcttttcccgagatcgatctcatctattgaaacagcttgatagcctttttcaacaaacaatattcgtgctgagttaaccagtgattgataggtactcttaaaattttcttgttgatgatttttattttccatgatagatttaaaataacataccgtcagtatgtttatggtatcatgatgatgtggtcgtgacaatcttaagaacatttaggttattttatgtatattgaacagcattctcgctatcaaaataaagctaataacatccaattaagatatgatgataagcagtttcatacaacggttatcaaagatgttctattatggattgaacataatttagatcagtctttactgcttgatgatgtggcgaataaagcgggttataccaagtggtattttcagcggctgttcaaaaagtaacaggggtcacactggctagctatattcgtgctcgtcgtttgacgaaagcggctgttgagttgaggttgacgaaaaaaactatccttgagatcgcattaaaatatcaatttgattcccaacaatcttttacacgtcgatttaagtacatttttaaggttacaccaagttattatcggcgtaataaattatgggaattggaggcaatgcactgagagatcccctcataatttccccaaagcgtaaccatgtgtgaataaattttgagctagtagggttgcagccacgagtaagtcttcccttgttattgtgtagccagaatgccgcaaaacttccatgcctaagcgaactgttgagagtacgtttcgatttctgactgtgttagcctggaagtgcttgtcccaaccttgtttctgagcatgaacgcccgcaagccaacatgttagttgaagcatcagggcgattagcagcatgatatcaaaacgctctgagctgctcgttcggctatggcgtaggcctagtccgtaggcaggacttttcaagtctcggaaggtttcttcaatctgcattcgcttcgaatagatattaacaagttgtttgggtgttcgaatttcaacaggtaagttagttgctagaacccatggctcctttgccgacgctgagtagattttaggtgacgggtggtgacaatgagtccgtgtcgagcgctgattttttcggcctttagagcgagatttatacaatagaatttggcatgagattggattgcttttagtcagcctcttatagcctaaagtctttgagtgactagatgacatatcatgtaagttgctgataggtttccagttttccgctcctaggtctgcatattgtacttttcctcttactcgacttaaccagtaccaacccagcttctcaacggatttataccatggcactttaaagccagcatcactgacaatgagcggtgtggtgttactcggtagaatgctcgcaaggtcggctagaaattggtcatgagctttctttgaacattgctctgaaagcgggaacgctttctcataaagagtaacagaacgaccgtgtagtgcgactgaagctcgcaataccataagtcgtttttgctcacgaatatcagaccagtcaacaagtacaatgggcatcgtattgcccgaacagataaagctagcatgccaacggtatacagcgagtcgctctttgtggaggtgacgattacctaacaatcggtcgattcgtttgatgttatgttttgttctcgctttggttggcaggttacggccaagttcggtaagagtgagagttttacagtcaagtaatgcgtggcaagccaacgttaagctgttgagtcgttttaagtgtaattcggggcagaattggtaaagagagtcgtgtaaaatatcgagttcgcacatcttgttgtctgattattgatttttcgcgaaaccatttgatcctgtttcctgtgtgaaattgttatccgctcacaattccacacattatacgagccgatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgatgataagctgtcaaacatgagaattcccgggagagctcggtacccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgc

SEQ ID NO: 3 - DNA, 1835 bp, synthetic construct
  misc_feature 1..1835: note = Synthetic: exemplary Type-1 linker vector
  source 1..1835: mol_type = other DNA
tcggtctcaccctgaattcgcatctagactgatgagacgtggtagagccacaaacagccggtacaagcaacgatctccaggaccatctgaatcatgcgcggatgacacgaactcacgacggcgatcacagacattaacccacagtacagacactgcgacaacgtggcaattcgtcgcaatacaacgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtag

SEQ ID NO: 4 - DNA, 1750 bp, synthetic construct
  misc_feature 1..1750: note = Synthetic: exemplary Type-2 CP25 promoter vector
  source 1..1750: mol_type = other DNA
gtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcaaacgctttggcagtttattcttgacatgtagtgagggggctggtataatcacatagtactgttatacagaaacagaggagatattacatatgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaac

SEQ ID NO: 5 - DNA, 2000 bp, synthetic construct
  misc_feature 1..2000: note = Synthetic: exemplary Type-2 macB promoter vector
  source 1..2000: mol_type = other DNA
gtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcaaacggaagtttctgcggtgcttttaaactatgagccgagaattatccttgaggatatttcgtttgatatgtcagatgtgtacgaaggtgccttattaatagagctcacttatttgatccgcaaaaccaatagccgcagcaatatggtgtttccgttttatctcgccgagcagtctgtttaaacccgcgtttcaaacatttggtatcacccacagaatcacccatttcgaccttgctcgccaagcaatatgcagacccgcaccgcttatccttttaccaaggttaaattgacctagtagtgcataggtatgctcattaataacattaataaggtaatcttatgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaac

SEQ ID NO: 6 - DNA, 2014 bp, synthetic construct
  misc_feature 1..2014: note = Synthetic: exemplary Type-2 bmp1 promoter vector
  source 1..2014: mol_type = other DNA
gtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcaaacgcgaaccaccacattctccttcaataccttccactaggttttctattgcagcctgcataatagacatacctggctccacttcggtctcgatggtttcaccgtcatgctgaataaatgtgatttttagcatagtaactccattattaatttttaaaataagaactaataaattacctgttagtttgttttttcatatagtccgatgtaactaattttatagtcccacttggctattttattgttttaatatttctatattgttttatttgaattttaatctaatggagttttaaaaggtggaaaaagaattaatagactttataaataacgatctactcgaaggtgctgctatgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaac

SEQ ID NO: 7 - DNA, 2380 bp, synthetic construct
  misc_feature 1..2380: note = Synthetic: exemplary Type-3 coding GFP vector
  source 1..2380: mol_type = other DNA
tcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcatatgagtaaaggagaagagcttttcacaggagttgtcccaatcctcgtggaattagacggtgatgttaatgggcacaagttctctgtcagtggagagggtgaaggcgacgcaacatatggcaagctgacccttaaatttatttgcaccacgggtaaactacctgttccatggccaacactggtcactacgttcgggtatggggttcagtgctttgcgcgctacccagatcacatgaaacagcacgactttttcaagagtgcaatgcccgaaggctatgtacaggagagaaccatcttttttaaggatgacggcaactataagacacgcgccgaagtgaagttcgagggtgatacccttgttaatagaatcgagttaaagggtattgactttaaggaagatggaaatattttaggccacaaactggaatataactataactcccataatgtgtacattatggccgacaagcaaaagaacggtatcaaggttaacttcaagatcagacacaacattgaggatggaagcgttcaactagccgaccattaccaacaaaacaccccaattggcgatgggcctgtgctgttaccagacaaccattacctgtccactcaatctgccctttcgaaagatcccaacgaaaagcgcgaccacatggtccttcttgagtttgtcacggctgctgggattacacacggcatggatgaactatacaaataaatcctgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaa

SEQ ID NO: 8 - DNA, 2179 bp, synthetic construct
  misc_feature 1..2179: note = Synthetic: exemplary Type-3 coding nanoluc vector
  source 1..2179: mol_type = other DNA
tcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcatatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaatcctgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaa

SEQ ID NO: 9 - DNA, 2376 bp, synthetic construct
  misc_feature 1..2376: note = Synthetic: exemplary Type-3 coding mRuby vector
  source 1..2376: mol_type = other DNA
tcggtctcatatggtgtccaaaggagaggagttaatcaaggaaaacatgagaatgaaagttgtcatggagggctccgttaatggtcaccaattcaagtgtacaggggaaggtgaaggtaatccttacatgggtacacaaactatgagaattaaagtaattgaaggcggaccactaccatttgcatttgacattctggcaacgtcattcatgtacggatcacgaactttcatcaagtaccctaaaggtataccagactttttcaagcaatcttttccagagggttttacatgggaaagggttacaagatacgaagatgggggtgtcgtcacagttatgcaagatacttcattagaagatggctgccttgtctatcatgtgcaagtaagaggggtgaattttccttctaacggacctgtgatgcagaaaaagaccaaaggttgggaaccaaatactgaaatgatgtacccagctgatggaggtttgagaggctacacacacatggcgcttaaagttgatggtggaggtcatttgtcttgtagttttgttaccacttatcgttctaaaaagactgttggcaatatcaaaatgccaggaatacatgctgtagaccacagactagaaagactcgaagagagcgataacgaaatgttcgttgtacagagagagcatgccgtagccaaatttgctggcttaggcggtggtatggatgaattgtataagggatcctgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtag

SEQ ID NO: 10 - DNA, 1720 bp, synthetic construct
  misc_feature 1..1720: note = Synthetic: exemplary Type-4 terminator vector
  source 1..1720: mol_type = other DNA
ggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcaatcctaactagcataaccccttggggcctctaaacgggtcttgaggggttttttgctggctgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagc

SEQ ID NO: 11 - DNA, 1856 bp, synthetic construct
  misc_feature 1..1856: note = Synthetic: exemplary Type-5 linker vector
  source 1..1856: mol_type = other DNA
tcggtctcagctggaaatctgctcgtcagtggtgctcacactgacgaatcatgtacagatcataccgatgactgcctggcgactcacaactaagcaagacagccggaaccagcgccggcgaacaccactgcatatatggcatatcacaacagtccacgtctcaagcagttacagagatgttacgaaccactagtgcactgcagtacatgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtag

SEQ ID NO: 12 - DNA, 7608 bp, synthetic construct
  misc_feature 1..7608: note = Synthetic: exemplary Type-8 RSF1010 backbone vector
  source 1..7608: mol_type = other DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagtcatgaccaaaatcccttaacgtgagtcagcctgccgccttgggccgggtgatgtcgtacttgcccgccgcgaactcggttaccgtccagcccagcgcgaccagctccggcaacgcctcgcgcacccgcttgcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatgaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtgggtgcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgcttggtctggctcatgacctgccgggcttcgtcggccagtgtcgccatgctctgggccagcggttcgatctgctccgctaactcgttgatgcctctggatttcttcactctgtcgattgcgttcatggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggagcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagaaatcaatctaaagtatatatgagtaaacttggtctgacaggccccttgagaccagtccctatcagtgatagagattgacatccctatcagtgatagagatactgagcacggatctgaaagaggagaaaggatctatggcgagtagcgaagacgttatcaaagagttcatgcgtttcaaagttcgtatggaaggttccgttaacggtcacgagttcgaaatcgaaggtgaaggtgaaggtcgtccgtacgaaggtactcagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtactaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaataaggatctccaggcatcaaataaaacgaaaggctcagtcgaaagactgggcctttcgttttatctgttgtttgtcggtgaacgctctctactagagtcacactggctcaccttcgggtgggcctttctgcgtttataagtcggtctcaccgagcggccgcgtgttacaaccaattaaccaattctga

SEQ ID NO: 13 - DNA, 24 bp, synthetic construct
  misc_feature 1..24: note = Synthetic: p107_bbamp_F
  source 1..24: mol_type = other DNA
tatgtgagaccagaccaataaaaa

SEQ ID NO: 14 - DNA, 22 bp, synthetic construct
  misc_feature 1..22: note = Synthetic: p107_bbamp_R
  source 1..22: mol_type = other DNA
cgtttgagaccgactacggtta

SEQ ID NO: 15 - DNA, 23 bp, synthetic construct
  misc_feature 1..23: note = Synthetic: macb_seq_f
  source 1..23: mol_type = other DNA
atgagccgagaattatccttgag

SEQ ID NO: 16 - DNA, 20 bp, synthetic construct
  misc_feature 1..20: note = Synthetic: sheath_seq_f
  source 1..20: mol_type = other DNA
catggcgtcatagcagtaca

SEQ ID NO: 17 - DNA, 42 bp, synthetic construct
  misc_feature 1..42: note = Synthetic: ptac_gbsn_F2
  source 1..42: mol_type = other DNA
taaccgtagtcggtctcaaacggcactcccgttctggataat

SEQ ID NO: 18 - DNA, 44 bp, synthetic construct
  misc_feature 1..44: note = Synthetic: ptac_gbsn_R2
  source 1..44: mol_type = other DNA
tttttattggtctggtctcacatagggacaactccagtgaaaag

SEQ ID NO: 19 - DNA, 40 bp, synthetic construct
  misc_feature 1..40: note = Synthetic: pBTK615_ptac_macB1_sgRNA_F
  source 1..40: mol_type = other DNA
tcggctcgtataatgtgtggaagctcggggatctgtcgtg

SEQ ID NO: 20 - DNA, 49 bp, synthetic construct
  misc_feature 1..49: note = Synthetic: pBTK615_ptac_macB1_sgRNA_R
  source 1..49: mol_type = other DNA
ttttaacttgctatttctagctctaaaaccacgacagatccccgagctt

SEQ ID NO: 21 - DNA, 44 bp, synthetic construct
  misc_feature 1..44: note = Synthetic: pBTK107_macB_promoter_gbsn_F1
  source 1..44: mol_type = other DNA
gataaccgtagtcggtctcaaacggaagtttctgcggtgctttt

SEQ ID NO: 22 - DNA, 55 bp, synthetic construct
  misc_feature 1..55: note = Synthetic: pBTK107_macB_promoter_gbsn_R1
  source 1..55: mol_type = other DNA
tttttattggtctggtctcacataagattaccttattaatgttattaatgagcat

SEQ ID NO: 23 - DNA, 46 bp, synthetic construct
  misc_feature 1..46: note = Synthetic: pBTK107_sheath_promoter_gbsn_F1
  source 1..46: mol_type = other DNA
gataaccgtagtcggtctcaaacgacaccgactttaccctatctcg

SEQ ID NO: 24 - DNA, 52 bp, synthetic construct
  misc_feature 1..52: note = Synthetic: pBTK107_sheath_promoter_gbsn_R1
  source 1..52: mol_type = other DNA
tttttattggtctggtctcacatagtttttccttacgttgataattacattc

SEQ ID NO: 25 - DNA, 20 bp, synthetic construct
  misc_feature 1..20: note = Synthetic: pBTK107_CP25_F
  source 1..20: mol_type = other DNA
tgagggggctggtataatca

SEQ ID NO: 26 - DNA, 27 bp, synthetic construct
  misc_feature 1..27: note = Synthetic: gRNA_VioA5_F
  source 1..27: mol_type = other DNA
cacatatttatgttcataaactcgaag

SEQ ID NO: 27 - DNA, 20 bp, synthetic construct
  misc_feature 1..20: note = Synthetic: pBTK615_ptac_seqF1
  source 1..20: mol_type = other DNA
acagacactgcgacaacgtg

SEQ ID NO: 28 - DNA, 21 bp, synthetic construct
  misc_feature 1..21: note = Synthetic: pBTK615_gRNA_GFP
  source 1..21: mol_type = other DNA
cgtctaattccacgaggattg

SEQ ID NO: 29 - DNA, 40 bp, synthetic construct
  misc_feature 1..40: note = Synthetic: p615_ptac_gRNA_VioA5_F2
  source 1..40: mol_type = other DNA
tcggctcgtataatgtgtggtttatgttcataaactcgaa

SEQ ID NO: 30 - DNA, 49 bp, synthetic construct
  misc_feature 1..49: note = Synthetic: p615_ptac_gRNA_VioA5_R2
  source 1..49: mol_type = other DNA
ttttaacttgctatttctagctctaaaacttcgagtttatgaacataaa

SEQ ID NO: 31 - DNA, 20 bp, synthetic construct
  misc_feature 1..20: note = Synthetic: p615_ptac_vector_amplification_R
  source 1..20: mol_type = other DNA
ccacacattatacgagccga

SEQ ID NO: 32 - DNA, 29 bp, synthetic construct
  misc_feature 1..29: note = Synthetic: p615_vector_amplification_F
  source 1..29: mol_type = other DNA
gttttagagctagaaatagcaagttaaaa

SEQ ID NO: 33 - DNA, 51 bp, synthetic construct
  misc_feature 1..51: note = Synthetic: p615-ptac_F2
  source 1..51: mol_type = other DNA
caattaatcatcggctcgtataatgtgtggcgtctaattccacgaggattg

SEQ ID NO: 34 - DNA, 50 bp, synthetic construct
  misc_feature 1..50: note = Synthetic: p615-ptac_R2
  source 1..50: mol_type = other DNA
tacgagccgatgattaattgtcaacagctcttcagtgagacggtattgcg

SEQ ID NO: 35 - DNA, 20 bp, synthetic construct
  misc_feature 1..20: note = Synthetic: 61A2_Kan_intF2
  source 1..20: mol_type = other DNA
ctgcctcggtgagttttctc

SEQ ID NO: 36 - DNA, 54 bp, synthetic construct
  misc_feature 1..54: note = Synthetic: 61C9_p614-bla_R1
  source 1..54: mol_type = other DNA
cttttctacggggtctgacgcgtctcatgctcctcagtggaacgaaaactcacg

SEQ ID NO: 37 - DNA, 39 bp, synthetic construct
  misc_feature 1..39: note = Synthetic: 61D1_p614-bla_F1
  source 1..39: mol_type = other DNA
gtgaacactctcccggctgaaatctgctcgtcagtggtg

SEQ ID NO: 38 - DNA, 18 bp, synthetic construct
  misc_feature 1..18: note = Synthetic: 61D2_p614-bsmBI_R1
  source 1..18: mol_type = other DNA
gacgcgtctcatgctcct

SEQ ID NO: 39 - DNA, 18 bp, synthetic construct
  misc_feature 1..18: note = Synthetic: 61D2_p614-bsmBI_F1
  source 1..18: mol_type = other DNA
ctcccggctgaaatctgc

SEQ ID NO: 40 - DNA, 21 bp, synthetic construct
  misc_feature 1..21: note = Synthetic: 62B5_pBTK107_seq_F
  source 1..21: mol_type = other DNA
tggataaccgtagtcggtctc

SEQ ID NO: 41 - DNA, 23 bp, synthetic construct
  misc_feature 1..23: note = Synthetic: 62B6_pBTK107_seq_R
  source 1..23: mol_type = other DNA
ggatttgttcagaacgctcggtt

(19) United States
(12) Patent Application Publication
(10) Pub. No.: US 2026/0002166 A1
(43) Pub. Date: Jan. 1, 2026

(54) GENETIC ENGINEERING OF MARINE BACTERIA FOR BIOMATERIAL PRODUCTION, PROBIOTIC USE IN AQUACULTURE AND MARINE ENVIRONMENTAL RESTORATION

(71) Applicant: San Diego State University (SDSU) Foundation, dba San Diego State University Research Foundation, San Diego, CA (US)

(72) Inventors: Nicholas SHIKUMA, San Diego, CA (US); Amanda ALKER, San Diego, CA (US)

(21) Appl. No.: 18/844,517

(22) PCT Filed: Mar. 24, 2023
(86) PCT No.: PCT/US2023/016287

§ 371 Date: Sep. 6, 2024

Related U.S. Application Data
(60) Provisional application No. 63/323,653, filed on Mar. 25, 2022.
(60) Provisional application No. 63/438,170, filed on Jan. 10, 2023.

(51) Int. Cl. C12N 15/74; A01K 61/00; C12N 1/20; C12P 17/10; C12R 1/63

(52) U.S. Cl. CPC C12N 15/74; A01K 61/00; C12N 1/20; C12P 17/10; C12N 2800/90; C12R 2001/63

(57) ABSTRACT

In alternative embodiments, provided are compositions, including products of manufacture and kits, and methods, for producing enhanced marine bacterial strains with the ability to produce stimulatory products that amplify their probiotic effects for reef restoration and biotechnology applications. In alternative embodiments, genetically engineered marine bacteria (such as Nereida, Vibrio, Pseudoalteromonas and/or Roseobacter bacterium) as provided herein, and compositions, products of manufacture, kits and methods as provided herein, are used in biomaterial production, probiotic use in aquaculture and/or for environmental restoration purposes.

### RELATED APPLICATIONS

This is a national phase application claiming benefit of priority under 35 U.S.C. § 371 to Patent Convention Treaty (PCT) International Application Serial No: PCT/US23/16287, Mar. 24, 2023, which claims the benefit of priority to U.S. Provisional Application Ser. No. 63/323,653, Mar. 25, 2022; and U.S. Ser. No. 63/438,170, Jan. 10, 2023. The aforementioned applications are expressly incorporated herein by reference in its entirety and for all purposes.

### STATEMENT AS TO FEDERALLY SPONSORED RESEARCH

This invention was made with government support under 1942251 and 2017232404 awarded by the National Science Foundation (NSF); under N00014-20-1-2120 awarded by the Office of Naval Research; under R35GM146722 awarded by the National Institutes of Health. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 5, 2024, is named “5810.148444.xml” and is 96,943 bytes in size. The sequence listing contained in this .XML file is part of the specification and is hereby incorporated by reference herein in its entirety.

### TECHNICAL FIELD

This invention generally relates to marine ecology and microbiology. In alternative embodiments, provided are compositions, including products of manufacture and kits, and methods, for producing enhanced marine bacterial strains with the ability to produce stimulatory products that amplify their probiotic effects for reef restoration and biotechnology applications. In alternative embodiments, genetically engineered marine bacteria (such as Nereida, Vibrio, Pseudoalteromonas and/or Roseobacter bacterium) as provided herein, and compositions, products of manufacture, kits and methods as provided herein, are used in biomaterial production, probiotic use in aquaculture and/or for environmental restoration purposes.

### BACKGROUND

Climate change and human impacts have resulted in a rate of coral reef decline that has outpaced mitigation efforts, which has recently culminated in a call for new interventions to save coral reefs. An important factor dictating coral fitness is the quality of microbial symbionts that reside on and within corals, which has led coral restoration biologists to employ techniques that manipulate the coral microbiome such as treatments with probiotic bacteria. A desirable quality of microbes that are currently being considered as coral probiotics are those that generate cues that stimulate larval settlement and metamorphosis.

One of the best characterized molecules produced by bacteria that induces metamorphosis in a range of coral species is tetrabromopyrrole (TBP). While previous studies have shown that TBP purified from marine bacteria stimulates coral metamorphosis, the genetic tools have not been available in TBP-producing bacteria to link the TBP biosynthesis genes directly with the stimulation of coral metamorphosis.

The health of coral reef ecosystems worldwide has been declining drastically for the past few decades, leading to a crescendo of warnings from scientists about the fate of coral reefs as we know them. Since the 1950s, global coral reef coverage and the services provided by coral reef ecosystems have declined by half [1]. Frequent coral bleaching events have been followed by reports of drastically fewer coral larvae recruits [2], further dampening the likelihood of natural recovery for coral reef ecosystems.

An important facet of coral reef health is the coral holobiont, defined as the community of microorganisms, including dinoflagellates, prokaryotes, viruses and fungi that live on and within the corals [3]. These associations are crucial for a number of evolutionary, developmental and ecological interactions (reviewed in [4]). The consortia of microbes within the coral holobiont has been extensively studied [5]. The coral probiotic hypothesis suggests that as environmental conditions change, so too do the surrounding microbial communities. This dynamic relationship enables corals to select for the most advantageous microbial community resulting in a postulated short term adaptive advantage for the corals to resist against threats like disease [6].

A dire outlook on the progression and rate of coral decline has led scientists to realize the limitations of passive conservation strategies, such as the regional implementation of no-take zones [7, 8]. Integrated approaches combining passive strategies with active and riskier interventions such as the manipulation of the coral microbiome have been proposed to help offset further coral reef decline [7, 9]. Probiotic treatment with microbial consortia have been proposed as a novel solution towards coral decline, whereby Beneficial Microorganisms for Corals (BMCs) are isolated and screened for potential probiotic mechanisms [10-12]. Currently, probiotic bacterial strains are already being tested [13, 14], and in some cases implemented [15] to mitigate the decline of coral health. However, we currently lack the mechanistic data that supports the usage of probiotic bacteria for promoting coral health. One of the desirable characteristics among BMCs is their ability to produce products that promote the settlement of larvae and metamorphosis from larval to juvenile stages, which could be utilized in both environmental [16] and aquaculture settings [17, 18].

The brominated marine pyrroles/phenols (bmp) gene cluster was identified and characterized previously [26] and the bmp1-4 genes were determined to be important for TBP biosynthesis in Pseudoalteromonas sp. PS5 [27]. The gene bmp2 is a flavin-dependent halogenase capable of tetrabromination, via a novel decarboxylative bromination reaction demonstrated through total in vitro reconstitution [27]. While Pseudoalteromonas sp. PS5 biofilms were shown to influence metamorphosis [23], it has not been shown whether the TBP biosynthesis genes are responsible for stimulating coral metamorphosis. The functional link between the bmp biosynthesis genes and coral metamorphosis has not yet been explicitly tested because (1) genetic manipulation techniques in a metamorphosis-inducing bacterium with the TBP biosynthesis genes has not been developed and (2) our ability to capture and rear coral larvae has become comprehensive and predictable only in the last decade (Marhaver).

Some bacteria forming biofilms composed of a single species can induce coral metamorphosis [19-21]. Bacterial species from the genus Pseudoalteromonas have been shown to stimulate coral metamorphosis and appear to be promising candidates for probiotic use [20, 22-24]. Pseudoalteromonas sp. PS5 [23] and related strains [22] are capable of producing a compound called tetrabromopyrrole (TBP), which has been associated with coral metamorphosis. Biofilms, fractionated extracts and synthesized TBP all induce robust metamorphosis in both Pacific and Atlantic corals.

Although TBP induces both attached and unattached metamorphosis phenotypes and is the subject of debate as an ecologically relevant signal [25], TBP has still remained a top consideration as a potential signal for coral development.

Species within and related to the Nereida genus have been found in association with diverse marine eukaryotes. Nereida ignava CECT 5292 is related to an uncultured gall symbiont from red algae (6). Related species from the genus Octodecabacter were isolated from an ascidian or compose 70-80% of the microbiome of a brittle star (11, 12). Roseobacter species are associated with algae and reef building corals where they are thought to play important roles global sulfur cycling, in part through degradation of dimethylsulfoniopropionate (DMSP) (13).

### SUMMARY

In alternative embodiments, transposon vectors comprising, or consisting of:
(a) a nucleic acid sequence comprising or consisting of a sequence as set forth in SEQ ID NO:1; SEQ ID NO:2; SEQ ID NO:3; SEQ ID NO:4; SEQ ID NO:5; SEQ ID NO:6; SEQ ID NO:7; SEQ ID NO: 8; SEQ ID NO: 9; SEQ ID NO:10; SEQ ID NO:11 or SEQ ID NO:12,
(b) a plurality of modular elements operatively linked to each other, wherein the plurality of modular elements comprise:
   a Type-2 broad host range CP25 promoter,
   a Type-3 GFP or mRuby protein coding sequence (CDS) or structural gene (or a protein coding sequence),
   a Type-4 terminator,
   a Type-1 and a Type-5 connector, and
   a Type-8 RSF1010 backbone,
wherein optionally the transposon vector comprises or has contained therein a structural gene or protein coding sequence, wherein optionally the structural gene or protein coding sequence is selected from the group consisting of: a gene encoding or protein coding sequence for tetrabromopyrrole (TBP), bmp (2,2-Bis(bromomethyl)-1,3-propanediol) genes, metamorphosis associated contractile structures gene B, metamorphosis associated contractile structures gene S, metamorphosis associated contractile structures gene R, LPS (lipopolysaccharide) genes, EPS (extracellular polymeric substances) genes, OMV (outer membrane vesicle) genes, omp (outer membrane protein) genes, RNA polymerase sigma factor rpoS, RNA polymerase sigma factor rpoE, CRISPR Cas9 variants, CRISPR variants, and a tag,
wherein optionally the tag is or comprises: FLAG, histidine or polyhistidine (His), Sumo, GST (glutathione S transferase), SNAP-tag™ or CLICK-tag™.

In alternative embodiments, of transposon vectors as provided herein:
the vector comprises a modular assembly comprising:
(a) 5′-Type1 (linker)-Type2 (Bmp1p, macBp, or CP25p)-Type3 (gfp, mRuby, or nanoluc)-Type4 (T7-terminator)-Type5 (linker)-Type6-7-Type8 (Tn7/Tn10/RSF1010)-3′;
(b) a Type2-CP25p or Type8-Tn7 vector comprising:
5′-Type1 (linker)-Type2 (CP25p)-Type3 (gfp)-Type4 (T7-terminator)-Type5 (linker)-Type6-7-Type8 (Tn7)-3′,
5′-Type1 (linker)-Type2 (CP25p)-Type3 (mRuby)-Type4 (T7-terminator)-Type5 (linker)-Type6-7-Type8 (Tn7)-3′, or
5′-Type1 (linker)-Type2 (CP25p)-Type3 (nanoluc)-Type4 (T7-terminator)-Type5 (linker)-Type6-7-Type8 (Tn7)-3′;
(b) a Type2-CP25p or Type8-Tn10 vector comprising:
5′-Type1 (linker)-Type2 (CP25p)-Type3 (gfp)-Type4 (T7-terminator)-Type5 (linker)-Type6-7-Type8 (Tn10)-3′,
5′-Type1 (linker)-Type2 (CP25p)-Type3 (mRuby)-Type4 (T7-terminator)-Type5 (linker)-Type6-7-Type8 (Tn10)-3′, or
5′-Type1 (linker)-Type2 (CP25p)-Type3 (nanoluc)-Type4 (T7-terminator)-Type5 (linker)-Type6-7-Type8 (Tn10)-3′;
(c) a Type2-CP25p or a Type8-RSF1010 vector comprising:
5′-Type1 (linker)-Type2 (CP25p)-Type3 (gfp)-Type4 (T7-terminator)-Type5 (linker)-Type6-7-Type8 (RSF1010)-3′,
5′-Type1 (linker)-Type2 (CP25p)-Type3 (mRuby)-Type4 (T7-terminator)-Type5 (linker)-Type6-7-Type8 (RSF1010)-3′, or
5′-Type1 (linker)-Type2 (CP25p)-Type3 (nanoluc)-Type4 (T7-terminator)-Type5 (linker)-Type6-7-Type8 (RSF1010)-3′;
(d) a Type2-Bmp1p or a Type8-Tn7 vector comprising:
5′-Type1 (linker)-Type2 (Bmp1p)-Type3 (gfp)-Type4 (T7-terminator)-Type5 (linker)-Type6-7-Type8 (Tn7)-3′,
5′-Type1 (linker)-Type2 (Bmp1p)-Type3 (mRuby)-Type4 (T7-terminator)-Type5 (linker)-Type6-7-Type8 (Tn7)-3′, or
5′-Type1 (linker)-Type2 (Bmp1p)-Type3 (nanoluc)-Type4 (T7-terminator)-Type5 (linker)-Type6-7-Type8 (Tn7)-3′;
(e) a Type2-Bmp1p or Type8 Tn10 vector comprising:
5′-Type1 (linker)-Type2 (Bmp1p)-Type3 (gfp)-Type4 (T7-terminator)-Type5 (linker)-Type6-7-Type8 (Tn10)-3′,
5′-Type1 (linker)-Type2 (Bmp1p)-Type3 (mRuby)-Type4 (T7-terminator)-Type5 (linker)-Type6-7-Type8 (Tn10)-3′, or
5′-Type1 (linker)-Type2 (Bmp1p)-Type3 (nanoluc)-Type4 (T7-terminator)-Type5 (linker)-Type6-7-Type8 (Tn10)-3′;
(f) a Type2-Bmp1p or a Type8-RSF1010 vector comprising:
5′-Type1 (linker)-Type2 (Bmp1p)-Type3 (gfp)-Type4 (T7-terminator)-Type5 (linker)-Type6-7-Type8 (RSF1010)-3′,
5′-Type1 (linker)-Type2 (Bmp1p)-Type3 (mRuby)-Type4 (T7-terminator)-Type5 (linker)-Type6-7-Type8 (RSF1010)-3′, or
5′-Type1 (linker)-Type2 (Bmp1p)-Type3 (nanoluc)-Type4 (T7-terminator)-Type5 (linker)-Type6-7-Type8 (RSF1010)-3′
(g) a Type2-MacBp or a Type8-Tn7 vector comprising:
5′-Type1 (linker)-Type2 (MacBp)-Type3 (gfp)-Type4 (T7-terminator)-Type5 (linker)-Type6-7-Type8 (Tn7)-3′,
5′-Type1 (linker)-Type2 (MacBp)-Type3 (mRuby)-Type4 (T7-terminator)-Type5 (linker)-Type6-7-Type8 (Tn7)-3′, or
5′-Type1 (linker)-Type2 (MacBp)-Type3 (nanoluc)-Type4 (T7-terminator)-Type5 (linker)-Type6-7-Type8 (Tn7)-3′;
(h) a Type2-MacBp or a Type8 Tn10 vector comprising:
5′-Type1 (linker)-Type2 (MacBp)-Type3 (gfp)-Type4 (T7-terminator)-Type5 (linker)-Type6-7-Type8 (Tn10)-3′,
5′-Type1 (linker)-Type2 (MacBp)-Type3 (mRuby)-Type4 (T7-terminator)-Type5 (linker)-Type6-7-Type8 (Tn10)-3′, or
5′-Type1 (linker)-Type2 (MacBp)-Type3 (nanoluc)-Type4 (T7-terminator)-Type5 (linker)-Type6-7-Type8 (Tn10)-3′;
(i) a Type2-MacBp or Type8-RSF1010 vector comprising:
5′-Type1 (linker)-Type2 (MacBp)-Type3 (gfp)-Type4 (T7-terminator)-Type5 (linker)-Type6-7-Type8 (RSF1010)-3′,
5′-Type1 (linker)-Type2 (MacBp)-Type3 (mRuby)-Type4 (T7-terminator)-Type5 (linker)-Type6-7-Type8 (RSF1010)-3′, or
5′-Type1 (linker)-Type2 (MacBp)-Type3 (nanoluc)-Type4 (T7-terminator)-Type5 (linker)-Type6-7-Type8 (RSF1010)-3′.

In alternative embodiments, of transposon vectors as provided herein, the vector comprises a modular assembly and a sequence selected from the group consisting of: a Type-1 linker vector (SEQ ID NO:3); a Type-2 CP25 promoter vector (SEQ ID NO:4); a Type-2 macB promoter vector (SEQ ID NO:5; Type-2 bmp1 promoter vector (SEQ ID NO:6); Type-3 coding GFP vector (SEQ ID NO:7); Type-3 coding nanoluc vector (SEQ ID NO:8); Type-3 coding mRuby vector (SEQ ID NO:9); Type-4 terminator vector (SEQ ID NO:10); Type-5 linker vector (SEQ ID NO: 11) and Type-8 RSF1010 backbone vector (SEQ ID NO:12).

In alternative embodiments, provided are cells comprising or having contained therein a transposon vector comprising, or consisting of, a nucleic acid sequence as set forth in SEQ ID NO:1 or SEQ ID NO:2, or a transposon vector as provided or described herein; and optionally the cell is a bacterial cell, optionally a marine bacterium, optionally a marine bacterium of the genus or family Nereida, Roseobacter, Pseudoalteromonas and/or Vibrio, optionally Pseudoalteromonas sp. PS5.

In alternative embodiments, provided are methods for genetically modifying a cell comprising inserting into the cell a transposon vector as provided herein, wherein optionally the cell is a bacterial cell, optionally a marine bacterium, optionally a bacterium of the genus or family Nereida, Roseobacter, Pseudoalteromonas and/or Vibrio, optionally Pseudoalteromonas sp. PS5.

In alternative embodiments, provided are kits or products of manufacture comprising a transposon vector as described or provided herein.

In alternative embodiments, provided are methods for enhancing growth of a coral or enhancing growth or a coral reef, or enhancing or stimulating coral metamorphosis, comprising exposing the coral or coral reef to a genetically engineered marine bacterial cell (bacterium) that expresses and/or secretes into the extracellular milieu higher than wild type levels of tetrabromopyrrole (TBP),
and optionally the marine bacterium is of the genus or family Nereida, Roseobacter, Pseudoalteromonas and/or Vibrio, optionally Pseudoalteromonas sp. PS5,
and optionally the marine bacterium comprises or has contained therein a transposon vector as described or provided herein.

In alternative embodiments, provided are transposon vectors as described or provided herein, cells as described or provided herein, or kits or products of manufacture as described or provided herein, for use in enhancing growth of a coral or enhancing growth of a coral reef, or enhancing or stimulating coral metamorphosis.

In alternative embodiments, provided are uses of a transposon vector as described or provided herein, a cell as described or provided herein, or a kit or product of manufacture as described or provided herein, for enhancing growth of a coral or enhancing growth of a coral reef, or enhancing or stimulating coral metamorphosis.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A illustrates a scanning electron micrograph image of Nereida sp. MMG025;

FIG. 1B illustrates a maximum likelihood phylogeny constructed using the Codon tree method though the Pathosystems Resource Integration Center (PATRIC) (NIAID, NIH) using 100 single-copy genes and proteins identified by PGFams (Global Protein Families, in the PATRIC database),
as described in detail in Example 1, below.

FIG. 2A-C illustrate that Pseudoalteromonas sp. PS5 strain lacking the brominase gene bmp2 is unable to produce TBP and does not induce coral metamorphosis:

FIG. 2A schematically illustrates a model of the TBP biosynthesis gene cluster in Pseudoalteromonas sp. PS5 including bmp1-4 and bmp8-10 genes;

FIG. 2B graphically illustrates data showing that Pseudoalteromonas sp. PS5 produces 30-fold more TBP than P. luteoviolacea strain HI1 and mutation of the bmp2 gene ablates TBP production in both strains; error bars represent standard deviation of the mean in plots FIG. 2B and FIG. 2C; and

FIG. 2C graphically illustrates data showing metamorphosis (%) of Porites astreoides larvae in response to Pseudoalteromonas sp. PS5 wild type and Δbmp2 strains; MARINE BROTH 2216™ (Difco) growth media or Filtered Sea Water (FSW) alone are included as controls,
as described in detail in Example 2, below.

FIG. 3 a schematic overview of the exemplary modular MMK system and integration into diverse bacteria for experiment testing, as discussed in detail in Example 4, below.

FIG. 4 illustrate conjugation and fluorescent protein expression in numerous Pseudoalteromonas, Roseobacter and Vibrio species, as discussed in detail in Example 4, below.

FIG. 5A schematically illustrates the whole genome phylogeny of 10 strains selected for manipulation and successfully transformed using exemplary methods as provided herein; all strains are known for their interaction with a range of marine biota, for example, Gammaproteobacteria strains are highlighted in purple and Alphaproteobacteria strains are shown in orange; scale bar is 0.3 and bootstraps were generated using the rapid-bootstrapping method; and,

FIG. 5B illustrates images of Pseudoalteromonas, Roseobacter and Vibrio species cells having stage-1 plasmids that are stably replicated and express fluorescent proteins, as discussed in detail in Example 4, below.

FIG. 6A-B illustrate CRISPRi reduces gene expression in Pseudoalteromonas:

FIG. 6A illustrates an exemplary agar plate of P. luteoviolacea comparing the control (sgRNA-GFP) to the violacein knockdown (sgRNA-VioA5);

FIG. 6B graphically illustrates quantification of violacein extracted from overnight cultures of P. luteoviolacea containing a gfp control sgRNA plasmid versus an sgRNA targeting VioA,
as discussed in detail in Example 4, below.

FIG. 7A-C illustrate that two Roseobacter species that have not previously been shown to stimulate animal metamorphosis, Leisingera sp. 204H and P. gallaeciensis ATCC 700781, were able to stimulate the metamorphosis of Hydroides larvae:

FIG. 7A graphically illustrates data showing % Hydroides metamorphosis stimulated by several different species, as found using an OD1 Hydroides biofilm assay;

FIG. 7B schematically illustrates the exemplary OD1 Hydroides biofilm assay;

FIG. 7C illustrate images showing that after incubation of bacteria and larvae for 24 hours,
as discussed in detail in Example 4, below.

FIG. 8A-E illustrates a schematic overview of the modular plasmid system and quantitative promoter measurements:

FIG. 8A schematically illustrates modular GGA plasmid parts with flanking BsaI cut sites (dashed lines);

FIG. 8B schematically illustrates GOLDEN GATE ASSEMBLY™ (Thermo Fisher Scientific, Waltham, MA) is performed in a one-tube reaction by digesting the backbone and insert part plasmids with BsaI and ligating with T4 ligase;

FIG. 8C schematically illustrates that a modular stage 1 plasmid is complete when all overlapping inserts are successfully assembled in order;

FIG. 8D graphically illustrates data from a biofilm luciferase assay of P. luteoviolacea strains expressing replicative plasmids with different constitutive promoters driving a Nluc gene (CP25-Nluc-T7, PA3-Nluc-T7, Ptac-Nluc-T7),
as discussed in detail in Example 5, below.

FIG. 9A-D illustrate data showing: CRISPRi knockdown of secondary metabolite production in P. luteoviolacea:

FIG. 9A schematically illustrates how modular CRISPRi parts were adapted from a previous study to include dCas9-bla and Ptac-sgRNA parts, pMMK601 and pMMK602, respectively; part plasmids are combined and a GOLDEN GATE ASSEMBLY™ (Thermo Fisher Scientific, Waltham, MA) is performed with BsmBI;

FIG. 9B schematically illustrates how the CRISPRi system was assembled with an sgRNA targeting the vioA gene (pMMK603) and employed to knock down violacein production in P. luteoviolacea;

FIG. 9C illustrates an image of an agar plate showing knockdown of vioA resulted in a visible difference in purple pigment produced in bacteria grown on agar plates; and

FIG. 9D graphically illustrates data from extraction of overnight cultures revealed a significant reduction in violacein production (measured at 580 nm) between wildtype containing a non-targeting gfp plasmid and knockdown,
as discussed in detail in Example 5, below.

FIG. 10A-D illustrate data showing functional knockdown of MACs and visualization of P. luteoviolacea during the tubeworm-microbe interaction:

FIG. 10A schematically illustrates P. luteoviolacea and the production of MACs, which induce tubeworm metamorphosis;

FIG. 10B graphically illustrates data from a single strain biofilm metamorphosis assay with CRISPRi modular plasmid targeting gene, macB induced significantly less metamorphosis in H. elegans; and

FIG. 10C-D illustrate fluorescence micrographs of juvenile Hydroides elegans imaged 24 hours after the competent larvae were exposed to inductive biofilms of P. luteoviolacea containing constitutively expressed (FIG. 10C) CP25-gfp (FIG. 10D) CP25-Nluc; arrows show accumulation of fluorescent bacteria in the intestinal tract,
as discussed in detail in Example 5, below.

FIG. 11A-B illustrate data showing that diverse marine Proteobacteria are amenable to plasmid uptake and stable replication of toolkit plasmids:

FIG. 11A illustrates maximum likelihood whole genome phylogeny of 12 strains selected for manipulation and successfully transformed in this study; and

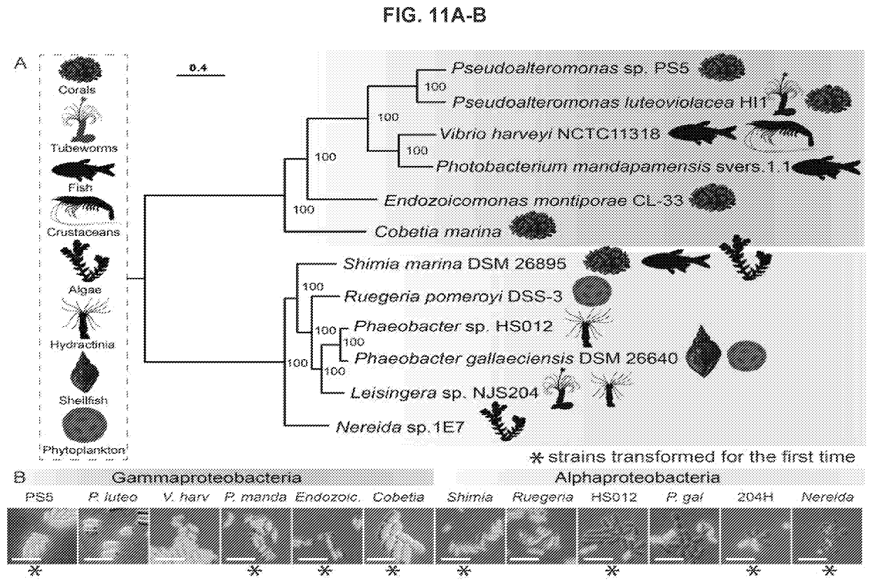

FIG. 11B illustrates fluorescence microscopy images of overnight cultures containing constitutively expressed RSF1010 on fluorescence vector,
as discussed in detail in Example 5, below.

Like reference symbols in the various drawings indicate like elements.

### DETAILED DESCRIPTION

In alternative embodiments, provided are compositions, including products of manufacture and kits, and methods, for the genetic modification, or genetic engineering of, marine bacteria. In alternative embodiments, the genetically engineered marine bacteria (such as Nereida, Vibrio, Pseudoalteromonas and/or Roseobacter bacterium), the compositions, products of manufacture, kits and methods as provided herein are used in biomaterial production, probiotic use in aquaculture and/or for environmental restoration purposes. The ability to genetically manipulate probiotic marine bacteria, as provided by products of manufacture and kits, and methods, as provided herein opens the door to the production of enhanced strains with the ability to produce stimulatory products that amplify their probiotic effects for reef restoration and biotechnology applications.

In alternative embodiments, provided is a molecular toolkit for genetic modification of marine bacteria such as Nereida bacterium, and molecular toolkit as provided herein can be used as a standardized molecular cloning platform (designated herein “Marine Modification Toolkit” or MMK) for modifying marine bacteria.

Using the MMKs as provided herein it is now possible to genetically modify diverse marine bacteria from the ocean, including bacteria that have never been modified before.

In alternative embodiments, we combine the usage of genetic tools (suicide plasmids, conjugative strains of E. coli) that have been used with commonly modified bacteria like Pseudomonas aeruginosa with the uncommon and difficult to grow bacterium Pseudoalteromonas sp. PS5. In alternative embodiments, provided are optimized techniques for conjugation and usage of specific genetic tools (modular plasmids) that have allowed us to rapidly test the efficacy of genetic modification. We combined specific genetic plasmids and elements with specific conjugative strains (E. coli MFDpir) to enhance the genetic modification procedure.

In alternative embodiments, provided are new modular plasmids (termed “Tn7” and “Tn10”) that can help with the genetic modification process. This modular plasmid system is able to genetically modify a range of marine bacteria from diverse taxa including Vibrio, Roseobacter and Pseudoalteromonas bacteria.

Provided herein are data showing proof of concept genetic manipulation in multiple species of marine bacteria, including Vibrio, Pseudoalteromonas and Roseobacter species. We demonstrate the functionality of MMKs as provided herein as applied to marine bacteria that perform important symbiotic functions with marine plants or animals. Specifically, we demonstrate that a number of previously tractable and intractable Roseobacter, Pseudoalteromonas and Vibrio species (particularly, Pseudoalteromonas sp. PS5, which before this invention was intractable to genetic recombinant manipulation) can be genetically manipulated using MMK and methods as provided herein to stably carry a broad host range plasmids and to express heterologous nucleic acids (or genes), or specifically, to express fluorescent proteins and nanoluciferase genes.

Also described herein are new transposon vectors (designated Tn7 and Tn10) that are compatible with the standardized genetic parts system and can stably integrate into the genome of a marine bacterium such as a Pseudoalteromonas and/or a Roseobacter species.

### Exemplary Modular Assemblies for Novel Plasmids as Provided Herein

Below are the combinations of modular components of novel plasmids as provided herein:
5′-Type1 (linker)-Type2 (Bmp1p, macBp, or CP25p)-Type3 (gfp, mRuby, or nanoluc)-Type4 (T7-terminator)-Type5 (linker)-Type6-7-Type8 (Tn7/Tn10/RSF1010)-3′

### Type2-CP25p

### Type8-Tn7

5′-Type1 (linker)-Type2 (CP25p)-Type3 (gfp)-Type4 (T7-terminator)-Type5 (linker)-Type6-7-Type8 (Tn7)-3′
5′-Type1 (linker)-Type2 (CP25p)-Type3 (mRuby)-Type4 (T7-terminator)-Type5 (linker)-Type6-7-Type8 (Tn7)-3′
5′-Type1 (linker)-Type2 (CP25p)-Type3 (nanoluc)-Type4 (T7-terminator)-Type5 (linker)-Type6-7-Type8 (Tn7)-3′

### Type2-CP25p

### Type8-Tn10

5′-Type1 (linker)-Type2 (CP25p)-Type3 (gfp)-Type4 (T7-terminator)-Type5 (linker)-Type6-7-Type8 (Tn10)-3′
5′-Type1 (linker)-Type2 (CP25p)-Type3 (mRuby)-Type4 (T7-terminator)-Type5 (linker)-Type6-7-Type8 (Tn10)-3′
5′-Type1 (linker)-Type2 (CP25p)-Type3 (nanoluc)-Type4 (T7-terminator)-Type5 (linker)-Type6-7-Type8 (Tn10)-3′

### Type2-CP25p

### Type8-RSF1010

5′-Type1 (linker)-Type2 (CP25p)-Type3 (gfp)-Type4 (T7-terminator)-Type5 (linker)-Type6-7-Type8 (RSF1010)-3′
5′-Type1 (linker)-Type2 (CP25p)-Type3 (mRuby)-Type4 (T7-terminator)-Type5 (linker)-Type6-7-Type8 (RSF1010)-3′
5′-Type1 (linker)-Type2 (CP25p)-Type3 (nanoluc)-Type4 (T7-terminator)-Type5 (linker)-Type6-7-Type8 (RSF1010)-3′

### Type2-Bmp1p

### Type8-Tn7

5′-Type1 (linker)-Type2 (Bmp1p)-Type3 (gfp)-Type4 (T7-terminator)-Type5 (linker)-Type6-7-Type8 (Tn7)-3′
5′-Type1 (linker)-Type2 (Bmp1p)-Type3 (mRuby)-Type4 (T7-terminator)-Type5 (linker)-Type6-7-Type8 (Tn7)-3′
5′-Type1 (linker)-Type2 (Bmp1p)-Type3 (nanoluc)-Type4 (T7-terminator)-Type5 (linker)-Type6-7-Type8 (Tn7)-3′

### Type2-Bmp1p

### Type8 Tn10

5′-Type1 (linker)-Type2 (Bmp1p)-Type3 (gfp)-Type4 (T7-terminator)-Type5 (linker)-Type6-7-Type8 (Tn10)-3′
5′-Type1 (linker)-Type2 (Bmp1p)-Type3 (mRuby)-Type4 (T7-terminator)-Type5 (linker)-Type6-7-Type8 (Tn10)-3′
5′-Type1 (linker)-Type2 (Bmp1p)-Type3 (nanoluc)-Type4 (T7-terminator)-Type5 (linker)-Type6-7-Type8 (Tn10)-3′

### Type2-Bmp1p

### Type8-RSF1010

5′-Type1 (linker)-Type2 (Bmp1p)-Type3 (gfp)-Type4 (T7-terminator)-Type5 (linker)-Type6-7-Type8 (RSF1010)-3′
5′-Type1 (linker)-Type2 (Bmp1p)-Type3 (mRuby)-Type4 (T7-terminator)-Type5 (linker)-Type6-7-Type8 (RSF1010)-3′
5′-Type1 (linker)-Type2 (Bmp1p)-Type3 (nanoluc)-Type4 (T7-terminator)-Type5 (linker)-Type6-7-Type8 (RSF1010)-3′

### Type2-MacBp

### Type8-Tn7

5′-Type1 (linker)-Type2 (MacBp)-Type3 (gfp)-Type4 (T7-terminator)-Type5 (linker)-Type6-7-Type8 (Tn7)-3′
5′-Type1 (linker)-Type2 (MacBp)-Type3 (mRuby)-Type4 (T7-terminator)-Type5 (linker)-Type6-7-Type8 (Tn7)-3′
5′-Type1 (linker)-Type2 (MacBp)-Type3 (nanoluc)-Type4 (T7-terminator)-Type5 (linker)-Type6-7-Type8 (Tn7)-3′

### Type2-MacBp

### Type8-Tn10

5′-Type1 (linker)-Type2 (MacBp)-Type3 (gfp)-Type4 (T7-terminator)-Type5 (linker)-Type6-7-Type8 (Tn10)-3′
5′-Type1 (linker)-Type2 (MacBp)-Type3 (mRuby)-Type4 (T7-terminator)-Type5 (linker)-Type6-7-Type8 (Tn10)-3′
5′-Type1 (linker)-Type2 (MacBp)-Type3 (nanoluc)-Type4 (T7-terminator)-Type5 (linker)-Type6-7-Type8 (Tn10)-3′

### Type2-MacBp

### Type8-RSF1010

5′-Type1 (linker)-Type2 (MacBp)-Type3 (gfp)-Type4 (T7-terminator)-Type5 (linker)-Type6-7-Type8 (RSF1010)-3′
5′-Type1 (linker)-Type2 (MacBp)-Type3 (mRuby)-Type4 (T7-terminator)-Type5 (linker)-Type6-7-Type8 (RSF1010)-3′
5′-Type1 (linker)-Type2 (MacBp)-Type3 (nanoluc)-Type4 (T7-terminator)-Type5 (linker)-Type6-7-Type8 (RSF1010)-3′

### Below are Exemplary Novel Plasmids as Provided Herein:

The sequence of the exemplary Type-1 linker vector is below. The linker sequence enclosed by BsaI cut sites for modular assembly is indicated by an underline. BsaI restriction site recognition sites are indicated in bold:

(SEQ ID NO: 3)
TCGGTCTCACCCTGAATTCGCATCTAGACTGATGAGACGTGGTAGAGCCA
CAAACAGCCGGTACAAGCAACGATCTCCAGGACCATCTGAATCATGCGCG
GATGACACGAACTCACGACGGCGATCACAGACATTAACCCACAGTACAG
ACACTGCGACAACGTGGCAATTCGTCGCAATACAACGTGAGACCAGACC
AATAAAAAACGCCCGGCGGCAACCGAGCGTTCTGAACAAATCCAGATGG
AGTTCTGAGGTCATTACTGGATCTATCAACAGGAGTCCAAGCGAGCTCGA
TATCAAATTACGCCCCGCCCTGCCACTCATCGCAGTACTGTTGTAATTCAT
TAAGCATTCTGCCGACATGGAAGCCATCACAAACGGCATGATGAACCTGA
ATCGCCAGCGGCATCAGCACCTTGTCGCCTTGCGTATAATATTTGCCCATG
GTGAAAACGGGGGCGAAGAAGTIGTCCATATTGGCCACGTTTAAATCAAA
ACTGGTGAAACTCACCCAGGGATTGGCTGAAACGAAAAACATATTCTCAA
TAAACCCTTTAGGGAAATAGGCCAGGTTTTCACCGTAACACGCCACATCTT
GCGAATATATGTGTAGAAACTGCCGGAAATCGTCGTGGTATTCACTCCAG
AGCGATGAAAACGTTTCAGTTTGCTCATGGAAAACGGTGTAACAAGGGTG
AACACTATCCCATATCACCAGCTCACCGTCTTTCATTGCCATACGAAATTC
CGGATGAGCATTCATCAGGCGGGCAAGAATGTGAATAAAGGCCGGATAA
AACTTGTGCTTATTTTTCTTTACGGTCTTTAAAAAGGCCGTAATATCCAGC
TGAACGGTCTGGTTATAGGTACATTGAGCAACTGACTGAAATGCCTCAAA
ATGTTCTTTACGATGCCATTGGGATATATCAACGGTGGTATATCCAGTGAT
TTTTTTCTCCATTTTAGCTTCCTTAGCTCCTGAAAATCTCGATAACTCAAA
AAATACGCCCGGTAGTGATCTTATTTCATTATGGTGAAAGTTGGAACCTCT
TACGTGCCCGATCAATCATGACCAAAATCCCTTAACGTGAGTTTTCGTTCC
ACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTC
TGCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGC
TGAACGGGGGGTTCGTGCACACAGCCCAGCTTGGAGCGAACGACCTACAC
CGAACTGAGATACCTACAGCGTGAGCTATGAGAAAGCGCCACGCTTCCCG
AAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGG
TTCCTGGCCTTTTGCTGGCCTTTTGCTCACATGTTCTTTCCTGCGTTATCC
CCTGATTCTGTGGATAACCGTAG

The sequence of the exemplary Type-2 CP25 promoter vector is below. The promoter sequence enclosed by BsaI cut sites for modular assembly is indicated by an underline. BsaI restriction site recognition sites are indicated in bold:

(SEQ ID NO: 4)
GTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGT
TCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACATG
TTCTTTCCTGCGTTATCCCCTGATTCTGTGGATAACCGTAGTCGGTCTCAA
ACGCTTTGGCAGTTTATTCTTGACATGTAGTGAGGGGGCTGGTATAATCAC
ATAGTACTGTTATACAGAAACAGAGGAGATATTACATATGTGAGACCAGAC
CAATAAAAAACGCCCGGCGGCAACCGAGCGTTCTGAACAAATCCAGATG
GAGTTCTGAGGTCATTACTGGATCTATCAACAGGAGTCCAAGCGAGCTCG
ATATCAAATTACGCCCCGCCCTGCCACTCATCGCAGTACTGTTGTAATTCA
TTAAGCATTCTGCCGACATGGAAGCCATCACAAACGGCATGATGAACCTG
AATCGCCAGCGGCATCAGCACCTTGTCGCCTTGCGTATAATATTTGCCCAT
GGTGAAAACGGGGGCGAAGAAGTIGTCCATATTGGCCACGTTTAAATCAA
AACTGGTGAAACTCACCCAGGGATTGGCTGAAACGAAAAACATATTCTCA
ATAAACCCTTTAGGGAAATAGGCCAGGTTTTCACCGTAACACGCCACATC
TTGCGAATATATGTGTAGAAACTGCCGGAAATCGTCGTGGTATTCACTCCA
GAGCGATGAAAACGTTTCAGTTTGCTCATGGAAAACGGTGTAACAAGGGT
GAACACTATCCCATATCACCAGCTCACCGTCTTTCATTGCCATACGAAATT
CCGGATGAGCATTCATCAGGCGGGCAAGAATGTGAATAAAGGCCGGATA
AAACTTGTGCTTATTTTTCTTTACGGTCTTTAAAAAGGCCGTAATATCCAG
CTGAACGGTCTGGTTATAGGTACATTGAGCAACTGACTGAAATGCCTCAA
AATGTTCTTTACGATGCCATTGGGATATATCAACGGTGGTATATCCAGTGA
TTTTTTTCTCCATTTTAGCTTCCTTAGCTCCTGAAAATCTCGATAACTCAA
AAAATACGCCCGGTAGTGATCTTATTTCATTATGGTGAAAGTTGGAACCTC
TTACGTGCCCGATCAATCATGACCAAAATCCCTTAAC

The sequence of the exemplary Type-2 macB promoter vector is below. The promoter sequence enclosed by BsaI cut sites for modular assembly is indicated by an underline. BsaI restriction site recognition sites are indicated in bold:

(SEQ ID NO: 5)
GTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGT
TCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACATG
TTCTTTCCTGCGTTATCCCCTGATTCTGTGGATAACCGTAGTCGGTCTCAA
ACGGAAGTTTCTGCGGTGCTTTTAAACTATGAGCCGAGAATTATCCTTGAG
GATATTTCGTTTGATATGTCAGATGTGTACGAAGGTGCCTTATTAATAGAG
CTCACTTATTTGATCCGCAAAACCAATAGCCGCAGCAATATGGTGTTTCCG
TTTTATCTCGCCGAGCAGTCTGTTTAAACCCGCGTTTCAAACATTTGGTAT
CACCCACAGAATCACCCATTTCGACCTTGCTCGCCAAGCAATATGCAGACC
CGCACCGCTTATCCTTTTACCAAGGTTAAATTGACCTAGTAGTGCATAGGT
ATGCTCATTAATAACATTAATAAGGTAATCTTATGTGAGACCAGACCAAT
AAAAAACGCCCGGCGGCAACCGAGCGTTCTGAACAAATCCAGATGGAGTT
CTGAGGTCATTACTGGATCTATCAACAGGAGTCCAAGCGAGCTCGATATC
AAATTACGCCCCGCCCTGCCACTCATCGCAGTACTGTTGTAATTCATTAAG
CATTCTGCCGACATGGAAGCCATCACAAACGGCATGATGAACCTGAATCG
CCAGCGGCATCAGCACCTTGTCGCCTTGCGTATAATATTTGCCCATGGTGA
AAACGGGGGCGAAGAAGTTGTCCATATTGGCCACGTTTAAATCAAAACTG
GTGAAACTCACCCAGGGATTGGCTGAAACGAAAAACATATTCTCAATAAA
CCCTTTAGGGAAATAGGCCAGGTTTTCACCGTAACACGCCACATCTTGCG
AATATATGTGTAGAAACTGCCGGAAATCGTCGTGGTATTCACTCCAGAGC
GATGAAAACGTTTCAGTTTGCTCATGGAAAACGGTGTAACAAGGGTGAAC
ACTATCCCATATCACCAGCTCACCGTCTTTCATTGCCATACGAAATTCCGG
ATGAGCATTCATCAGGCGGGCAAGAATGTGAATAAAGGCCGGATAAAAC
TTGTGCTTATTTTTCTTTACGGTCTTTAAAAAGGCCGTAATATCCAGCTGA
ACGGTCTGGTTATAGGTACATTGAGCAACTGACTGAAATGCCTCAAAATG
TTCTTTACGATGCCATTGGGATATATCAACGGTGGTATATCCAGTGATTTT
TTTCTCCATTTTAGCTTCCTTAGCTCCTGAAAATCTCGATAACTCAAAAAA
TACGCCCGGTAGTGATCTTATTTCATTATGGTGAAAGTTGGAACCTCTTAC
GTGCCCGATCAATCATGACCAAAATCCCTTAAC

The sequence of the exemplary Type-2 bmp1 promoter vector is below. The promoter sequence enclosed by BsaI cut sites for modular assembly is indicated by an underline. BsaI restriction site recognition sites are indicated in bold:

(SEQ ID NO: 6)
GTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAA
AAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGT
TCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGG
CGATAAGTCGTGTCTTACCGGGTTGGACTCAAGACGATAGTTACCGGATA
AGGCGCAGCGGTCGGGCTGAACGGGGGGTTCGTGCACACAGCCCAGCTTG
GAGCGAACGACCTACACCGAACTGAGATACCTACAGCGTGAGCTATGAGA
AAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACG
CCTGGTATCTTTATAGTCCTGTCGGGTTTCGCCACCTCTGACTTGAGCGTC
GATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACATG
TTCTTTCCTGCGTTATCCCCTGATTCTGTGGATAACCGTAGTCGGTCTCAA
ACGCGAACCACCACATTCTCCTTCAATACCTTCCACTAGGTTTTCTATTGC
AGCCTGCATAATAGACATACCTGGCTCCACTTCGGTCTCGATGGTTTCACC
GTCATGCTGAATAAATGTGATTTTTAGCATAGTAACTCCATTATTAATTTT
TAAAATAAGAACTAATAAATTACCTGTTAGTTTGTTTTTTCATATAGTCCG
ATGTAACTAATTTTATAGTCCCACTTGGCTATTTTATTGTTTTAATATTTC
TATATTGTTTTATTTGAATTTTAATCTAATGGAGTTTTAAAAGGTGGAAAA
AGAATTAATAGACTTTATAAATAACGATCTACTCGAAGGTGCTGCTATGTG
AGACCAGACCAATAAAAAACGCCCGGCGGCAACCGAGCGTTCTGAACAAA
TCCAGATGGAGTTCTGAGGTCATTACTGGATCTATCAACAGGAGTCCAAG
CGAGCTCGATATCAAATTACGCCCCGCCCTGCCACTCATCGCAGTACTGTT
GTAATTCATTAAGCATTCTGCCGACATGGAAGCCATCACAAACGGCATGA
TGAACCTGAATCGCCAGCGGCATCAGCACCTTGTCGCCTTGCGTATAATAT
TTGCCCATGGTGAAAACGGGGGCGAAGAAGTIGTCCATATTGGCCACGTT
TAAATCAAAACTGGTGAAACTCACCCAGGGATTGGCTGAAACGAAAAAC
ATATTCTCAATAAACCCTTTAGGGAAATAGGCCAGGTTTTCACCGTAACAC
GCCACATCTTGCGAATATATGTGTAGAAACTGCCGGAAATCGTCGTGGTA
TTCACTCCAGAGCGATGAAAACGTTTCAGTTTGCTCATGGAAAACGGTGT
AACAAGGGTGAACACTATCCCATATCACCAGCTCACCGTCTTTCATTGCCA
TACGAAATTCCGGATGAGCATTCATCAGGCGGGCAAGAATGTGAATAAAG
GCCGGATAAAACTTGTGCTTATTTTTCTTTACGGTCTTTAAAAAGGCCGTA
ATATCCAGCTGAACGGTCTGGTTATAGGTACATTGAGCAACTGACTGAAA
TGCCTCAAAATGTTCTTTACGATGCCATTGGGATATATCAACGGTGGTATA
TCCAGTGATTTTTTTCTCCATTTTAGCTTCCTTAGCTCCTGAAAATCTCGA
TAACTCAAAAAATACGCCCGGTAGTGATCTTATTTCATTATGGTGAAAGTT
GGAACCTCTTACGTGCCCGATCAATCATGACCAAAATCCCTTAAC

The sequence of the exemplary Type-3 coding GFP vector is below. The coding sequence enclosed by BsaI cut sites for modular assembly is indicated by an underline. BsaI restriction site recognition sites are indicated in bold:

(SEQ ID NO: 7)
TCATGACCAAAATCCCTTAACGTGAGTTTTCGTTCCACTGAGCGTCAGACC
CCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGA
GCGCAGATACCAAATACTGTTCTTCTAGTGTAGCCGTAGTTAGGCCACCAC
TTCAAGAACTCTGTAGCACCGCCTACATACCTCGCTCTGCTAATCCTGTTA
CCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGT
TCGTGCACACAGCCCAGCTTGGAGCGAACGACCTACACCGAACTGAGATA
CCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTT
GCTGGCCTTTTGCTCACATGTTCTTTCCTGCGTTATCCCCTGATTCTGTGG
ATAACCGTAGTCGGTCTCATATGAGTAAAGGAGAAGAGCTTTTCACAGGA
GTTGTCCCAATCCTCGTGGAATTAGACGGTGATGTTAATGGGCACAAGTTC
TCTGTCAGTGGAGAGGGTGAAGGCGACGCAACATATGGCAAGCTGACCCT
TAAATTTATTTGCACCACGGGTAAACTACCTGTTCCATGGCCAACACTGGT
CACTACGTTCGGGTATGGGGTTCAGTGCTTTGCGCGCTACCCAGATCACAT
GAAACAGCACGACTTTTTCAAGAGTGCAATGCCCGAAGGCTATGTACAGG
AGAGAACCATCTTTTTTAAGGATGACGGCAACTATAAGACACGCGCCGAA
GTGAAGTTCGAGGGTGATACCCTTGTTAATAGAATCGAGTTAAAGGGTAT
TGACTTTAAGGAAGATGGAAATATTTTAGGCCACAAACTGGAATATAACT
ATAACTCCCATAATGTGTACATTATGGCCGACAAGCAAAAGAACGGTATC
AAGGTTAACTTCAAGATCAGACACAACATTGAGGATGGAAGCGTTCAACT
AGCCGACCATTACCAACAAAACACCCCAATTGGCGATGGGCCTGTGCTGT
TACCAGACAACCATTACCTGTCCACTCAATCTGCCCTTTCGAAAGATCCCA
ACGAAAAGCGCGACCACATGGTCCTTCTTGAGTTTGTCACGGCTGCTGGG
ATTACACACGGCATGGATGAACTATACAAATAAATCCTGAGACCAGACC
AATAAAAAACGCCCGGCGGCAACCGAGCGTTCTGAACAAATCCAGATGG
AGTTCTGAGGTCATTACTGGATCTATCAACAGGAGTCCAAGCGAGCTCGA
TATCAAATTACGCCCCGCCCTGCCACTCATCGCAGTACTGTTGTAATTCAT
TAAGCATTCTGCCGACATGGAAGCCATCACAAACGGCATGATGAACCTGA
ATCGCCAGCGGCATCAGCACCTTGTCGCCTTGCGTATAATATTTGCCCATG
GTGAAAACGGGGGCGAAGAAGTIGTCCATATTGGCCACGTTTAAATCAAA
ACTGGTGAAACTCACCCAGGGATTGGCTGAAACGAAAAACATATTCTCAA
TAAACCCTTTAGGGAAATAGGCCAGGTTTTCACCGTAACACGCCACATCTT
GCGAATATATGTGTAGAAACTGCCGGAAATCGTCGTGGTATTCACTCCAG
AGCGATGAAAACGTTTCAGTTTGCTCATGGAAAACGGTGTAACAAGGGTG
AACACTATCCCATATCACCAGCTCACCGTCTTTCATTGCCATACGAAATTC
CGGATGAGCATTCATCAGGCGGGCAAGAATGTGAATAAAGGCCGGATAA
AACTTGTGCTTATTTTTCTTTACGGTCTTTAAAAAGGCCGTAATATCCAGC
TGAACGGTCTGGTTATAGGTACATTGAGCAACTGACTGAAATGCCTCAAA
ATGTTCTTTACGATGCCATTGGGATATATCAACGGTGGTATATCCAGTGAT
TTTTTTCTCCATTTTAGCTTCCTTAGCTCCTGAAAATCTCGATAACTCAAA
AAATACGCCCGGTAGTGATCTTATTTCATTATGGTGAAAGTTGGAACCTCT
TACGTGCCCGATCAA

The sequence of the exemplary Type-3 coding nanoluc vector is below. The coding sequence enclosed by BsaI cut sites for modular assembly is indicated by an underline. BsaI restriction site recognition sites are indicated in bold:

(SEQ ID NO: 8)
TCATGACCAAAATCCCTTAACGTGAGTTTTCGTTCCACTGAGCGTCAGACC
CCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGA
GCGCAGATACCAAATACTGTTCTTCTAGTGTAGCCGTAGTTAGGCCACCAC
TTCAAGAACTCTGTAGCACCGCCTACATACCTCGCTCTGCTAATCCTGTTA
CCAGTGGCTGCTGCCAGTGGCGATAAGTCGTGTCTTACCGGGTTGGACTC
AAGACGATAGTTACCGGATAAGGCGCAGCGGTCGGGCTGAACGGGGGGT
TCGTGCACACAGCCCAGCTTGGAGCGAACGACCTACACCGAACTGAGATA
CCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTC
GCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTT
GCTGGCCTTTTGCTCACATGTTCTTTCCTGCGTTATCCCCTGATTCTGTGG
ATAACCGTAGTCGGTCTCATATGGTCTTCACACTCGAAGATTTCGTTGGGG
ACTGGCGACAGACAGCCGGCTACAACCTGGACCAAGTCCTTGAACAGGGA
GGTGTGTCCAGTTTGTTTCAGAATCTCGGGGTGTCCGTAACTCCGATCCAA
AGGATTGTCCTGAGCGGTGAAAATGGGCTGAAGATCGACATCCATGTCAT
CATCCCGTATGAAGGTCTGAGCGGCGACCAAATGGGCCAGATCGAAAAA
ATTTTTAAGGTGGTGTACCCTGTGGATGATCATCACTTTAAGGTGATCCTG
CACTATGGCACACTGGTAATCGACGGGGTTACGCCGAACATGATCGACTA
TTTCGGACGGCCGTATGAAGGCATCGCCGTGTTCGACGGCAAAAAGATCA
CTGTAACAGGGACCCTGTGGAACGGCAACAAAATTATCGACGAGCGCCTG
ATCAACCCCGACGGCTCCCTGCTGTTCCGAGTAACCATCAACGGAGTGAC
CGGCTGGCGGCTGTGCGAACGCATTCTGGCGTAAATCCTGAGACCAGACC
AATAAAAAACGCCCGGCGGCAACCGAGCGTTCTGAACAAATCCAGATGG
AGTTCTGAGGTCATTACTGGATCTATCAACAGGAGTCCAAGCGAGCTCGA
TATCAAATTACGCCCCGCCCTGCCACTCATCGCAGTACTGTTGTAATTCAT
TAAGCATTCTGCCGACATGGAAGCCATCACAAACGGCATGATGAACCTGA
ATCGCCAGCGGCATCAGCACCTTGTCGCCTTGCGTATAATATTTGCCCATG
GTGAAAACGGGGGCGAAGAAGTTGTCCATATTGGCCACGTTTAAATCAAA
ACTGGTGAAACTCACCCAGGGATTGGCTGAAACGAAAAACATATTCTCAA
TAAACCCTTTAGGGAAATAGGCCAGGTTTTCACCGTAACACGCCACATCTT
GCGAATATATGTGTAGAAACTGCCGGAAATCGTCGTGGTATTCACTCCAG
AGCGATGAAAACGTTTCAGTTTGCTCATGGAAAACGGTGTAACAAGGGTG
AACACTATCCCATATCACCAGCTCACCGTCTTTCATTGCCATACGAAATTC
CGGATGAGCATTCATCAGGCGGGCAAGAATGTGAATAAAGGCCGGATAA
AACTTGTGCTTATTTTTCTTTACGGTCTTTAAAAAGGCCGTAATATCCAGC
TGAACGGTCTGGTTATAGGTACATTGAGCAACTGACTGAAATGCCTCAAA
ATGTTCTTTACGATGCCATTGGGATATATCAACGGTGGTATATCCAGTGAT
TTTTTTCTCCATTTTAGCTTCCTTAGCTCCTGAAAATCTCGATAACTCAAA
AAATACGCCCGGTAGTGATCTTATTTCATTATGGTGAAAGTTGGAACCTCT
TACGTGCCCGATCAA

The sequence of the exemplary Type-3 coding mRuby vector is below. The coding sequence enclosed by BsaI cut sites for modular assembly is indicated by an underline. BsaI restriction site recognition sites are indicated in bold:

(SEQ ID NO: 9)
TCGGTCTCATATGGTGTCCAAAGGAGAGGAGTTAATCAAGGAAAACATGA
GAATGAAAGTTGTCATGGAGGGCTCCGTTAATGGTCACCAATTCAAGTGT
ACAGGGGAAGGTGAAGGTAATCCTTACATGGGTACACAAACTATGAGAAT
TAAAGTAATTGAAGGCGGACCACTACCATTTGCATTTGACATTCTGGCAA
CGTCATTCATGTACGGATCACGAACTTTCATCAAGTACCCTAAAGGTATAC
CAGACTTTTTCAAGCAATCTTTTCCAGAGGGTTTTACATGGGAAAGGGTTA
CAAGATACGAAGATGGGGGTGTCGTCACAGTTATGCAAGATACTTCATTA
GAAGATGGCTGCCTTGTCTATCATGTGCAAGTAAGAGGGGTGAATTTTCCT
TCTAACGGACCTGTGATGCAGAAAAAGACCAAAGGTTGGGAACCAAATA
CTGAAATGATGTACCCAGCTGATGGAGGTTTGAGAGGCTACACACACATG
GCGCTTAAAGTTGATGGTGGAGGTCATTTGTCTTGTAGTTTTGTTACCACT
TATCGTTCTAAAAAGACTGTTGGCAATATCAAAATGCCAGGAATACATGC
TGTAGACCACAGACTAGAAAGACTCGAAGAGAGCGATAACGAAATGTTC
GTTGTACAGAGAGAGCATGCCGTAGCCAAATTTGCTGGCTTAGGCGGTGG
TATGGATGAATTGTATAAGGGATCCTGAGACCAGACCAATAAAAAACGCC
CGGCGGCAACCGAGCGTTCTGAACAAATCCAGATGGAGTTCTGAGGTCAT
TACTGGATCTATCAACAGGAGTCCAAGCGAGCTCGATATCAAATTACGCC
CCGCCCTGCCACTCATCGCAGTACTGTTGTAATTCATTAAGCATTCTGCCG
ACATGGAAGCCATCACAAACGGCATGATGAACCTGAATCGCCAGCGGCAT
CAGCACCTTGTCGCCTTGCGTATAATATTTGCCCATGGTGAAAACGGGGG
CGAAGAAGTIGTCCATATTGGCCACGTTTAAATCAAAACTGGTGAAACTC
ACCCAGGGATTGGCTGAAACGAAAAACATATTCTCAATAAACCCTTTAGG
GAAATAGGCCAGGTTTTCACCGTAACACGCCACATCTTGCGAATATATGT
GTAGAAACTGCCGGAAATCGTCGTGGTATTCACTCCAGAGCGATGAAAAC
GTTTCAGTTTGCTCATGGAAAACGGTGTAACAAGGGTGAACACTATCCCA
TATCACCAGCTCACCGTCTTTCATTGCCATACGAAATTCCGGATGAGCATT
CATCAGGCGGGCAAGAATGTGAATAAAGGCCGGATAAAACTTGTGCTTAT
TTTTCTTTACGGTCTTTAAAAAGGCCGTAATATCCAGCTGAACGGTCTGGT
TATAGGTACATTGAGCAACTGACTGAAATGCCTCAAAATGTTCTTTACGAT
GCCATTGGGATATATCAACGGTGGTATATCCAGTGATTTTTTTCTCCATTT
TAGCTTCCTTAGCTCCTGAAAATCTCGATAACTCAAAAAATACGCCCGGTA
GTGATCTTATTTCATTATGGTGAAAGTTGGAACCTCTTACGTGCCCGATCA
ATCATGACCAAAATCCCTTAACGTGAGTTTTCGTTCCACTGAGCGTCAGAC
CCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCA
GAGCGCAGATACCAAATACTGTTCTTCTAGTGTAGCCGTAGTTAGGCCAC
CACTTCAAGAACTCTGTAGCACCGCCTACATACCTCGCTCTGCTAATCCTG
TTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGG
GTTCGTGCACACAGCCCAGCTTGGAGCGAACGACCTACACCGAACTGAGA
TACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACG
AGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTT
TGCTGGCCTTTTGCTCACATGTTCTTTCCTGCGTTATCCCCTGATTCTGTG
GATAACCGTAG

The sequence of the exemplary Type-4 terminator vector is below. The terminator sequence enclosed by BsaI cut sites for modular assembly is indicated by an underline. BsaI restriction site recognition sites are indicated in bold:

(SEQ ID NO: 10)
GGTCGGGCTGAACGGGGGGTTCGTGCACACAGCCCAGCTTGGAGCGAACG
ACCTACACCGAACTGAGATACCTACAGCGTGAGCTATGAGAAAGCGCCAC
GCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCT
GCGTTATCCCCTGATTCTGTGGATAACCGTAGTCGGTCTCAATCCTAACTA
GCATAACCCCTTGGGGCCTCTAAACGGGTCTTGAGGGGTTTTTTGCTGGCT
GTGAGACCAGACCAATAAAAAACGCCCGGCGGCAACCGAGCGTTCTGAAC
AAATCCAGATGGAGTTCTGAGGTCATTACTGGATCTATCAACAGGAGTCC
AAGCGAGCTCGATATCAAATTACGCCCCGCCCTGCCACTCATCGCAGTAC
TGTTGTAATTCATTAAGCATTCTGCCGACATGGAAGCCATCACAAACGGC
ATGATGAACCTGAATCGCCAGCGGCATCAGCACCTTGTCGCCTTGCGTAT
AATATTTGCCCATGGTGAAAACGGGGGCGAAGAAGTTGTCCATATTGGCC
ACGTTTAAATCAAAACTGGTGAAACTCACCCAGGGATTGGCTGAAACGAA
AAACATATTCTCAATAAACCCTTTAGGGAAATAGGCCAGGTTTTCACCGT
AACACGCCACATCTTGCGAATATATGTGTAGAAACTGCCGGAAATCGTCG
TGGTATTCACTCCAGAGCGATGAAAACGTTTCAGTTTGCTCATGGAAAAC
GGTGTAACAAGGGTGAACACTATCCCATATCACCAGCTCACCGTCTTTCAT
TGCCATACGAAATTCCGGATGAGCATTCATCAGGCGGGCAAGAATGTGAA
TAAAGGCCGGATAAAACTTGTGCTTATTTTTCTTTACGGTCTTTAAAAAGG
CCGTAATATCCAGCTGAACGGTCTGGTTATAGGTACATTGAGCAACTGAC
TGAAATGCCTCAAAATGTTCTTTACGATGCCATTGGGATATATCAACGGTG
GTATATCCAGTGATTTTTTTCTCCATTTTAGCTTCCTTAGCTCCTGAAAAT
CTCGATAACTCAAAAAATACGCCCGGTAGTGATCTTATTTCATTATGGTGA
AAGTTGGAACCTCTTACGTGCCCGATCAATCATGACCAAAATCCCTTAACG
TGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAA
ACCACCGCTACCAGCGGTGGTTTGTTTGCCGGATCAAGAGCTACCAACTCT
TTTTCCGAAGGTAACTGGCTTCAGCAGAGCGCAGATACCAAATACTGTTCT
TCTAGTGTAGCCGTAGTTAGGCCACCACTTCAAGAACTCTGTAGCACCGCC
TACATACCTCGCTCTGCTAATCCTGTTACCAGTGGCTGCTGCCAGTGGCGA
TAAGTCGTGTCTTACCGGGTTGGACTCAAGACGATAGTTACCGGATAAGG
CGCAGC

The sequence of the exemplary Type-5 linker vector is below. The linker sequence enclosed by BsaI cut sites for modular assembly is indicated by an underline. BsaI restriction site recognition sites are indicated in bold:

(SEQ ID NO: 11)
TCGGTCTCAGCTGGAAATCTGCTCGTCAGTGGTGCTCACACTGACGAATC
ATGTACAGATCATACCGATGACTGCCTGGCGACTCACAACTAAGCAAGAC
AGCCGGAACCAGCGCCGGCGAACACCACTGCATATATGGCATATCACAAC
AGTCCACGTCTCAAGCAGTTACAGAGATGTTACGAACCACTAGTGCACTG
CAGTACATGAGACCAGACCAATAAAAAACGCCCGGCGGCAACCGAGCGT
TCTGAACAAATCCAGATGGAGTTCTGAGGTCATTACTGGATCTATCAACA
GGAGTCCAAGCGAGCTCGATATCAAATTACGCCCCGCCCTGCCACTCATC
GCAGTACTGTTGTAATTCATTAAGCATTCTGCCGACATGGAAGCCATCACA
AACGGCATGATGAACCTGAATCGCCAGCGGCATCAGCACCTTGTCGCCTT
GCGTATAATATTTGCCCATGGTGAAAACGGGGGCGAAGAAGTTGTCCATA
TTGGCCACGTTTAAATCAAAACTGGTGAAACTCACCCAGGGATTGGCTGA
AACGAAAAACATATTCTCAATAAACCCTTTAGGGAAATAGGCCAGGTTTT
CACCGTAACACGCCACATCTTGCGAATATATGTGTAGAAACTGCCGGAAA
TCGTCGTGGTATTCACTCCAGAGCGATGAAAACGTTTCAGTTTGCTCATGG
AAAACGGTGTAACAAGGGTGAACACTATCCCATATCACCAGCTCACCGTC
TTTCATTGCCATACGAAATTCCGGATGAGCATTCATCAGGCGGGCAAGAA
TGTGAATAAAGGCCGGATAAAACTTGTGCTTATTTTTCTTTACGGTCTTTA
AAAAGGCCGTAATATCCAGCTGAACGGTCTGGTTATAGGTACATTGAGCA
ACTGACTGAAATGCCTCAAAATGTTCTTTACGATGCCATTGGGATATATCA
ACGGTGGTATATCCAGTGATTTTTTTCTCCATTTTAGCTTCCTTAGCTCCT
GAAAATCTCGATAACTCAAAAAATACGCCCGGTAGTGATCTTATTTCATTA
TGGTGAAAGTTGGAACCTCTTACGTGCCCGATCAATCATGACCAAAATCCC
TTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAA
TACTGTTCTTCTAGTGTAGCCGTAGTTAGGCCACCACTTCAAGAACTCTGT
AGCACCGCCTACATACCTCGCTCTGCTAATCCTGTTACCAGTGGCTGCTGC
CAGTGGCGATAAGTCGTGTCTTACCGGGTTGGACTCAAGACGATAGTTAC
CGGATAAGGCGCAGCGGTCGGGCTGAACGGGGGGTTCGTGCACACAGCC
CAGCTTGGAGCGAACGACCTACACCGAACTGAGATACCTACAGCGTGAGC
TATGAGAAAGCGCCACGCTTCCCGAAGGGAGAAAGGCGGACAGGTATCC
GGTAAGCGGCAGGGTCGGAACAGGAGAGCGCACGAGGGAGCTTCCAGGG
GGAAACGCCTGGTATCTTTATAGTCCTGTCGGGTTTCGCCACCTCTGACTT
GAGCGTCGATTTTTGTGATGCTCGTCAGGGGGGCGGAGCCTATGGAAAAA
CGCCAGCAACGCGGCCTTTTTACGGTTCCTGGCCTTTTGCTGGCCTTTTGC
TCACATGTTCTTTCCTGCGTTATCCCCTGATTCTGTGGATAACCGTAG

The sequence of the exemplary Type-8 RSF1010 backbone vector is below. The sequence enclosed by BsaI cut sites for modular assembly is indicated by an underline. BsaI restriction site recognition sites are indicated in bold:

(SEQ ID NO: 12)
TTAGAAAAACTCATCGAGCATCAAATGAAACTGCAATTTATTCATATCAG
GATTATCAATACCATATTTTTGAAAAAGCCGTTTCTGTAATGAAGGAGAA
AACTCACCGAGGCAGTTCCATAGGATGGCAAGATCCTGGTATCGGTCTGC
GATTCCGACTCGTCCAACATCAATACAACCTATTAATTTCCCCTCGTCAAA
AATAAGGTTATCAAGTGAGAAATCACCATGAGTGACGACTGAATCCGGTG
AGAATGGCAAAAGCTTATGCATTTCTTTCCAGACTTGTTCAACAGGCCAGC
CATTACGCTCGTCATCAAAATCACTCGCATCAACCAAACCGTTATTCATTC
GTGATTGCGCCTGAGCGAGGCGAAATACGCGATCGCTGTTAAAAGGACAA
TTACAAACAGGAATCGAATGCAACCGGCGCAGGAACACTGCCAGCGCAT
CAACAATATTTTCACCTGAATCAGGATATTCTTCTAATACCTGGAATGCTG
TTTTCCCGGGGATCGCAGTGGTGAGTAACCATGCATCATCAGGAGTACGG
ATAAAATGCTTGATGGTCGGAAGAGGCATAAATTCCGTCAGCCAGTTTAG
TCTGACCATCTCATCTGTAACATCATTGGCAACGCTACCTTTGCCATGTTT
CAGAAACAACTCTGGCGCATCGGGCTTCCCATACAATCGATAGATTGTCG
CACCTGATTGCCCGACATTATCGCGAGCCCATTTATACCCATATAAATCAG
CATCCATGTTGGAATTTAATCGCGGCCTGGAGCAAGACGTTTCCCGTTGAA
TATGGCTCATAACACCCCTTGTATTACTGTTTATGTAAGCAGACAGTTTTA
TTGTTCATGATGATATATTTTTATCTTGTGCAATGTAACATCAGAGATTTT
GAGACACAACGTGGCTTTGTTGAATAAATCGAACTTTTGCTGAGTTGAAGG
ATCAGTCATGACCAAAATCCCTTAACGTGAGTCAGCCTGCCGCCTTGGGC
CGGGTGATGTCGTACTTGCCCGCCGCGAACTCGGTTACCGTCCAGCCCAG
CGCGACCAGCTCCGGCAACGCCTCGCGCACCCGCTTGCGGCGCTTGCGCA
TGGTCGAACCACTGGCCTCTGACGGCCAGACATAGCCGCACAAGGTATCT
ATGGAAGCCTTGCCGGTTTTGCCGGGGTCGATCCAGCCACACAGCCGCTG
GTGCAGCAGGCGGGCGGTTTCGCTGTCCAGCGCCCGCACCTCGTCCATGC
TGATGCGCACATGCTGGCCGCCACCCATGACGGCCTGCGCGATCAAGGGG
TTCAGGGCCACGTACAGGCGCCCGTCCGCCTCGTCGCTGGCGTACTCCGA
CAGCAGCCGAAACCCCTGCCGCTTGCGGCCATTCTGGGCGATGATGGATA
CCTTCCAAAGGCGCTCGATGCAGTCCTGTATGTGCTTGAGCGCCCCACCAC
TATCGACCTCTGCCCCGATTTCCTTTGCCAGCGCCCGATAGCTACCTTTGA
CCACATGGCATTCAGCGGTGACGGCCTCCCACTTGGGTTCCAGGAACAGC
CGGAGCTGCCGTCCGCCTTCGGTCTTGGGTTCCGGGCCAAGCACTAGGCC
ATTAGGCCCAGCCATGGCCACCAGCCCTTGCAGGATGCGCAGATCATCAG
CGCCCAGCGGCTCCGGGCCGCTGAACTCGATCCGCTTGCCGTCGCCGTAG
TCATACGTCACGTCCAGCTTGCTGCGCTTGCGCTCGCCCCGCTTGAGGGCA
CGGAACAGGCCGGGGGCCAGACAGTGCGCCGGGTCGTGCCGGACGTGGC
TGAGGCTGTGCTTGTTCTTAGGCTTCACCACGGGGCACCCCCTTGCTCTTG
CGCTGCCTCTCCAGCACGGCGGGCTTGAGCACCCCGCCGTCATGCCGCCT
GAACCACCGATCAGCGAACGGTGCGCCATAGTTGGCCTTGCTCACACCGA
AGCGGACGAAGAACCGGCGCTGGTCGTCGTCCACACCCCATTCCTCGGCC
TCGGCGCTGGTCATGCTCGACAGGTAGGACTGCCAGCGGATGTTATCGAC
CAGTACCGAGCTGCCCCGGCTGGCCTGCTGCTGGTCGCCTGCGCCCATCAT
GGCCGCGCCCTTGCTGGCATGGTGCAGGAACACGATAGAGCACCCGGTAT
CGGCGGCGATGGCCTCCATGCGACCGATGACCTGGGCCATGGGGCCGCTG
GCGTTTTCTTCCTCGATGTGGAACCGGCGCAGCGTGTCCAGCACCATCAGG
CGGCGGCCCTCGGCGGCGCGCTTGAGGCCGTCGAACCACTCCGGGGCCAT
GATGTTGGGCAGGCTGCCGATCAGCGGCTGGATCAGCAGGCCGTCAGCCA
CGGCTTGCCGTTCCTCGGCGCTGAGGTGCGCCCCAAGGGCGTGCAGGCGG
TGATGAATGGCGGTGGGCGGGTCTTCGGCGGGCAGGTAGATCACCGGGCC
GGTGGGCAGTTCGCCCACCTCCAGCAGATCCGGCCCGCCTGCAATCTGTG
CGGCCAGTTGCAGGGCCAGCATGGATTTACCGGCACCACCGGGCGACACC
AGCGCCCCGACCGTACCGGCCACCATGTTGGGCAAAACGTAGTCCAGCGG
TGGCGGCGCTGCTGCGAACGCCTCCAGAATATTGATAGGCTTATGGGTAG
CCATTGATTGCCTCCTTTGCAGGCAGTTGGTGGTTAGGCGCTGGCGGGGTC
ACTACCCCCGCCCTGCGCCGCTCTGAGTTCTTCCAGGCACTCGCGCAGCGC
CTCGTATTCGTCGTCGGTCAGCCAGAACTTGCGCTGACGCATCCCTTTGGC
CTTCATGCGCTCGGCATATCGCGCTTGGCGTACAGCGTCAGGGCTGGCCA
GCAGGTCGCCGGTCTGCTTGTCCTTTTGGTCTTTCATATCAGTCACCGAGA
AACTTGCCGGGGCCGAAAGGCTTGTCTTCGCGGAACAAGGACAAGGTGCA
GCCGTCAAGGTTAAGGCTGGCCATATCAGCGACTGAAAAGCGGCCAGCCT
CGGCCTTGTTTGACGTATAACCAAAGCCACCGGGCAACCAATAGCCCTTG
TCACTTTTGATCAGGTAGACCGACCCTGAAGCGCTTTTTTCGTATTCCATA
AAACCCCCTTCTGTGCGTGAGTACTCATAGTATAACAGGCGTGAGTACCA
ACGCAAGCACTACATGCTGAAATCTGGCCCGCCCCTGTCCATGCCTCGCT
GGCGGGGTGCCGGTGCCCGTGCCAGCTCGGCCCGCGCAAGCTGGACGCTG
GGCAGACCCATGACCTTGCTGACGGTGCGCTCGATGTAATCCGCTTCGTG
GCCGGGCTTGCGCTCTGCCAGCGCTGGGCTGGCCTCGGCCATGGCCTTGC
CGATTTCCTCGGCACTGCGGCCCCGGCTGGCCAGCTTCTGCGCGGCGATA
AAGTCGCACTTGCTGAGGTCATGACCGAAGCGCTTGACCAGCCCGGCCAT
CTCGCTGCGGTACTCGTCCAGCGCCGTGCGCCGGTGGCGGCTAAGCTGCC
GCTCGGGCAGTTCGAGGCTGGCCAGCCTGCGGGCCTTCTCCTGCTGCCGCT
GGGCCTGCTCGATCTGCTGGCCAGCCTGCTGCACCAGCGCCGGGCCAGCG
GTGGCGGTCTTGCCCTTGGATTCACGCAGCAGCACCCACGGCTGATAACC
GGCGCGGGTGGTGTGCTTGTCCTTGCGGTTGGTGAAGCCCGCCAAGCGGC
CATAGTGGCGGCTGTCGGCGCTGGCCGGGTCGGCGTCGTACTCGCTGGCC
AGCGTCCGGGCAATCTGCCCCCGAAGTTCACCGCCTGCGGCGTCGGCCAC
CTTGACCCATGCCTGATAGTTCTTCGGGCTGGTTTCCACTACCAGGGCAGG
CTCCCGGCCCTCGGCTTTCATGTCATCCAGGTCAAACTCGCTGAGGTCGTC
CACCAGCACCAGACCATGCCGCTCCTGCTCGGCGGGCCTGATATACACGT
CATTGCCCTGGGCATTCATCCGCTTGAGCCATGGCGTGTTCTGGAGCACTT
CGGCGGCTGACCATTCCCGGTTCATCATCTGGCCGGTGGGTGCGTCCCTGA
CGCCGATATCGAAGCGCTCACAGCCCATGGCCTTGAGCTGTCGGCCTATG
GCCTGCAAAGTCCTGTCGTTCTTCATCGGGCCACCAAGCGCAGCCAGATC
GAGCCGTCCTCGGTTGTCAGTGGCGTCAGGTCGAGCAAGAGCAACGATGC
GATCAGCAGCACCACCGTAGGCATCATGGAAGCCAGCATCACGGTTAGCC
ATAGCTTCCAGTGCCACCCCCGCGACGCGCTCCGGGCGCTCTGCGCGGCG
CTGCTCACCTCGGCGGCTACCTCCCGCAACTCTTTGGCCAGCTCCACCCAT
GCCGCCCCTGTCTGGCGCTGGGCTTTCAGCCACTCCGCCGCCTGCGCCTCG
CTGGCCTGCTTGGTCTGGCTCATGACCTGCCGGGCTTCGTCGGCCAGTGTC
GCCATGCTCTGGGCCAGCGGTTCGATCTGCTCCGCTAACTCGTTGATGCCT
CTGGATTTCTTCACTCTGTCGATTGCGTTCATGGTCTATTGCCTCCCGGTA
TTCCTGTAAGTCGATGATCTGGGCGTTGGCGGTGTCGATGTTCAGGGCCAC
GTCTGCCCGGTCGGTGCGGATGCCCCGGCCTTCCATCTCCACCACGTTCGG
CCCCAGGTGAACACCGGGCAGGCGCTCGATGCCCTGCGCCTCAAGTGTTC
TGTGGTCAATGCGGGCGTCGTGGCCAGCCCGCTCTAATGCCCGGTTGGCA
TGGTCGGCCCATGCCTCGCGGGTCTGCTCAAGCCATGCCTTGGGCTTGAGC
GCTTCGGTCTTCTGTGCCCCGCCCTTCTCCGGGGTCTTGCCGTTGTACCGC
TTGAACCACTGAGCGGCGGGCCGCTCGATGCCGTCATTGATCCGCTCGGAG
ATCATCAGGTGGCAGTGCGGGTTCTCGCCGCCACCGGCATGGATGGCCAG
CGTATACGGCAGGCGCTCGGCACCGGTCAGGTGCTGGGCGAACTCGGACG
CCAGCGCCTTCTGCTGGTCGAGGGTCAGCTCGACCGGCAGGGCAAATTCG
ACCTCCTTGAACAGCCGCCCATTGGCGCGTTCATACAGGTCGGCAGCATC
CCAGTAGTCGGCGGGCCGCTCGACGAACTCCGGCATGTGCCCGGATTCGG
CGTGCAAGACTTCATCCATGTCGCGGGCATACTTGCCTTCGCGCTGGATGT
AGTCGGCCTTGGCCCTGGCCGATTGGCCGCCCGACCTGCTGCCGGTTTTCG
CCGTAAGGTGATAAATCGCCATGCTGCCTCGCTGTTGCTTTTGCTTTTCGG
CTCCATGCAATGGCCCTCGGAGAGCGCACCGCCCGAAGGGTGGCCGTTAG
GCCAGTTTCTCGAAGAGAAACCGGTAAGTGCGCCCTCCCCTACAAAGTAG
GGTCGGGATTGCCGCCGCTGTGCCTCCATGATAGCCTACGAGACAGCACA
TTAACAATGGGGTGTCAAGATGGTTAAGGGGAGCAACAAGGCGGCGGAT
CGGCTGGCCAAGCTCGAAGAACAACGAGCGCGAATCAATGCCGAAATTC
AGCGGGAGCGGGCAAGGGAACAGCAGCAAGAGCGCAAGAACGAAACAA
GGCGCAAGGTGCTGGTGGGGGCCATGATTTTGGCCAAGGTGAACAGCAGC
GAGTGGCCGGAGGATCGGCTCATGGCGGCAATGGATGCGTACCTTGAACG
CGACCACGACCGCGCCTTGTTCGGTCTGCCGCCACGCCAGAAGGATGAGC
CGGGCTGAATGATCGACCGAGACAGGCCCTGCGGGGCTGCACACGCGCCC
CCACCCTTCGGGTAGGGGGAAAGGCCGCTAAAGCGGCTAAAAGCGCTCC
AGCGTATTTCTGCGGGGTTTGGTGTGGGGTTTAGCGGGCTTTGCCCGCCTT
TCCCCCTGCCGCGCAGCGGTGGGGCGGTGTGTAGCCTAGCGCAGCGAATA
GACCAGCTATCCGGCCTCTGGCCGGGCATATTGGGCAAGGGCAGCAGCGC
CCCACAAGGGCGCTGATAACCGCGCCTAGTGGATTATTCTTAGATAATCA
TGGATGGATTTTTCCAACACCCCGCCAGCCCCCGCCCCTGCTGGGTTTGCA
GGTTTGGGGGCGTGACAGTTATTGCAGGGGTTCGTGACAGTTATTGCAGG
GGGGCGTGACAGTTATTGCAGGGGTTCGTGACAGTTAGTACGGGAGTGAC
GGGCACTGGCTGGCAATGTCTAGCAACGGCAGGCATTTCGGCTGAGGGTA
AAAGAACTTTCCGCTAAGCGATAGACTGTATGTAAACACAGTATTGCAAG
GACGCGGAACATGCCTCATGTGGCGGCCAGGACGGCCAGCCGGGATCGG
GATACTGGTCGTTACCAGAGCCACCGACCCGAGCAAACCCTTCTCTATCA
GATCGTTGACGAGTATTACCCGGCATTCGCTGCGCTTATGGCAGAGCAGG
GAAAGGAATTGCCGGGCTATGTGCAACGGGAATTTGAAGAATTTCTCCAA
TGCGGGCGGCTGGAGCATGGCTTTCTACGGGTTCGCTGCGAGTCTTGCCAC
GCCGAGCACCTGGTCGCTTTCAGAAATCAATCTAAAGTATATATGAGTAA
ACTTGGTCTGACAGGCCCCTTGAGACCAGTCCCTATCAGTGATAGAGATT
GACATCCCTATCAGTGATAGAGATACTGAGCACGGATCTGAAAGAGGAGA
AAGGATCTATGGCGAGTAGCGAAGACGTTATCAAAGAGTTCATGCGTTTC
AAAGTTCGTATGGAAGGTTCCGTTAACGGTCACGAGTTCGAAATCGAAGG
TGAAGGTGAAGGTCGTCCGTACGAAGGTACTCAGACCGCTAAACTGAAAG
TTACCAAAGGTGGTCCGCTGCCGTTCGCTTGGGACATCCTGTCCCCGCAGT
TCCAGTACGGTTCCAAAGCTTACGTTAAACACCCGGCTGACATCCCGGAC
TACCTGAAACTGTCCTTCCCGGAAGGTTTCAAATGGGAACGTGTTATGAA
CTTCGAAGACGGTGGTGTTGTTACCGTTACCCAGGACTCCTCCCTGCAAGA
CGGTGAGTTCATCTACAAAGTTAAACTGCGTGGTACTAACTTCCCGTCCGA
CGGTCCGGTTATGCAGAAAAAAACCATGGGTTGGGAAGCTTCCACCGAAC
GTATGTACCCGGAAGACGGTGCTCTGAAAGGTGAAATCAAAATGCGTCTG
AAACTGAAAGACGGTGGTCACTACGACGCTGAAGTTAAAACCACCTACAT
GGCTAAAAAACCGGTTCAGCTGCCGGGTGCTTACAAAACCGACATCAAAC
TGGACATCACCTCCCACAACGAAGACTACACCATCGTTGAACAGTACGAA
CGTGCTGAAGGTCGTCACTCCACCGGTGCTTAATAAGGATCTCCAGGCAT
CAAATAAAACGAAAGGCTCAGTCGAAAGACTGGGCCTTTCGTTTTATCTG
TTGTTTGTCGGTGAACGCTCTCTACTAGAGTCACACTGGCTCACCTTCGGG
TGGGCCTTTCTGCGTTTATAAGTCGGTCTCACCGAGCGGCCGCGTGTTAC
AACCAATTAACCAATTCTGA

We show that CRISPR interference (CRISPRi) can be used in Pseudoalteromonas bacteria to knock down secondary metabolite gene expression. With the MMK as provided herein, we quantify and compare the expression from promoters driving genes that are key for host-microbe interactions. We use MMK as provided herein to perform live cell imaging of Roseobacter bacteria present within the gut of the biofouling tubeworm Hydroides elegans, a discovery that has significant implications for the process of bacteria-stimulated metamorphosis.

The genetic manipulation of Pseudoalteromonas sp. PS5 provides a proof-of-concept that we can use genetically modified bacteria to demonstrate the mechanistic effects of probiotic bacteria on coral larvae or adults. The ability to genetically manipulate marine probiotic bacteria allows production of enhanced marine bacterial strains. The genetic manipulation of Pseudoalteromonas sp. PS5 as described herein provides a proof-of-concept modified bacteria can be genetically modified, and then used to test hypotheses about the mechanistic effects of probiotic bacteria on coral larvae or adults. Knowledge gained using such methodologies will help activists make informed choices about risker interventions for coral reef restoration. The ability to genetically manipulate marine probiotic bacteria using products of manufacture and kits, and methods as provided herein opens the door to the production of enhanced marine bacterial strains with the ability to produce stimulatory products that amplify their probiotic effects for reef restoration and biotechnology applications.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Isolation and Characterization of Nereida Alphaproteobacteria Strain MMG025

This example describes the isolation and characterization of a new marine bacterial strain Nereida alphaproteobacteria strain MMG025.

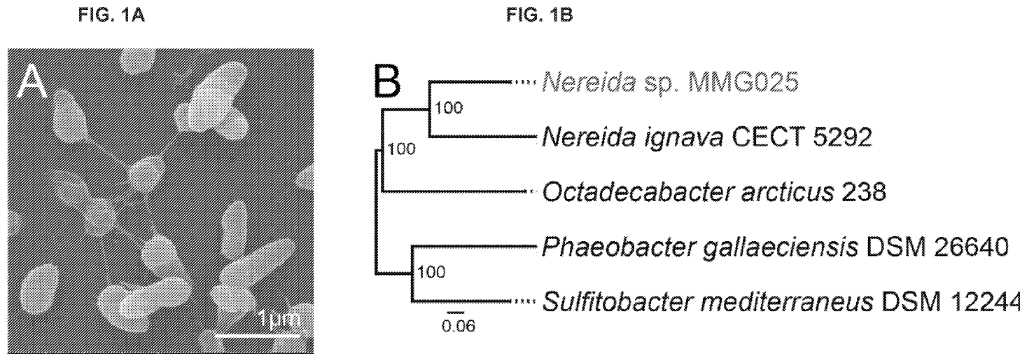

Novel marine bacteria were isolated and cultured, and the genomes were sequenced, assembled, annotated and analyzed. Strain MMG025 was isolated from the surface of a Giant Kelp from the La Jolla tide pools, California, USA (32.8411° N, 117.28170 W) using a sterile cotton swab. A single colony was obtained on Marine Agar 2216 (BD Difco, Franklin Lakes, NJ, USA) and incubated at 28° C. for 72 hours. Colonies were transferred to MARINE BROTH 2216™ (Difco) and incubated for 72 hours at 25° C. before storage, DNA isolation and imaging by scanning electron microscopy, as illustrated in FIG. 1.

FIG. 1. (A) Scanning Electron Micrograph of Nereida sp. MMG025. (B) A maximum likelihood phylogeny constructed using the Codon tree method though the Pathosystems Resource Integration Center (PATRIC) (NIH) using 100 single-copy genes and proteins identified by PGFams (Global Protein Families, in the PATRIC database) (8, 19-25).

Genomic DNA was extracted using a QUICK-DNA FUNGAL/BACTERIAL MINIPREP KIT™ (Zymo Research, Irvine, CA, USA). 16S rRNA gene (27F-1492R) Sanger sequencing (Eton Biosciences, San Diego, CA, USA) identified the closest strain as Nereida ignava CECT 5292 (97.99% ID, 0.0 E-value). DNA was submitted to the Microbial Genome Sequencing Center (Pittsburgh, PA USA) for library preparation (DNA PREP KIT™; Illumina, San Diego, CA, USA) and whole-genome sequencing (NEXTSEQ 550™; Illumina), producing 2×150 base pairs (bp) paired-end reads. Reads were trimmed using TRIM GALORE v.0.6.5™ (1), assembled using UNICYCLER v0.4.8™ (2) integrated in PATRIC v3.6.12™ (3) and annotated using the PROKARYOTIC GENOME ANNOTATION PIPELINE (PGAP) v5.1™ (NCBI) (4), with default parameters. MMG025 has a 3.1-Mb genome, a total GC content of 56% with 40 contigs and an N₅₀ value of 628,545 bp, with 3,260 predicted coding sequences. Default parameters were used except where otherwise noted.

A phylogenetic analysis revealed that strain MMG025 falls into the genus Nereida (FIG. 1), which is part of the Roseobacter group, in the family Rhodobacteraceae and class Alphaproteobacteria (5-7). Comparing strain MMG025 with Nereida ignava CECT 5292 yields an ANI value of 72.47 (3, 8, 9), a distance that is below the 95% threshold that delineates species (10), suggesting that MMG025 is a novel isolate. We designate the current isolate as Nereida sp. strain MMG025.

We found that strain MMG025 harbors a homolog of the DMSP demethylase gene dmdA (80% ID; 100% query cover; e-value 0) (14). Because of their natural occurrence with plants and animals and antagonistic properties against pathogenic bacteria, Roseobacter species are promising candidates for use as probiotics in aquaculture or for environmental restoration (15-18). The isolation and genome sequence of Nereida sp. MMG025 provides a valuable resource for studying the ecology of Roseobacter bacteria and serves as an asset for biotechnology applications.

Data availability. The genome sequencing and assembly project for strain MMG025 has been deposited in DDBJ/EMBL/GenBank under BioProject number PRJNA716944, raw sequencing SRA accession number SRR17607627 and whole-genome sequencing genome accession number JAKFZN000000000.

### Example 2: Functionally Linking Tetrabromopyrrole Genes to Coral Metamorphosis

This example describes genetic techniques to manipulate the bacterium Pseudoalteromonas sp. PS5 to explore tetrabromopyrrole (TBP)-induced metamorphosis in the hard coral Porites astreoides.

In this study, we establish the genetic techniques to manipulate the bacterium, Pseudoalteromonas sp. PS5, to explore TBP-induced metamorphosis in the hard coral Porites astreoides. We find that a deletion of the brominase gene, bmp2, disrupts TBP production in Pseudoalteromonas sp. PS5 and ablates the bacterium's ability to stimulate the metamorphosis of P. astreoides larvae. Our results attribute TBP production from live bacteria to the stimulation of metamorphosis in corals and bring us closer to realizing the use of genetically modified bacteria for studying and improving bacteria for use as coral probiotics.

To test whether Pseudoalteromonas sp. PS5 stimulates coral metamorphosis through the production of TBP, we set out to generate a genetically tractable strain lacking a key bmp biosynthesis gene. We searched the sequenced Pseudoalteromonas sp. PS5 genome [28] and identified the bmp gene cluster (Genbank accession KR011923) by blastn. Using conjugation to deliver a suicide plasmid we performed double homologous recombination resulting in an in-frame deletion that includes the first two and last three amino acids of the bmp2 gene, thus generating a truncated bmp2 knockout strain.

We next quantified the production of TBP from the Pseudoalteromonas sp. PS5 wild type and Δbmp2 strains using LCMS-MS. When grown in liquid media for 24 h, Pseudoalteromonas sp. PS5 produces 1.47 T 0.69 mM TBP in culture while a Δbmp2 mutant is unable to produce TBP (FIG. 1i). When compared to a different Pseudoalteromonas species Pseudoalteromonas luteoviolacea that carries a homologous bmp gene cluster, we find that Pseudoalteromonas sp. PS5 produces 30× more TBP when using the same culturing, extraction and quantification conditions [29]. These results show that removing the bmp2 gene from Pseudoalteromonas sp. PS5 stops TBP production and indicates that bmp2 is the only brominase genes contributing to TBP biosynthesis in Pseudoalteromonas sp. PS5 under the conditions tested.

To determine whether bacteria lacking the ability to biosynthesize TBP are unable to promote coral metamorphosis, we then compared the ability for Pseudoalteromonas sp. PS5 wild type and Δbmp2 strains to stimulate the metamorphosis of Porites astreoides coral larvae. P. asteroides larvae were chosen for this study because they brood larvae predictably and have been used as a model for metamorphosis in previous studies [23, 30]. When exposed to Pseudoalteromonas sp. PS5 wild type, we observed the metamorphosis of coral larvae, both attached to the substrate and floating (FIG. 1C). Our observations are consistent with previous findings showing that purified TBP promotes both floating and attached larvae [20, 22, 23, 25]. In contrast to the wild type, biofilms of the Pseudoalteromonas sp. PS5 Δbmp2 strain exhibited a significantly reduced ability to stimulate the metamorphosis of coral larvae. However, we did observe a small amount of metamorphosis even when coral larvae were exposed to the Δbmp2 strain. Our results suggest that the stimulatory effect of PS5 on coral metamorphosis is predominantly due to the production of TBP.

Our results establish the functional link between the presence of the bmp biosynthesis genes and the induction of coral metamorphosis by Pseudoalteromonas sp. PS5. While TBP may not be an ecologically relevant inducer of metamorphosis [25], strains that produce TBP may still be useful as probiotics. TBP was shown to have specificity towards the induction of metamorphosis in corals, while not eliciting robust metamorphosis in two other types of invertebrate larvae [29]. The mechanism of action and effect of causing both attached and unattached coral recruits may have significant implications for its usage as a probiotic, especially considering the evidence that TBP elicits phytoplankton mortality [31] and halts sea urchin development [32]. While many questions remain regarding how TBP induces metamorphosis in corals [33], the establishment of new genetic tools both on the bacteria side (this study) and the animal side (Cleves et al. 2018) will enable future studies aimed to determine the mechanism by which TBP induces metamorphosis.

The genetic manipulation of Pseudoalteromonas sp. PS5 provides a proof-of-concept that scientists can use genetically modified bacteria to test hypotheses about the mechanistic effects of probiotic bacteria on coral larvae or adults. Knowledge gained using such methodologies may ultimately help activists make informed choices about risker interventions for coral reef restoration. The ability to genetically manipulate probiotic bacteria opens the door to the production of enhanced strains with the ability to produce stimulatory products that amplify their probiotic effects for reef restoration and biotechnology applications.

### Materials and Methods

### Bacterial Strains and Growth Conditions

Bacterial strains and plasmids used in this study are described herein.

Pseudoalteromonas sp. PS5 was cultured with natural seawater tryptone media NSWT (1 liter (L) natural seawater, 2.5 grams (g)/L Bacto Tryptone, 1.5 μg/L Bacto Yeast and 1.5 ml/L glycerol) and incubated between 25° C. to 28° C. E. coli were grown in LB media and cultured at 37° C. All liquid cultures were inoculated with a single colony and incubated between 14 to 18 hours while shaking at 200 rpm unless otherwise indicated. Plasmids were selected and maintained on LB Kanamycin 100 μg/mL.

### Cloning and Generation of Mutant Strains

Primers used to generated strains in this study are described herein. The in-frame deletion was generated following a previously published protocol [24]. Briefly, Gibson primers were ordered from integrated DNA technologies (IDT) and were designed to amplify 1400 base pair homology arms up and downstream of the bmp2 gene in Pseudoalteromonas sp. PS5. The homology arms were amplified using a high-fidelity DNA polymerase (Primestar, TaKaRa) and the resulting fragments were purified using a DNA Clean and Concentrator kit (Zymo Research). The suicide vector pCVD443 (Huang and Hadfield, 2011) was digested with Sph1, XbaI and SacI. To assemble the digested plasmid and the PCR products, a three fragment Gibson Assembly was performed using the NEBUILDER HIFI DNA ASSEMBLY MASTER MIX™ at a ratio of 2:1 for inserts:backbone vector. Resulting assemblies were diluted and electroporated into SM10 pir electrocompetent cells and selections were performed on LB Kanamycin 100 μg/mL. Clones were PCR screened using p443_F and p443R and positive clones containing a band around 3000 base pairs were cultured, miniprepped using the ZIPPY DNA MINIPREP KIT™ (Zymo Research). Minipreps of the positive clones sent for were confirmed by Sanger sequencing (Eton Biosciences). The pCVD443_PS5Δbmp2 plasmid was conjugated with PS5 according to a previously published double homologous recombination protocol [24]. Selections were performed on NSWT Streptomycin/Kanamycin 200 μg/mL and counter selections were performed on NSWT+10% sucrose.

### Coral Collection and Culturing

Reproductively mature colonies of Porites astreoides were collected after the new moon via SCUBA by the Mote Marine Laboratory (Summerland Key, FL) in June 2021. Coral colonies were placed in a flow through table and larvae were collected in bowls over the course of the night. Larvae were maintained in filtered natural seawater until use in experiments. Larvae selected for experiments were actively swimming.

### Metamorphosis Assay Methods

Wildtype and mutant strains were struck out onto MB media and incubated overnight at 28° C. The next day, single colonies were inoculated into 2 mL culture and incubated with agitation at for 18 hours. The optical density of the cultures were measured and standardized to OD 0.5. Ceramic fragging disks (Aquarium world) were sterilized by autoclave and placed into each well of a sterile, untreated 6-well plate (Falcon). 5 mL of Marine Broth followed by 100 μL of diluted culture was inoculated into each well of the 6-well plate. The plates were then incubated at 28 C for 48 hours with slow agitation (approximately 50 rpm). The biofilmed disks were removed from the wells and rinsed under a steady stream of 0.2 filtered seawater to eliminate unattached cells. Biofilmed disks were then placed into 6 replicate deep petri dishes (Falcon) containing 60 mL of 0.2 FSW. 10 larvae were added to each petri dish in 10 mL, bringing the final volume of the petri dishes to 70 mL. N=6. FIG. 1 represents one biological replicate.

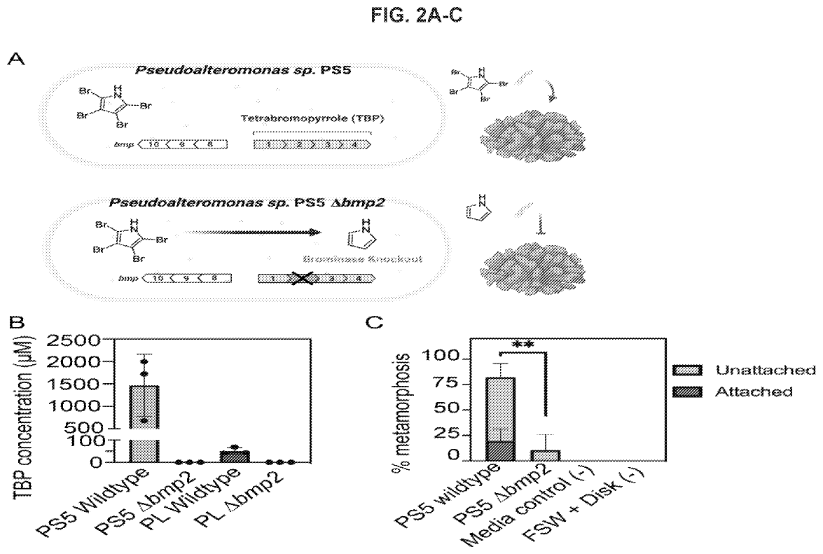

FIG. 2. a Pseudoalteromonas sp. PS5 Strain Lacking the Brominase Gene Bmp2 is Unable to Produce TBP and does not Induce Coral Metamorphosis.

FIG. 2(A) A model of the TBP biosynthesis gene cluster in Pseudoalteromonas sp. PS5 including bmp1-4 and bmp8-10 genes. The bmp2 brominase gene is highlighted in red (also designated “2”).

FIG. 2(B) Pseudoalteromonas sp. PS5 produces 30-fold more TBP than P. luteoviolacea strain HI1 and mutation of the bmp2 gene ablates TBP production in both strains. Error bars represent standard deviation of the mean in plots B-C.

FIG. 2(C) Metamorphosis (%) of Porites astreoides larvae in response to Pseudoalteromonas sp. PS5 wild type and Δbmp2 strains. MARINE BROTH 2216™ (Difco) growth media or Filtered Sea Water (FSW) alone are included as controls.

Statistical significance was determined by a two-tailed Mann Whitney test where p=0.0079. N=6, 10 larvae/well.

### HPLC Methods

Quantification of TBP was performed as previously described [29]. Briefly, replicate cultures of PS5 were grown in 5 mL overnight for 16 hours and extracted with 2× volume of ethyl acetate. LC/MS-MS was performed on all extracts.

### Statistics

Data was plotted and analyzed using PRISM V9™ (Graphpad). Nonparametric statistics were performed on all data. The statistics for the biofilm metamorphosis assays were performed on the combined morphogenesis phenotype (attached and unattached) and a 2-tailed Mann Whitney test was performed to compare PS5 wildtype and PS5Δbmp2 strains (p=0.0079).

### Example 3: New Transposon Vectors Designated Tn7 and Tn10

This example describes new transposon vectors (designated Tn7 and Tn10) that are compatible with the standardized genetic parts system and can stably integrate into the genome of a marine bacterium such as a Pseudoalteromonas and/or a Roseobacter species.

The Tn7 and Tn10 vectors are different because:
(1) Tn7 and Tn10 vectors allow for integration of the plasmid machinery into the chromosome of the recipient bacterium, in contrast to replication of the plasmid machinery separately from the bacterial chromosome;
(2) Tn7 and Tn10 vector integration allows the integrated machinery to persist within the bacterium for multiple generations as opposed to being lost as a plasmid; and
(3) Tn7 and Tn10 vector integration also means that antibiotics are not required to be administered to the bacterium to keep the integrated machinery within the bacterial cell, as opposed to being lost because there is no antibiotic pressure to keep the plasmids.

The nucleic acid sequence of the Tn7 vector is below. The Type-8 Tn7 plasmid sequence is indicated by an underline. The BsaI restriction recognition sites are indicated in bold. The regions of the plasmid that integrate into the bacterial genome are indicated with a strikethrough:

(SEQ ID NO: 1)
TTGGTGTATCCAACGGCGTCAGCCGGGCAGGATAGGTGAAGTAGG
CCCACCCGCGAGCGGGTGTTCCTTCTTCACTGTCCCTTATTCGCACCTGGC
GGTGCTCAACGGGAATCCTGCTCTGCGAGGCTGGCCGGCTACCGCCGGCG
TAACAGATGAGGGCAAGCGGATGGCTGATGAAACCAAGCCAACCAGGAA
GGGCAGCCCACCTATCAAGGTGTACTGCCTTCCAGACGAACGAAGAGCGA
TTGAGGAAAAGGCGGCGGCGGCCGGCATGAGCCTGTCGGCCTACCTGCTG
GCCGTCGGCCAGGGCTACAAAATCACGGGCGTCGTGGACTATGAGCACGT
CCGCGAGCTGGCCCGCATCAATGGCGACCTGGGCCGCCTGGGCGGCCTGC
TGAAACTCTGGCTCACCGACGACCCGCGCACGGCGCGGTTCGGTGATGCC
ACGATCCTCGCCCTGCTGGCGAAGATCGAAGAGAAGCAGGACGAGCTTGG
CAAGGTCATGATGGGCGTGGTCCGCCCGAGGGCAGAGCCATGACTTTTTT
AGCCGCTAAAACGGCCGGGGGGTGCGCGTGATTGCCAAGCACGTCCCCAT
GCGCTCCATCAAGAAGAGCGACTTCGCGGAGCTGGTGAAGTACATCACCG
ACGAGCAAGGCAAGACCGAGCGCCTGGGTCACGTGCGCGTCACGAACTG
CGAGGCAAACACCCTGCCCGCTGTCATGGCCGAGGTGATGGCGACCCAGC
ACGGCAACACCCGTTCCGAGGCCGACAAGACCTATCACCTGCTGGTTAGC
TTCCGCGCGGGAGAGAAGCCCGACGCGGAGACGTTGCGCGCGATTGAGG
ACCGCATCTGCGCTGGGCTTGGCTTCGCCGAGCATCAGCGCGTCAGTGCC
GTGCATCACGACACCGACAACCTGCACATCCATATCGCCATCAACAAGAT
TCACCCGACCCGAAACACCATCCATGAGCCGTATCGGGCCTACCGCGCCC
TCGCTGACCTCTGCGCGACGCTCGAACGGGACTACGGGCTTGAGCGTGAC
AATCACGAAACGCGGCAGCGCGTTTCCGAGAACCGCGCGAACGACATGG
AGCGGCACGCGGGCGTGGAAAGCCTGGTCGGCTGGATCCGGCCACGATG
CGTCCGGCGTAGAGGATCTGAAGATCAGCAGTTCAACCTGTTGATAGTAC
GTACTAAGCTCTCATGTTTCACGTACTAAGCTCTCATGTTTAACGTACTAA
GCTCTCATGTTTAACGAACTAAACCCTCATGGCTAACGTACTAAGCTCTCA
TGGCTAACGTACTAAGCTCTCATGTTTCACGTACTAAGCTCTCATGTTTGA
ACAATAAAATTAATATAAATCAGCAACTTAAATAGCCTCTAAGGTTTTAA
GTTTTATAAGAAAAAAAAGAATATATAAGGCTTTTAAAGCTTTTAAGGTTT
AACGGTTGTGGACAACAAGCCAGGGATGTAACGCACTGAGAAGCCCTTAG
AGCCTCTCAAAGCAATTTTGAGTGACACAGGAACACTTAACGGCTGACAT
GGGAATTCCACATGTGGAATTCCACATGTGGAATTGTGAGCGGATAACAA
TTTGTGGAATTCCCGGGAGAGCTCGATCGGCCGAAGCAGGGGGGCAAGGC
TGAAAAGCCGGCCCCCGCTGCGGCCCCGACCGGCTTCACCTTCAACCCAA
CACCGGACAAAAAGGATCCTCTAGAGGACCAGCCGCGTAACCTGGCAAA
ATCGGTTACGGTTGAGTAATAAATGGATGCCCTGCGTAAGCGGGTGTGGG
CGGACAATAAAGTCTTAAACTGAACAAAATAGATCTAAACTATGACAATA
AAGTCTTAAACTAGACAGAATAGTTGTAAACTGAAATCAGTCCAGTTATG
CTGTGAAAAAGCATACTGGACTTTTGTTATGGCTAAAGCAAACTCTTCATT
TTCTGAAGTGCAAATTGCCCGTCGTATTAAAGAGGGGCGTGGGGTCGAAA
TTCCCGGGGATCCACGCGTCTTAAGGCGGCCTTGCAGTTTCATTTGATGCT
CGATGAGTTTTTCTAATCAGAATTGGTTAATTGGTTGTAACACGCGGCCGC
TCGGTGAGACCGACTTATAAACGCAGAAAGGCCCACCCGAAGGTGAGCC
AGTGTGACTCTAGTAGAGAGCGTTCACCGACAAACAACAGATAAAACGA
AAGGCCCAGTCTTTCGACTGAGCCTTTCGTTTTATTTGATGCCTGGAGATC
CTTATTAAGCACCGGTGGAGTGACGACCTTCAGCACGTTCGTACTGTTCAA
CGATGGTGTAGTCTTCGTTGTGGGAGGTGATGTCCAGTTTGATGTCGGTTT
TGTAAGCACCCGGCAGCTGAACCGGTTTTTTAGCCATGTAGGTGGTTTTAA
CTTCAGCGTCGTAGTGACCACCGTCTTTCAGTTTCAGACGCATTTTGATTT
CACCTTTCAGAGCACCGTCTTCCGGGTACATACGTTCGGTGGAAGCTTCCC
AACCCATGGTTTTTTTCTGCATAACCGGACCGTCGGACGGGAAGTTAGTAC
CACGCAGTTTAACTTTGTAGATGAACTCACCGTCTTGCAGGGAGGAGTCCT
GGGTAACGGTAACAACACCACCGTCTTCGAAGTTCATAACACGTTCCCAT
TTGAAACCTTCCGGGAAGGACAGTTTCAGGTAGTCCGGGATGTCAGCCGG
GTGTTTAACGTAAGCTTTGGAACCGTACTGGAACTGCGGGGACAGGATGT
CCCAAGCGAACGGCAGCGGACCACCTTTGGTAACTTTCAGTTTAGCGGTC
TGAGTACCTTCGTACGGACGACCTTCACCTTCACCTTCGATTTCGAACTCG
TGACCGTTAACGGAACCTTCCATACGAACTTTGAAACGCATGAACTCTTTG
ATAACGTCTTCGCTACTCGCCATAGATCCTTTCTCCTCTTTCAGATCCGTG
CTCAGTATCTCTATCACTGATAGGGATGTCAATCTCTATCACTGATAGGGA
CTGGTCTCAAGGGGCCTGTCAGACCAAGTTTACTCATATATACTTTAGATT
GATTTCTGAAAGCGACCAGGTGCTCGCGGCCGCGGTACCGGGCCCGTCGG
GATCCGGTGATTGATTGAGCAAGCTTTATGCTTGTAAACCGTTTTGTGAAA
AAATTTTTAAAATAAAAAAGGGGACCTCTAGGGTCCCCAATTAGAATTGG
CCGCGGCGTTGTGACAATTTACCGAACAACTCCGCGGCCGGGAAGCCGAT
CTCGGCTTGAACGAATTGTTAGGTGGCGGTACTTGGGTCGATATCAAAGT
GCATCACTTCTTCCCGTATGCCCAACTTTGTATAGAGAGCCACTGCGGGAT
CGTCACCGTAATCTGCTTGCACGTAGATCACATAAGCACCAAGCGCGTTG
GCCTCATGCTTGAGGAGATTGATGAGCGCGGTGGCAATGCCCTGCCTCCG
GTGCTCGCCGGAGACTGCGAGATCATAGATATAGATCTCACTACGCGGCT
GCTCAAACTTGGGCAGAACGTAAGCCGCGAGAGCGCCAACAACCGCTTCT
TGGTCGAAGGCAGCAAGCGCGATGAATGTCTTACTACGGAGCAAGTTCCC
GAGGTAATCGGAGTCCGGCTGATGTTGGGAGTAGGTGGCTACGTCTCCGA
ACTCACGACCGAAAAGATCAAGAGCAGCCCGCATGGATTTGACTTGGTCA
GGGCCGAGCCTACATGTGCGAATGATGCCCATACTTGAGCCACCTAACTT
TGTTTTAGGGCGACTGCCCTGCTGCGTAACATCGTTGCTGCTGCGTAACAT
CGTTGCTGCTCCATAACATCAAACATCGACCCACGGCGTAACGCGCTTGC
TGCTTGGATGCCCGAGGCATAGACTGTACAAAAAAACAGTCATAACAAGC
CATGAAAACCGCCACTGCGCCGTTACCACCGCTGCGTTCGGTCAAGGTTC
TGGACCAGTTGCGTGAGCGCATACGCTACTTGCATTACAGTTTACGAACC
GAACAGGCTTATGTCAATTCGTAATTGGGGACCCTAGAGGTCCCCTTTTTT
ATTTTAAAAATTTTTTCACAAAACGGTTTACAAGCATAAAGCTTGCTCAAT
CAATCACCGGATCCCCGACTCTAGTCGACCTGCAGGCCAACCAGATAAGT
GAAATCTAGTTCCAAACTATTTTGTCATTTTTAATTTTCGTATTAGCTTAC
GACGCTACACCCAGTTCCCATCTATTTTGTCACTCTTCCCTAAATAATCCT
TAAAAACTCCATTTCCACCCCTCCCAGTTCCCAACTATTTTGTCCGCCCAC
AGCGGGGCATTTTTCTTCCTGTTATGTTTGGGCGAGCTCGAATTCACTGGC
CGTCGTTTTACAACGTCGTGACTGGGAAAACCCTGGCGTTACCCAACTTAA
TCGCCTTGCAGCACATCCCCCTTTCGCCAGCTGGCGTAATAGCGAAGAGGC
CCGCACCGATCGCCCTTCCCAACAGTTGCGCAGCCTATCGCATGCGGTACC
TCTAGAAGAAGCTTGGGATCCGTCGACCTGCAGATCTGCAGGTGGCACTTT
TCGGGGAAATGTGCGCGGAACCCCTATTTGTTTATTTTTCTAAATACATTC
AAATATGTATCCGCTCATGAGACAATAACCCTGATAAATGCTTCAATAAT
ATTGAAAAAGGAAGAGTATGAGTATTCAACATTTCCGTGTCGCCCTTATTC
CCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCG
AACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTA
TCCCGTGTTGACGCCGGGCAAGAGCAACTCGGTCGCCGCATACACTATTC
TCAGAATGACTTGGTTGAGTACTCACCAGTCACAGAAAAGCATCTTACGG
ATGGCATGACAGTAAGAGAATTATGCAGTGCTGCCATAACCATGAGTGAT
AACACTGCGGCCAACTTACTTCTGACAACGATCGGAGGACCGAAGGAGCT
AACCGCTTTTTTGCACAACATGGGGGATCATGTAACTCGCCTTGATCGTTG
GGAACCGGAGCTGAATGAAGCCATACCAAACGACGAGCGTGACACCACG
ATGCCTGCAGCAATGGCAACAACGTTGCGCAAACTATTAACTGGCGAACT
ACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCA
CTGGGGCCAGATGGTAAGCCCTCCCGTATCGTAGTTATCTACACGACGGG
GAGTCAGGCAACTATGGATGAACGAAATAGACAGATCGCTGAGATAGGT
GCCTCACTGATTAAGCATTGGTAACTGTCAGACCAAGTTTACTCATATATA
CTTTAGATTGATTTATGGTGCACTCTCAGTACAATCTGCTCTGATGCCGCA
TAGTTAAGCCAGTATACACTCCGCTATCGCTACGTGACTGGGTCATGGCTG
CGCCCCGACACCCGCCAACACCCGCTGACGCGCCCTGACGGGCTTGTCTG
CTCCCGGCATCCGCTTACAGACAAGCTGTGACCGTCTCCGGGAGCTGCAT
GTGTCAGAGGTTTTCACCGTCATCACCGAAACGCGCGAGGCAGCAAGGAG
ATGGCGCCCAACAGTCCCCCGGCCACGGGGCCTGCCACCATACCCACGCC
GAAACAAGCGCTCATGAGCCCGAAGTGGCGAGCCCGATCTTCCCCATCGG
TGATGTCGGCGATATAGGCGCCAGCAACCGCACCTGTGGCGCCGGTGATG
CCGGCCACGATGCGTCCGGCGTAGAGGATCCTTTTTGTCCGGTGTTGGGTT
GAAGGTGAAGCCGGTCGGGGCCGCAGCGGGGGCCGGCTTTTCAGCCTTGC
CCCCCTGCTTCGGCCGCCGTGGCTCCGGCGTCTTGGGTGCCGGCGCGGGTT
CCGCAGCCTTGGCCTGCGGTGCGGGCACATCGGCGGGCTTGGCCTTGATG
TGCCGCCTGGCGTGCGAGCGGAACGTCTCGTAGGAGAACTTGACCTTCCC
CGTTTCCCGCATGTGCTCCCAAATGGTGACGAGCGCATAGCCGGACGCTA
ACGCCGCCTCGACATCCGCCCTCACCGCCAGGAACGCAACCGCAGCCTCA
TCACGCCGGCGCTTCTTGGCCGCGCGGGATTCAACCCACTCGGCCAGCTC
GTCGGTGTAGCTCTTTGGCATCGTCTCTCGCCTGTCCCCTCAGTTCAGTAA
TTTCCTGCATTTGCCTGTTTCCAGTCGGTAGATATTCCACAAAACAGCAGG
GAAGCAGCGCTTTTCCGCTGCATAACCCTGCTTCGGGGTCATTATAGCGAT
TTTTTCGGTATATCCATCCTTTTTCGCACGATATACAGGATTTTGCCAAAG
GGTTCGTGTAGACTTTCC

The nucleic acid sequence of the Tn10 vector is below. The Type-8 Tn10 plasmid sequence is indicated by an underline. The BsaI restriction recognition sites are indicated in bold. The Tn10 inverted repeats are indicated with a strikethrough:

(SEQ ID NO: 2)
CTGCACTAATGTTCCGGCGTTATTTCTTGATGTCTCTGACCAGACACCCAT
CAACAGTATTATTTTCTCCCATGAAGACGGTACGCGACTGGGCGTGGAGC
ATCTGGTCGCATTGGGTCACCAGCAAATCGCGCTGTTAGCGGGCCCATTA
AGTTCTGTCTCGGCGCGTCTGCGTCTGGCTGGCTGGCATAAATATCTCACT
CGCAATCAAATTCAGCCGATAGCGGAACGGGAAGGCGACTGGAGTGCCA
TGTCCGGTTTTCAACAAACCATGCAAATGCTGAATGAGGGCATCGTTCCC
ACTGCGATGCTGGTTGCCAACGATCAGATGGCGCTGGGCGCAATGCGCGC
CATTACCGAGTCCGGGCTGCGCGTTGGTGCGGATATCTCGGTAGTGGGAT
ACGACGATACCGAAGACAGCTCATGTTATATCCCGCCGTTAACCACCATC
AAACAGGATTTTCGCCTGCTGGGGCAAACCAGCGTGGACCGCTTGCTGCA
ACTCTCTCAGGGCCAGGCGGTGAAGGGCAATCAGCTGTTGCCCGTCTCAC
TGGTGAAAAGAAAAACCACCCTGGCGCCCAATACGCAAACCGCCTCTCCC
CGCGCGTTGGCCGATTCATTAATGCAGCTGGCACGACAGGTTTCCCGACT
GGAAAGCGGGCAGTGAGCGCAACGCAATTAATGTAAGTTAGCTCACTCAT
TAGGCACCCCAGGCTTTACACTTTATGCTTCCGACCTGCAGATCTGCAGGT
GGCACTTTTCGGGGAAATGTGCGCGGAACCCCTATTTGTTTATTTTTCTAA
ATACATTCAAATATGTATCCGCTCATGAGACAATAACCCTGATAAATGCTT
CAATAATATTGAAAAAGGAAGAGTATGAGTATTCAACATTTCCGTGTCGC
CCTTATTCCCTTTTTTGCGGCATTTTGCCTTCCTGTTTTTGCTCACCCAGA
AACGCTGGTGAAAGTAAAAGATGCTGAAGATCAGTTGGGTGCACGAGTGG
GTTACATCGAACTGGATCTCAACAGCGGTAAGATCCTTGAGAGTTTTCGCC
CCGAAGAACGTTTTCCAATGATGAGCACTTTTAAAGTTCTGCTATGTGGCG
CGGTATTATCCCGTGTTGACGCCGGGCAAGAGCAACTCGGTCGCCGCATA
CACTATTCTCAGAATGACTTGGTTGAGTACTCACCAGTCACAGAAAAGCA
TCTTACGGATGGCATGACAGTAAGAGAATTATGCAGTGCTGCCATAACCA
TGAGTGATAACACTGCGGCCAACTTACTTCTGACAACGATCGGAGGACCG
AAGGAGCTAACCGCTTTTTTGCACAACATGGGGGATCATGTAACTCGCCTT
GATCGTTGGGAACCGGAGCTGAATGAAGCCATACCAAACGACGAGCGTG
ACACCACGATGCCTGCAGCAATGGCAACAACGTTGCGCAAACTATTAACT
GGCGAACTACTTACTCTAGCTTCCCGGCAACAATTAATAGACTGGATGGA
GGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATC
ATTGCAGCACTGGGGCCAGATGGTAAGCCCTCCCGTATCGTAGTTATCTAC
ACGACGGGGAGTCAGGCAACTATGGATGAACGAAATAGACAGATCGCTG
AGATAGGTGCCTCACTGATTAAGCATTGGTAACTGTCAGACCAAGTTTACT
CATATATACTTTAGATTGATTTATGGTGCACTCTCAGTACAATCTGCTCTG
ATGCCGCATAGTTAAGCCAGTATACACTCCGCTATCGCTACGTGACTGGGT
CATGGCTGCGCCCCGACACCCGCCAACACCCGCTGACGCGCCCTGACGGG
CTTGTCTGCTCCCGGCATCCGCTTACAGACAAGCTGTGACCGTCTCCGGGA
GCTGCATGTGTCAGAGGTTTTCACCGTCATCACCGAAACGCGCGAGGCAG
CAAGGAGATGGCGCCCAACAGTCCCCCGGCCACGGGGCCTGCCACCATAC
CCACGCCGAAACAAGCGCTCATGAGCCCGAAGTGGCGAGCCCGATCTTCC
CCATCGGTGATGTCGGCGATATAGGCGCCAGCAACCGCACCTGTGGCGCC
GGTGATGCCGGCCACGATGCGTCCGGCGTAGAGGATCCTTTTTGTCCGGT
GTTGGGTTGAAGGTGAAGCCGGTCGGGGCCGCAGCGGGGGCCGGCTTTTC
AGCCTTGCCCCCCTGCTTCGGCCGCCGTGGCTCCGGCGTCTTGGGTGCCGG
CGCGGGTTCCGCAGCCTTGGCCTGCGGTGCGGGCACATCGGCGGGCTTGG
CCTTGATGTGCCGCCTGGCGTGCGAGCGGAACGTCTCGTAGGAGAACTTG
ACCTTCCCCGTTTCCCGCATGTGCTCCCAAATGGTGACGAGCGCATAGCCG
GACGCTAACGCCGCCTCGACATCCGCCCTCACCGCCAGGAACGCAACCGC
AGCCTCATCACGCCGGCGCTTCTTGGCCGCGCGGGATTCAACCCACTCGG
CCAGCTCGTCGGTGTAGCTCTTTGGCATCGTCTCTCGCCTGTCCCCTCAGT
TCAGTAATTTCCTGCATTTGCCTGTTTCCAGTCGGTAGATATTCCACAAAA
CAGCAGGGAAGCAGCGCTTTTCCGCTGCATAACCCTGCTTCGGGGTCATTA
TAGCGATTTTTTCGGTATATCCATCCTTTTTCGCACGATATACAGGATTTT
GCCAAAGGGTTCGTGTAGACTTTCCTTGGTGTATCCAACGGCGTCAGCCGG
GCAGGATAGGTGAAGTAGGCCCACCCGCGAGCGGGTGTTCCTTCTTCACTG
TCCCTTATTCGCACCTGGCGGTGCTCAACGGGAATCCTGCTCTGCGAGGCT
GGCCGGCTACCGCCGGCGTAACAGATGAGGGCAAGCGGATGGCTGATGA
AACCAAGCCAACCAGGAAGGGCAGCCCACCTATCAAGGTGTACTGCCTTC
CAGACGAACGAAGAGCGATTGAGGAAAAGGCGGCGGCGGCCGGCATGAG
CCTGTCGGCCTACCTGCTGGCCGTCGGCCAGGGCTACAAAATCACGGGCG
TCGTGGACTATGAGCACGTCCGCGAGCTGGCCCGCATCAATGGCGACCTG
GGCCGCCTGGGCGGCCTGCTGAAACTCTGGCTCACCGACGACCCGCGCAC
GGCGCGGTTCGGTGATGCCACGATCCTCGCCCTGCTGGCGAAGATCGAAG
AGAAGCAGGACGAGCTTGGCAAGGTCATGATGGGCGTGGTCCGCCCGAG
GGCAGAGCCATGACTTTTTTAGCCGCTAAAACGGCCGGGGGGTGCGCGTG
ATTGCCAAGCACGTCCCCATGCGCTCCATCAAGAAGAGCGACTTCGCGGA
GCTGGTGAAGTACATCACCGACGAGCAAGGCAAGACCGAGCGCCTGGGT
CACGTGCGCGTCACGAACTGCGAGGCAAACACCCTGCCCGCTGTCATGGC
CGAGGTGATGGCGACCCAGCACGGCAACACCCGTTCCGAGGCCGACAAG
ACCTATCACCTGCTGGTTAGCTTCCGCGCGGGAGAGAAGCCCGACGCGGA
GACGTTGCGCGCGATTGAGGACCGCATCTGCGCTGGGCTTGGCTTCGCCG
AGCATCAGCGCGTCAGTGCCGTGCATCACGACACCGACAACCTGCACATC
CATATCGCCATCAACAAGATTCACCCGACCCGAAACACCATCCATGAGCC
GTATCGGGCCTACCGCGCCCTCGCTGACCTCTGCGCGACGCTCGAACGGG
ACTACGGGCTTGAGCGTGACAATCACGAAACGCGGCAGCGCGTTTCCGAG
AACCGCGCGAACGACATGGAGCGGCACGCGGGCGTGGAAAGCCTGGTCG
GCTGGATCCGGCCACGATGCGTCCGGCGTAGAGGATCTGAAGATCAGCAG
TTCAACCTGTTGATAGTACGTACTAAGCTCTCATGTTTCACGTACTAAGCT
CTCATGTTTAACGTACTAAGCTCTCATGTTTAACGAACTAAACCCTCATGG
CTAACGTACTAAGCTCTCATGGCTAACGTACTAAGCTCTCATGTTTCACGT
ACTAAGCTCTCATGTTTGAACAATAAAATTAATATAAATCAGCAACTTAA
ATAGCCTCTAAGGTTTTAAGTTTTATAAGAAAAAAAGAATATATAAGGCT
TTTAAAGCTTTTAAGGTTTAACGGTTGTGGACAACAAGCCAGGGATGTAA
CGCACTGAGAAGCCCTTAGAGCCTCTCAAAGCAATTTTGAGTGACACAGG
AACACTTAACGGCTGACATGGGAATTCGGTATACATCACTTTATTTAAAAC
GATGCCCATTTTGTTGATTATTTATTTTTCAGCGCAATTGATAGGCCAAAT
TCCCGCAACGGTGTGGGTGCTATTTACCGAAAATCGTTTTGGATGGAATA
GCATGATGGTTGGCTTTTCATTAGCGGGTCTTGGTCTTTTACACTCAGTAT
TCCAAGCCTTTGTGGCAGGAAGAATAGCCACTAAATGGGGCGAAAAAACG
GCAGTACTGCTCGGATTTATTGCAGATAGTAGTGCATTTGCCTTTTTAGCG
TTTATATCTGAAGGTTGGTTAGTTTTCCCTGTTTTAATTTTATTGGCTGGT
GGTGGGATCGCTTTACCTGCATTACAGGGAGTGATGTCTATCCAAACAAAG
AGTCATCAGCAAGGTGCTTTACAGGGATTATTGGTGAGCCTTACCAATGC
AACCGGTGTTATTGGCCCATTACTGTTTGCTGTTATTTATAATCATTCACT
ACCAATTTGGGATGGCTGGATTTGGATTATTGGTTTAGCGTTTTACTGTAT
TATTATCCTGCTATCGATGACCTTCATGTTAACCCCTCAAGCTCAGGGGAG
TAAACAGGAGACAAGTGCTTAGTTATTTCGTCACCAAATGATGTTATTCCG
CGAAATATAATGACCCTCTTGATAACCCAAGAGGGCATTTTTTACGATAAA
GAAGATTTAGCTTCAAATAAAACCTATCTATTTTATTTATCTTTCAAGCTC
AATAAAAAGCCGCGGTAAATAGCAATAAATTGGCCTTTTTTATCGGCAAG
CTCTTTTAGGTTTTTCGCATGTATTGCGATATGCATAAACCAGCCATTGAG
TAAGTTTTTAAGCACATCATCATCATAAGCTTTCCTGACGGAATGTTAATT
CTCGTTGACCCTGAGCA

[symbol]

[symbol]

[symbol]

[symbol]

CCGGCTGTA
ATCCGGGCAGCGCAACGGAACATTCATCAGTGTAAAAATGGAATCAATAA
AGCCCTGCGCAGCGCGCAGGGTCAGCCTGAATACGCGTGGCCGCCTAGGC
CGCGGCCGCCGGGCAAGTACGACATCACCCGGCCCAAGGCGGCAGGCTG
ACTCACGTTAAGGGATTTTGGTCATGACTGATCCTTCAACTCAGCAAAAGT
TCGATTTATTCAACAAAGCCACGTTGTGTCTCAAAATCTCTGATGTTACAT
TGCACAAGATAAAAATATATCATCATGAACAATAAAACTGTCTGCTTACA
TAAACAGTAATACAAGGGGTGTTATGAGCCATATTCAACGGGAAACGTCT
TGCTCCAGGCCGCGATTAAATTCCAACATGGATGCTGATTTATATGGGTAT
AAATGGGCTCGCGATAATGTCGGGCAATCAGGTGCGACAATCTATCGATT
GTATGGGAAGCCCGATGCGCCAGAGTTGTTTCTGAAACATGGCAAAGGTA
GCGTTGCCAATGATGTTACAGATGAGATGGTCAGACTAAACTGGCTGACG
GAATTTATGCCTCTTCCGACCATCAAGCATTTTATCCGTACTCCTGATGAT
GCATGGTTACTCACCACTGCGATCCCCGGGAAAACAGCATTCCAGGTATT
AGAAGAATATCCTGATTCAGGTGAAAATATTGTTGATGCGCTGGCAGTGT
TCCTGCGCCGGTTGCATTCGATTCCTGTTTGTAATTGTCCTTTTAACAGCG
ATCGCGTATTTCGCCTCGCTCAGGCGCAATCACGAATGAATAACGGTTTGG
TTGATGCGAGTGATTTTGATGACGAGCGTAATGGCTGGCCTGTTGAACAAG
TCTGGAAAGAAATGCATAAGCTTTTGCCATTCTCACCGGATTCAGTCGTCA
CTCATGGTGATTTCTCACTTGATAACCTTATTTTTGACGAGGGGAAATTAA
TAGGTTGTATTGATGTTGGACGAGTCGGAATCGCAGACCGATACCAGGAT
CTTGCCATCCTATGGAACTGCCTCGGTGAGTTTTCTCCTTCATTACAGAAA
CGGCTTTTTCAAAAATATGGTATTGATAATCCTGATATGAATAAATTGCAG
TTTCATTTGATGCTCGATGAGTTTTTCTAATCAGAATTGGTTAATTGGTTG
TAACACGCGGCCGCTCGGTGAGACCGACTTATAAACGCAGAAAGGCCCAC
CCGAAGGTGAGCCAGTGTGACTCTAGTAGAGAGCGTTCACCGACAAACAA
CAGATAAAACGAAAGGCCCAGTCTTTCGACTGAGCCTTTCGTTTTATTTGA
TGCCTGGAGATCCTTATTAAGCACCGGTGGAGTGACGACCTTCAGCACGT
TCGTACTGTTCAACGATGGTGTAGTCTTCGTTGTGGGAGGTGATGTCCAGT
TTGATGTCGGTTTTGTAAGCACCCGGCAGCTGAACCGGTTTTTTAGCCATG
TAGGTGGTTTTAACTTCAGCGTCGTAGTGACCACCGTCTTTCAGTTTCAGA
CGCATTTTGATTTCACCTTTCAGAGCACCGTCTTCCGGGTACATACGTTCG
GTGGAAGCTTCCCAACCCATGGTTTTTTTCTGCATAACCGGACCGTCGGAC
GGGAAGTTAGTACCACGCAGTTTAACTTTGTAGATGAACTCACCGTCTTGC
AGGGAGGAGTCCTGGGTAACGGTAACAACACCACCGTCTTCGAAGTTCAT
AACACGTTCCCATTTGAAACCTTCCGGGAAGGACAGTTTCAGGTAGTCCG
GGATGTCAGCCGGGTGTTTAACGTAAGCTTTGGAACCGTACTGGAACTGC
GGGGACAGGATGTCCCAAGCGAACGGCAGCGGACCACCTTTGGTAACTTT
CAGTTTAGCGGTCTGAGTACCTTCGTACGGACGACCTTCACCTTCACCTTC
GATTTCGAACTCGTGACCGTTAACGGAACCTTCCATACGAACTTTGAAAC
GCATGAACTCTTTGATAACGTCTTCGCTACTCGCCATAGATCCTTTCTCCT
CTTTCAGATCCGTGCTCAGTATCTCTATCACTGATAGGGATGTCAATCTCT
ATCACTGATAGGGACTGGTCTCAAGGGGCCTGTCAGACCAAGTTTACTCAT
ATATACTTTAGATTGATTTCTGAAAGCGACCAGGTGCTCGCGGCCGCACG
CGTATTCAGGCTGACCCTGCGCGCTGCGCAGGGCTTTATTGATTCCATTTT
TACACTGATGAATGTTCCGTTGCGCTGCCCGGATTACAGCCGGATCCGGG

[symbol]

[symbol]

[symbol]

[symbol]

TGCTCAGGGTCAACGAGAATTAACATTCCGTC
AGGAAAGCTTTAAGTTGGTTCTCTTGGATCAATTTGCTGACAATGGCGTTT
ACCTTACCAGTAATGTATTCAAGGCTAATTTTTTCAAGTTCATTCCAACCA
ATGATAGGCATCACTTCTTGGATAGGGATAAGGTTTTTATTATTATCAATA
ATATAATCAAGATAATGTTCAAATATACTTTCTAAGGCAGACCAACCATTT
GTTAAATCAGTTTTTGTTGTGATGTAGGCATCAATCATAATTAATTGCTGC
TTATAACAGGCACTGAGTAATTGTTTTTTATTTTTAAAGTGATGATAAAAG
GCACCTTTGGTCACCAACGCTTTTCCCGAGATCGATCTCATCTATTGAAAC
AGCTTGATAGCCTTTTTCAACAAACAATATTCGTGCTGAGTTAACCAGTGA
TTGATAGGTACTCTTAAAATTTTCTTGTTGATGATTTTTATTTTCCATGAT
AGATTTAAAATAACATACCGTCAGTATGTTTATGGTATCATGATGATGTGG
TCGTGACAATCTTAAGAACATTTAGGTTATTTTATGTATATTGAACAGCAT
TCTCGCTATCAAAATAAAGCTAATAACATCCAATTAAGATATGATGATAAG
CAGTTTCATACAACGGTTATCAAAGATGTTCTATTATGGATTGAACATAAT
TTAGATCAGTCTTTACTGCTTGATGATGTGGCGAATAAAGCGGGTTATACC
AAGTGGTATTTTCAGCGGCTGTTCAAAAAGTAACAGGGGTCACACTGGCT
AGCTATATTCGTGCTCGTCGTTTGACGAAAGCGGCTGTTGAGTTGAGGTTG
ACGAAAAAAACTATCCTTGAGATCGCATTAAAATATCAATTTGATTCCCA
ACAATCTTTTACACGTCGATTTAAGTACATTTTTAAGGTTACACCAAGTTA
TTATCGGCGTAATAAATTATGGGAATTGGAGGCAATGCACTGAGAGATCC
CCTCATAATTTCCCCAAAGCGTAACCATGTGTGAATAAATTTTGAGCTAGT
AGGGTTGCAGCCACGAGTAAGTCTTCCCTTGTTATTGTGTAGCCAGAATGC
CGCAAAACTTCCATGCCTAAGCGAACTGTTGAGAGTACGTTTCGATTTCTG
ACTGTGTTAGCCTGGAAGTGCTTGTCCCAACCTTGTTTCTGAGCATGAACG
CCCGCAAGCCAACATGTTAGITGAAGCATCAGGGCGATTAGCAGCATGAT
ATCAAAACGCTCTGAGCTGCTCGTTCGGCTATGGCGTAGGCCTAGTCCGTA
GGCAGGACTTTTCAAGTCTCGGAAGGTTTCTTCAATCTGCATTCGCTTCGA
ATAGATATTAACAAGTTGTTTGGGTGTTCGAATTTCAACAGGTAAGTTAGT
TGCTAGAACCCATGGCTCCTTTGCCGACGCTGAGTAGATTTTAGGTGACGG
GTGGTGACAATGAGTCCGTGTCGAGCGCTGATTTTTTCGGCCTTTAGAGCG
AGATTTATACAATAGAATTTGGCATGAGATTGGATTGCTTTTAGTCAGCCT
CTTATAGCCTAAAGTCTTTGAGTGACTAGATGACATATCATGTAAGTTGCT
GATAGGTTTCCAGTTTTCCGCTCCTAGGTCTGCATATTGTACTTTTCCTCT
TACTCGACTTAACCAGTACCAACCCAGCTTCTCAACGGATTTATACCATGG
CACTTTAAAGCCAGCATCACTGACAATGAGCGGTGTGGTGTTACTCGGTAG
AATGCTCGCAAGGTCGGCTAGAAATTGGTCATGAGCTTTCTTTGAACATTG
CTCTGAAAGCGGGAACGCTTTCTCATAAAGAGTAACAGAACGACCGTGTA
GTGCGACTGAAGCTCGCAATACCATAAGTCGTTTTTGCTCACGAATATCAG
ACCAGTCAACAAGTACAATGGGCATCGTATTGCCCGAACAGATAAAGCTA
GCATGCCAACGGTATACAGCGAGTCGCTCTTTGTGGAGGTGACGATTACC
TAACAATCGGTCGATTCGTTTGATGTTATGTTTTGTTCTCGCTTTGGTTGG
CAGGTTACGGCCAAGTTCGGTAAGAGTGAGAGTTTTACAGTCAAGTAATGC
GTGGCAAGCCAACGTTAAGCTGTTGAGTCGTTTTAAGTGTAATTCGGGGC
AGAATTGGTAAAGAGAGTCGTGTAAAATATCGAGTTCGCACATCTTGTTG
TCTGATTATTGATTTTTCGCGAAACCATTTGATCCTGTTTCCTGTGTGAAA
TTGTTATCCGCTCACAATTCCACACATTATACGAGCCGATGATTAATTGTC
AACAGCTCATTTCAGAATATTTGCCAGAACCGTTATGATGTCGGCGCAAAA
AACATTATCCAGAACGGGAGTGCGCCTTGAGCGACACGAATTATGCAGTG
ATTTACGACCTGCACAGCCATACCACAGCTTCCGATGGCTGCCTGACGCC
AGAAGCATTGGTGCACCGTGCAGTCGATGATAAGCTGTCAAACATGAGAA
TTCCCGGGAGAGCTCGGTACCCGACACCATCGAATGGTGCAAAACCTTTC
GCGGTATGGCATGATAGCGCCCGGAAGAGAGTCAATTCAGGGTGGTGAAT
GTGAAACCAGTAACGTTATACGATGTCGCAGAGTATGCCGGTGTCTCTTAT
CAGACCGTTTCCCGCGTGGTGAACCAGGCCAGCCACGTTTCTGCGAAAAC
GCGGGAAAAAGTGGAAGCGGCGATGGCGGAGCTGAATTACATTCCCAAC
CGCGTGGCACAACAACTGGCGGGCAAACAGTCGTTGCTGATTGGCGTTGC
CACCTCCAGTCTGGCCCTGCACGCGCCGTCGCAAATTGTCGCGGCGATTA
AATCTCGCGCCGATCAACTGGGTGCCAGCGTGGTGGTGTCGATGGTAGAA
CGAAGCGGCGTCGAAGCCTGTAAAGCGGCGGTGCACAATCTTCTCGCGCA
ACGCGTCAGTGGGCTGATCATTAACTATCCGCTGGATGACCAGGATGCCA
TTGCTGTGGAAGCTGC

### Example 4: A Modular Plasmid Toolkit for the Genetic Engineering of Diverse Marine Bacteria

This Example describes a novel composition or product of manufacture, a Marine Modification Kit (MMK), for genetically modifying (engineering) marine bacteria, particularly those which before this invention remained genetically intractable.

Marine bacteria play significant roles in symbiotic and ecosystem-level processes in the sea. Although harnessing the genetic power of these microbes could open numerous avenues for biotechnology, aquaculture and environmental restoration, many marine bacteria remain genetically intractable.

A significant bottleneck in genetically modifying marine bacteria from diverse lineages is the broad variation in natural antibiotic resistances and genetic avenues that different bacteria require to stably replicate or integrate foreign DNA. A tangible solution to this problem is to rapidly create and iteratively test potential genetic modification strategies, which would allow for the identification of targeted tools that fit the requirements of specific species.

We describe a Marine Modification Kit (MMK) to streamline a mix-and-match workflow to genetically modify marine bacteria. Specifically, we adapt existing and add new standardized genetic parts plasmids that can be assembled by GOLDEN GATE ASSEMBLY™ (Thermo Fisher Scientific, Waltham, MA) that facilitate fluorescent tagging, luminescence detection, transposon-mediated chromosomal integration and CRISPR interference (CRISPRi) capabilities in species from the Roseobacter, Pseudoalteromonas and Vibrio genuses.

To demonstrate the MMK's utility for studying host-microbe interactions, we perform live cell imaging during and after the stimulation of the metamorphic development of a marine invertebrate host.

The MMK as described herein provides a strategy for unlocking our ability to genetically engineer diverse marine microbes, opening significant avenues for fundamental research and biotechnology applications in previously intractable marine microbes.

We describe a Marine Modification Kit (MMK) that adds to and modifies the standardized genetic parts from prominent platforms like the Yeast Tool Kit (YTK) and Bee Tool Kit (BTK) for use in a diversity of marine bacteria. We demonstrate the functionality of the MMK in marine bacteria that perform important symbiotic functions with marine plants or animals. Specifically, we demonstrate that a number of previously tractable and intractable Roseobacter, Pseudoalteromonas and Vibrio species stably carry broad host range plasmids and express fluorescent proteins and nanoluciferase genes.

Provided herein are new transposon vectors (designated Tn7 and Tn10, see Example 3) that are compatible with the standardized genetic parts system and stably integrate into the genome of marine Pseudoalteromonas and Roseobacter species.

We show that CRISPR interference (CRISPRi) can be used in Pseudoalteromonas bacteria to knock down secondary metabolite gene expression. With the MMK, we quantify and compare the expression from promoters driving genes that are key for host-microbe interactions. Finally, we use the MMG to perform live cell imaging of Roseobacter bacteria present within the gut of the biofouling tubeworm Hydroides elegans, a discovery that has significant implications for the process of bacteria-stimulated metamorphosis.

### Results and Discussion

### a Modular Plasmid Toolkit for Genetic Engineering of Marine Bacteria.

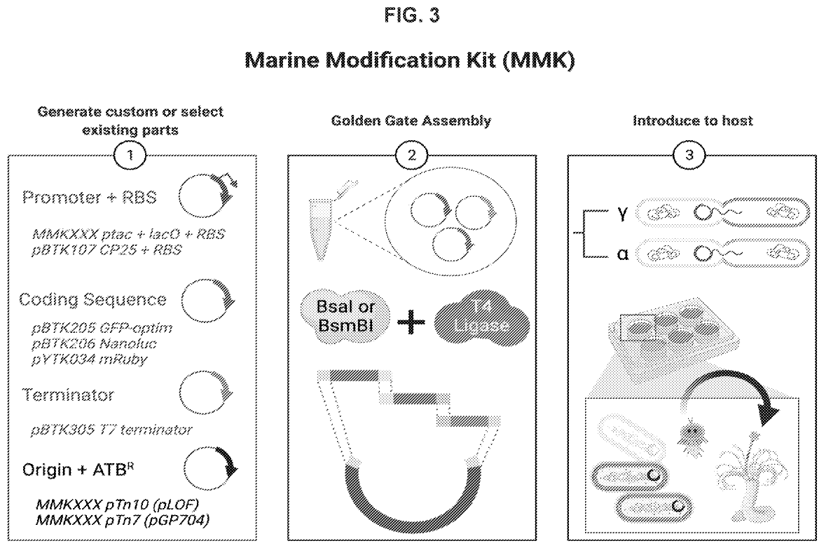

The exemplary MMK system as provided herein utilizes and builds upon the standardized parts and GOLDEN GATE ASSEMBLY™ (Thermo Fisher Scientific, Waltham, MA) principles from BTK and YTK platforms, allowing integration with parts available from both toolkits, as schematically illustrated in FIG. 3 (a schematic overview of the exemplary modular MMK system and integration into diverse bacteria for experiment testing). Stage 0 plasmids include Type-1 and Type-5 connector parts for Stage 2 assembly, a Type-2 promoter part with ribosome binding site (RBS), Type-3 protein coding sequence (CDS) part, Type-4 terminator part, Type-6 repressor part, Type-7 promoter with RBS part and a Type-8 backbone part. Standardized Stage 0 parts are combined into Stage 1 plasmids via GOLDEN GATE ASSEMBLY™ (Thermo Fisher Scientific, Waltham, MA) to create a functional unit. Multiple stage 1 plasmids may be combined together into Stage 2 plasmids as described in BTK and YTK systems (CITE). Importantly for the purposes of engineering diverse marine bacteria, specific parts with antibiotic resistance markers, origins of replication or integrative transposons may be combined into Stage 1 or Stage 2 plasmids that are compatible with the target marine bacterium based on natural properties such as antibiotic susceptibility or propensity to receive transposon insertions.

While this example focuses on the genetic engineering of marine bacteria, the MMK system (for example, the products of manufacture) as provided herein can be applied to a range of bacteria in or outside of the ocean as long as they are amenable to genetic transformation.

### Diverse Marine Bacteria Stably Replicate Stage 1 Plasmids and Express Fluorescent Proteins.

To determine whether Pseudoalteromonas, Roseobacter and Vibrio species are amenable to genetic manipulation using a standardized molecular cloning system, we first screened for natural antibiotic susceptibility to three commonly used antibiotics (kanamycin, gentamycin, streptomycin). We observed that many marine bacteria are susceptible to at least one of the antibiotics tested and might therefore be amenable to antibiotic selection after conjugation of modular plasmids.

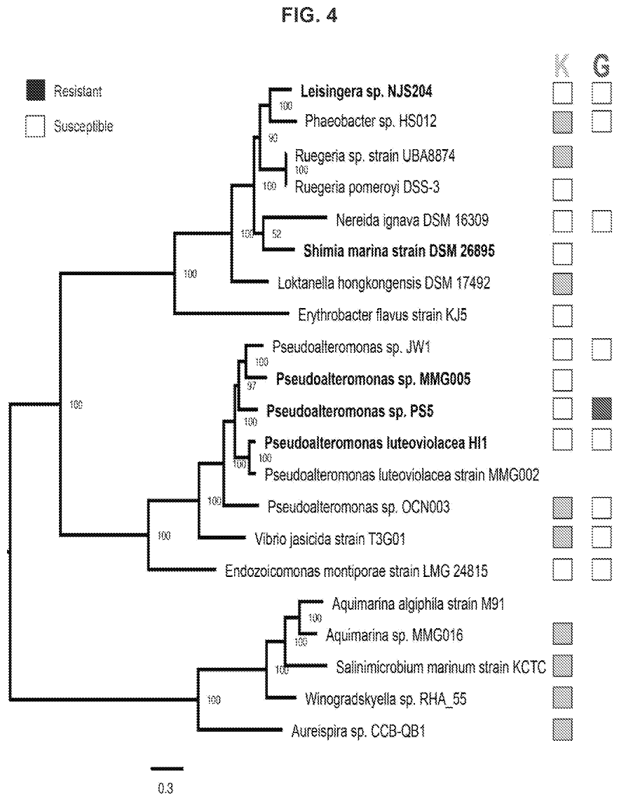

Type-8 origin of replication parts from the BTK system utilize broad host range plasmids containing an RSF1010 origin of replication for the conjugative transfer and stable replication into the marine bacteria of interest. To test whether Pseudoalteromonas, Roseobacter and Vibrio species are amenable to conjugation and stable replication of existing BTK and YTK plasmids, we assembled Stage 1 plasmids comprised of a Type-2 broad host range CP25 promoter, Type-3 GFP or mRuby protein coding sequence (CDS), Type-4 terminator, Type-1 and Type-5 connector parts, with the Type-8 RSF1010 backbone. These Stage 1 plasmids were tested for their ability to be conjugated into a set of marine species. We observed conjugation and fluorescent protein expression in numerous Pseudoalteromonas, Roseobacter and Vibrio species, as illustrated in FIG. 4.

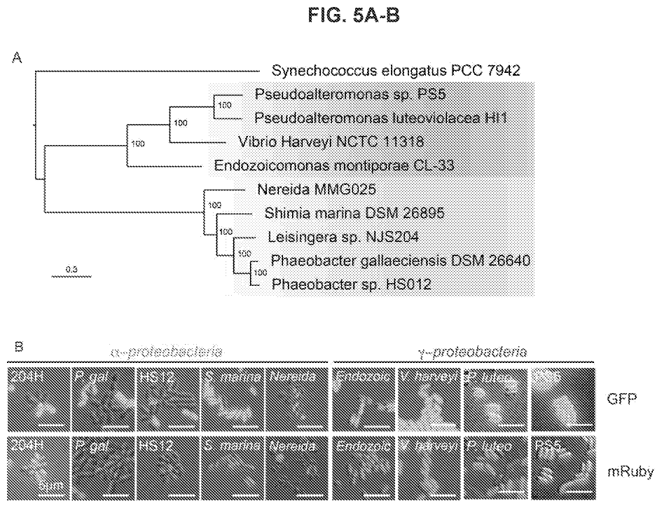

FIG. 5B illustrates images of Pseudoalteromonas, Roseobacter and Vibrio species cells having stage-1 plasmids that are stably replicated and express fluorescent proteins.

Tn7 and Tn10 Transposon Backbone Parts Allow for Integration of MMK Plasmids into Marine Bacterial Genomes.

While stable replication of plasmids in marine bacteria can be useful under some experimental conditions, the retention of plasmids within the bacterial strains often requires the constant presence of antibiotics. One way to circumvent the need for antibiotics to maintain plasmids is by using transposons to insert genetic elements into the bacterial genome. We reasoned that coupling transposons with a standardized molecular cloning system could provide a powerful means to modify marine bacteria for use in experimental or applied systems where antibiotic selection is not desirable or feasible.

To integrate transposon functionality into the MMK system, we created Type-8 mini-Tn7 (pMMK) and Type-8 Tn10 (pMMK) parts. These Type-8 transposon parts were assembled into Stage-1 plasmids containing a constitutively expressed gfp CDS. Because Pseudoalteromonas are amenable to Tn10 mutagenesis, we created P. luteoviolacea and Pseudoalteromonas PS5 strains constitutively expressing gfp via a Tn10 insertion. Similarly, we created a Roseobacter species, P. gallaeciensis, and a Vibrio species, V harveyi, that constitutively expressed gfp via a Tn7 insertion.

CRISPRi Knockdown of Secondary Metabolite Expression in Pseudoalteromonas luteoviolacea.

Pseudoalteromonas species are known for their ability to produce diverse secondary metabolites. To demonstrate the utility of the MMK system in studying marine bacteria, we tested whether Pseudoalteromonas luteoviolacea is susceptible to CRISPR interference (CRISPRi) using standardized molecular cloning technique.

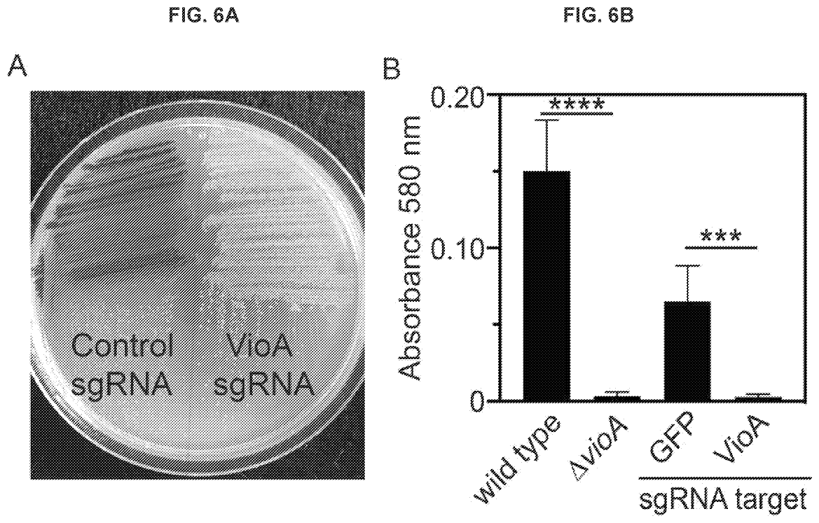

FIG. 6A-B illustrate CRISPRi reduces gene expression in Pseudoalteromonas:

FIG. 6A) Agar plate of P. luteoviolacea comparing the control (sgRNA-GFP) to the violacein knockdown (sgRNA-VioA5).

FIG. 6B) Quantification of Violacein extracted from overnight cultures of P. luteoviolacea containing a gfp control sgRNA plasmid versus an sgRNA targeting VioA. Absorbance was measured at 580 nm. N=8. Error bars represent standard deviation. Statistical significance was determined by a Brown-Forsythe and Welch's ANOVA with Dunnetts multiple comparisons.

### MMK Plasmids Allow for Live Cell Measurements of Natural Product Gene Expression.

We next set out to demonstrate the utility of MMK plasmids in quantifying gene expression in live marine bacteria. Pseudoalteromonas species are known to produce two products that stimulate the metamorphosis of marine animals, Metamorphosis Associated Contractile structures (MACs) and tetrabromopyrrole (TBP). However, the expression of genes responsible for producing these products under different growth and environmental conditions has not yet been explored.

To quantify gene expression of MACs and TBP genes, we created Type-2 promoter part plasmids containing the promoters from the major baseplate gene of MACs, macB, and the TBP operon, starting with bmp1. Type-2 promoters were then combined into Stage-1 plasmids by GOLDEN GATE ASSEMBLY™ (Thermo Fisher Scientific, Waltham, MA) with the nanoluciferase (nanoluc) CDS and into the Type-8 Tn10 transposon backbone, which are known to integrate into Pseudoalteromonas bacterial genomes.

Bacteria that Promote Tubeworm Metamorphosis are Present within the Gut of Juvenile Animals.

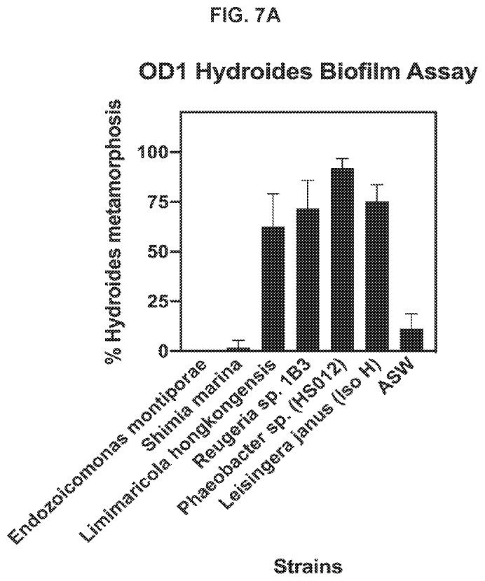

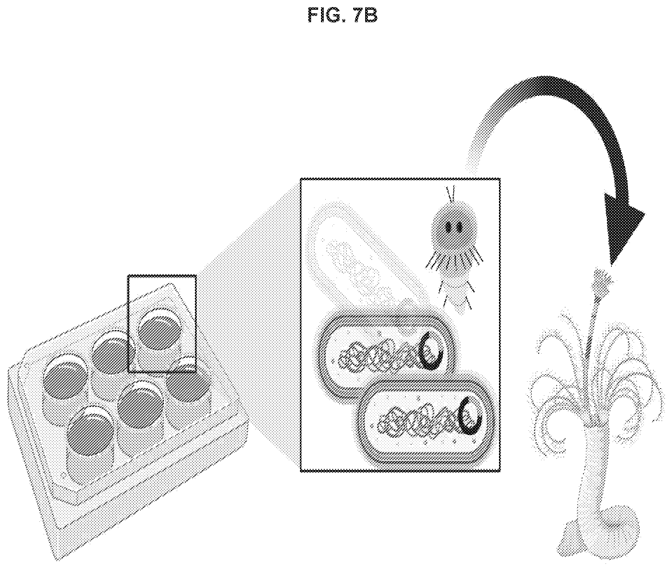

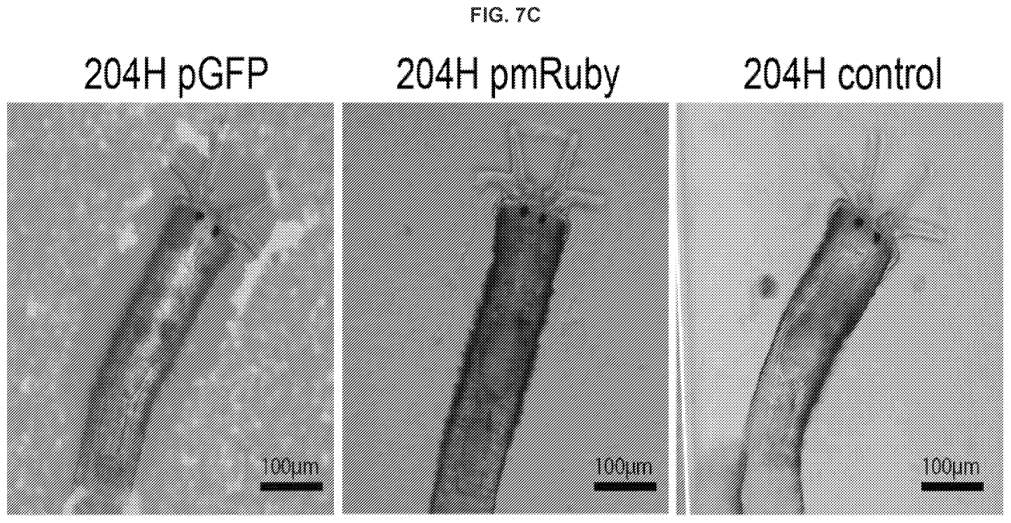

To demonstrate the utility of the MMK platform in host-microbe interactions, we tested whether a set of marine bacteria with MMK plasmids expressing fluorescent proteins would be able to stimulate the metamorphosis of the tubeworm Hydroides elegans. Indeed, Pseudoalteromonas luteoviolacea strain HI1 has been previously demonstrated to stimulate the metamorphosis of Hydroides), and was able to stimulate metamorphosis while carrying the MMK plasmid. Additionally, two Roseobacter species that have not previously been shown to stimulate animal metamorphosis, Leisingera sp. 204H and P. gallaeciensis ATCC 700781, were able to stimulate the metamorphosis of Hydroides larvae (FIG. 7A-C). These results substantiate the use of the MMK system in marine bacteria during a host-microbe interaction.

To test whether marine bacteria harboring MMK plasmids with fluorescent proteins are amenable to live cell imaging during a host-microbe interaction, we created microcosms containing biofilms of Leisingera sp. 204H bacteria and added competent Hydroides larvae. After incubation of bacteria and larvae for 24 hours, biofilms of Leisingera sp. 204H bacteria were clearly visible when strains expressed gfp or mRuby from respective MMT plasmids and visualized by fluorescent microscopy, while bacteria and the biofilms were difficult to visualize by light microscopy without fluorescently tagged bacteria (FIG. 7C). Many Hydroides larvae had undergone metamorphosis into juvenile animals on the biofilmed surface. Intriguingly, fluorescent Leisingera sp. 204H bacteria could be prominently observed within the gut of Hydroides juveniles (FIG. 7A-C). These results provide a proof-of-concept that the MMK system provides experimental tools for observation and experimentation of marine host-microbe interactions.

Whether and how bacteria and the animal are harmed or benefit from bacteria-stimulated metamorphosis remains a prominent question in the field. Live bacteria within the gut of Hydroides juveniles have never been observed previously. The presence of bacteria within the gut of Hydroides juveniles opens a possibility that Roseobacter bacteria might benefit from stimulating Hydroides to undergo metamorphosis because they can later colonize the tubeworm's digestive tract. Previous work have shown that Hydroides is able to feed on bacteria as the sole food source. Our present observation that Hydroides juveniles ingest marine bacteria substantiates these findings and suggest that Hydroides might undergo metamorphosis in response to some bacteria because they might provide a source of food. The modular platform as provided herein for genetic engineering brings us new abilities to uncover genetic function of marine bacteria and harness marine microbes for applied purposes.

### Materials and Methods

### Bacterial Culture

A list of strains used in this study, their isolation sources, accession numbers, and their minimum inhibitory concentration can be found in Table 1:

| Strain | | | |
|---|---|---|---|
| no. | Strain | Genotype | Source |
| NJS5 | Pseudoalteromonas | StrR | Huang et al., |
| | luteoviolacea HI1 | | 2012 |
| NJS23 | Pseudoalteromonas | StrR, ΔmacB | (Shikuma et |
| | luteoviolacea HI1 | | al., 2014) |
| NJS623 | Pseudoalteromonas | Wild type | (Alker et al., |
| | sp. MMG005 | | 2021) |
| NJS595 | Pseudoalteromonas | Wild type | (Sneed et al., |
| | sp. PS5 | | 2014) |
| NJS597 | Pseudoalteromonas | StrR | This Study |
| | sp. PS5 | | |
| NJS662 | Endozoicomonas | Wild type | DSMZ |
| | montiporae CL-33 | | |
| NJS445 | Vibrio harveyi | Wild type | Stanley |
| | | | Maloy strain |
| NJS675 | Vibrio harveyi | StrR | This Study |
| NJS302 | Shimia sp. | Wild type | This Study |
| NJS204 | Leisingera sp. | Wild type | Cavalcanti |
| | 204H | | et al. 2020 |
| | | | MRA |
| NJS339 | Leisingera sp. | StrR | This Study |
| | 204H | | |
| NJS491 | Leisingera sp. | Wild type | Deogaygay |
| | HS012 | | et al. 2021 |
| | | | MRA |
| NJS678 | Nereida sp. | Wild type | Alker et al. |
| | MMG025 | | in review |
| NJS408 | Phaeobacter | Wild type | (Ruiz-Ponte |
| | gallaeciensis | | et al., 1998) |
| | ATCC 700781 | | |
| NJS412 | Phaeobacter | StrR | This Study |
| | gallaeciensis | | |
| | ATCC 700781 | | |
| pNJS488 | Escherichia coli | TpR SmR recA thi pro | (de Lorenzo |
| | S17-1 | (rK− mK+) RP4: 2-Tc: | and Timmis, |
| | | Mu: Km Tn7 λpir | 1994) |
| NJS604 | Escherichia coli | MG1655 RP4-2- | (Jackson et |
| | MFDpir | Tc::[ΔMu1::aac(3)IV- | al., 2020) |
| | | ΔaphA-Δnic35- | |
| | | ΔMu2::zeo] | |
| | | ΔdapA::(erm-pir) | |
| | | ΔrecA | |

Environmental strains of marine bacteria were isolated and cultured on MARINE BROTH 2216™ (Difco) and seawater tryptone media. The marine bacteria were incubated at 25° C., and cultures were shaken at 200 rpm. Antibiotic selections E. coli SM10pir and S17-1pir were cultured in LB at 37° C., shaking at 200 rpm. E. coli MFDpir (Ferrieres et al. 2010) was cultured in LB supplemented with 0.3 mM Diaminopimelic acid (DAP). For E. coli, antibiotic selections with Ampicillin, Kanamycin, Chloramphenicol were performed using a concentration of 100 μg/mL.

### Plasmid Construction & Assembly

Construction of the transposon backbone was performed by digesting the pLOF-km plasmid with BamHI, amplifying the BsaI and RFP region from pBTK402 and combining the fragments with Gibson Assembly following previously documented protocols. New plasmid parts were made using PCR-amplified fragments and Gibson Assembly. The list of new, BTK and YTK plasmid parts used in this study is available in Table 2:

| | | | | | | | | |
|---|---|---|---|---|---|---|---|---|
| | | 5′ | 3′ | | | | | gene |
| Plasmid | Type | Site | Site | Description | Marker | Origin | Source | no. |

| | | | | | | | |
|---|---|---|---|---|---|---|---|
| pBTK001 | Entry | N/A | N/A | Entry vector | CamR | p15A | (Leonard |
| | vector | | | for generating | | | et al., 2018) |
| | | | | new parts | | | |
| pYTK008 | Connector | 1 | 1 | ConLS′ | CamR | ??? | (Lee et |
| | | | | connector | | | al., 2015) |
| pBTK107 | Promoter | 2 | 2 | CP25 | CamR | ColE1 | (Leonard |
| | | | | promoter, RBS | | | et al., 2018) |
| ??? | Promoter | 2 | 2 | ptac promoter, | CamR | ColE1 | ??? |
| | | | | RBS | | | |
| pBTK205 | Coding | 3 | 3 | GFP optim-1 | CamR | ColE1 | (Leonard |
| | sequence | | | | | | et al., 2018) |
| pYTK034 | Coding | 3 | 3 | mRuby2 | CamR | ??? | (Lee et |
| | sequence | | | | | | al., 2015) |
| pBTK206 | Coding | 3 | 3 | Nanoluc | CamR | ColE1 | (Leonard |
| | sequence | | | | | | et al., 2018) |
| pBTK305 | Terminator | 4 | 4 | T7 terminator | CamR | ColE1 | (Leonard |
| | | | | | | | et al., 2018) |
| pYTK073 | Connector | 5 | 5 | ConRE′ | CamR | ??? | (Lee et |
| | | | | connector | | | al., 2015) |
| pBTK301 | Terminator | 6 | 7 | BBa_B0015 | CamR | ColE1 | (Leonard |
| | | | | Terminator | | | et al., 2018) |
| pBTK402 | Origin, | 8 | 8 | mRFP1 | KanR | RSF1010 | (Leonard |
| | Marker | | | dropout | | | et al., 2018) |
| | Origin, | 8 | 8 | Tn10 | KanR | R6k | |
| | Marker | | | transposon | | | |
| | Origin, | 8 | 8 | Tn7 | GentR | R6k | |
| | Marker | | | transposon | | | |

Fragments were then assembled using GOLDEN GATE ASSEMBLY™ (Thermo Fisher Scientific, Waltham, MA) and either BsaI or BsmBI, depending on the construct. The kanamycin backbone assemblies were electroporated into S17 cells and shuttled to MFD cells for mating. The CRISPRi assemblies were electroporated directly into MFD cells and subsequently mated.

### Biparental Conjugation in Marine Bacteria

Donor strains of E. coli (MFDpir or SM10pir) containing the mobilizable plasmids were grown under antibiotic selection in LB with the appropriate supplements (0.3 mM DAP for E. coli MFDpir). The biparental mating was performed as previously described (Leonard et al. 2018) with modifications for the marine bacteria. Several colonies of the recipient strains were inoculated and grown overnight in liquid culture. Recipient and donor cultures were spun down (4000×g for 2 minutes) in a 1:1 OD ratio. All donor supernatant was removed leaving only the cell pellet. All but 100 μL of the recipient supernatant is removed and the cell pellet is resuspended. The recipient suspension is transferred to the donor pellet, which is resuspended with the recipient cells. Two 50 μL spots are plated onto Marine broth media containing 0.3 mM DAP.

### Violacein Extraction

P. luteoviolacea containing the CRISPRi plasmid targeting the VioA gene was struck onto NSWT media containing 200 μg/mL of Streptomycin and Kanamycin and incubated overnight at 25 C. Single colonies were inoculated into 5 mL of liquid media containing the same antibiotic concentrations. Cultures were incubated at 25 C, shaking at 200 rpm between 18 and 20 hours. Cultures were removed from the incubator and standardized to an OD of 1.5. The cells were then pelleted and the supernatant was removed. The cell pellet was resuspended in 200 μL of 100% ethanol. The resuspended cells were pelleted and the supernatant containing the crude extract was recorded on a Biotek Synergy HT plate reader (Vermont, USA) using the Gen5 program (v2.00.18) with an endpoint reading at 580 nm.

Hydroides elegans Culture

Hydroides elegans adults were collected from Quivira Basin, San Diego, California. The larvae were cultured and reared as previously described (Nedved and Hadfield 2008, Shikuma et al 2014). The larvae were maintained in beakers containing filtered artificial seawater (35 PSU) and were given new beakers with fresh water daily. The larvae were fed living Isochrysis daily. The larvae were used for metamorphosis assays once they reached competency (between 5 and 7 days old).

### Metamorphosis and Colonization Assays

Biofilm metamorphosis assays were performed using previously described methods (Huang and Hadfield 2003, Shikuma et al. 2014, Alker et al. 2020). Bacteria were struck onto Marine Broth plates and incubated overnight at 25 C. Up to 3 single colonies were inoculated into MB broth and incubated overnight (between 15 and 18 hours), shaking at 200 rpm. Cultures were pelleted at 4000 g for 2 minutes, the spent media was removed and the cell pellets were washed twice with filtered ASW. The concentration of the cells was diluted to OD600 of 1 and four 100 μL aliquots of the cell concentrate were added to 96-well plates. The cells were given between 2 and 3 hours to form biofilms, then the planktonic cells were removed and the adhered cells were washed twice with filtered ASW. Between 20 and 40 larvae were added to each well in 100 μL of filtered ASW. Metamorphosis was scored after 24 hours. Four biological replicates were performed on different days using separately spawned batches of larvae.

Colonization assays were performed using the same preparation principles as described above with few modifications. Visualization chambers (Lab-Tek, Cat #155411) were used for setting up the metamorphosis assay, then subsequently imaged. Inductive strains containing constitutively expressed GFP/mRuby/nanoluc plasmids were struck out onto MB media containing 300 μg/mL Kanamycin. Several colonies were inoculated into 5 mL MB media with antibiotics. Cells were washed and allowed to form biofilms as described above. Cell concentrations ranging between OD 0.2 and OD 1 were used to elicit optimal metamorphosis depending on the bacterial species being probed for colonization. Larvae were concentrated and the resident filtered ASW was treated with 300 μg/mL Kanamycin. Larvae were imaged 24 hours later.

This example describes new transposon vectors (designated Tn7 and Tn10) that are compatible with the standardized genetic parts system and can stably integrate into the genome of a marine bacterium such as a Pseudoalteromonas and/or a Roseobacter species.

### Example 5: A Modular Plasmid Toolkit Applied in Marine Proteobacteria Reveals Functional Insights During Bacteria-Stimulated Metamorphosis

This example describes data demonstrating that a modular plasmid toolkit as provided herein, as applied in the marine Proteobacteria, reveals functional insights during bacteria-stimulated metamorphosis.

A conspicuous roadblock to studying marine bacteria for fundamental research and biotechnology is a lack of modular synthetic biology tools for their genetic manipulation. Here, we applied, and generated new parts for, a modular plasmid toolkit to study marine bacteria in the context of symbioses and host-microbe interactions. To demonstrate the utility of this plasmid system, we genetically manipulate the marine bacterium Pseudoalteromonas luteoviolacea, which stimulates the metamorphosis of the model tubeworm, Hydroides elegans. Using these tools, we quantify constitutive and native promoter expression, develop reporter strains that enable the imaging of host-bacteria interactions, and knock down a secondary metabolite and a host-associated gene using CRISPR interference (CRISPRi). We further demonstrate the broader utility of this modular system for rapidly creating and iteratively testing the genetic tractability and modification of marine bacteria that are known to be associated with diverse host-microbe symbioses. These efforts enabled the successful transformation of 12 marine strains across 2 proteobacterial classes, 4 orders and 10 genera. Altogether, the present study demonstrates how synthetic biology strategies enable the investigation of marine microbes and marine host-microbe symbioses with broader implications for environmental restoration and biotechnology.

In this work, we utilize a modular plasmid toolkit, and contribute new marine modification kit (MMK) plasmid parts, to study bacteria-stimulated metamorphosis in the Gammaproteobacterium, P. luteoviolacea. We also demonstrate the broader utility of this plasmid system by manipulating marine Alphaproteobacteria and Gammaproteobacteria that have been shown previously to be involved in diverse host-microbe symbioses.

### Results

Toolkit-Enabled Quantitative Promoter Expression in P. luteoviolacea

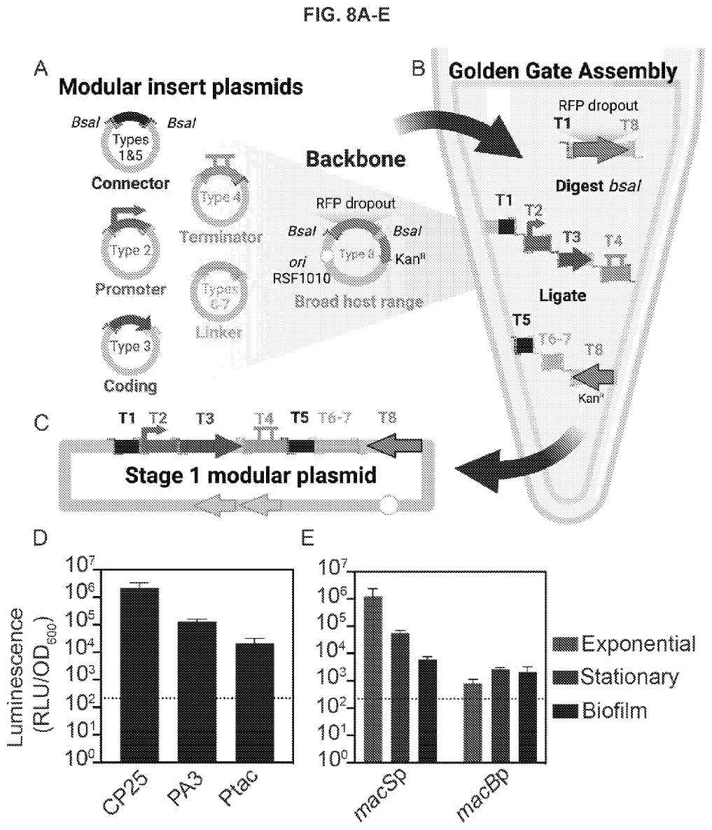

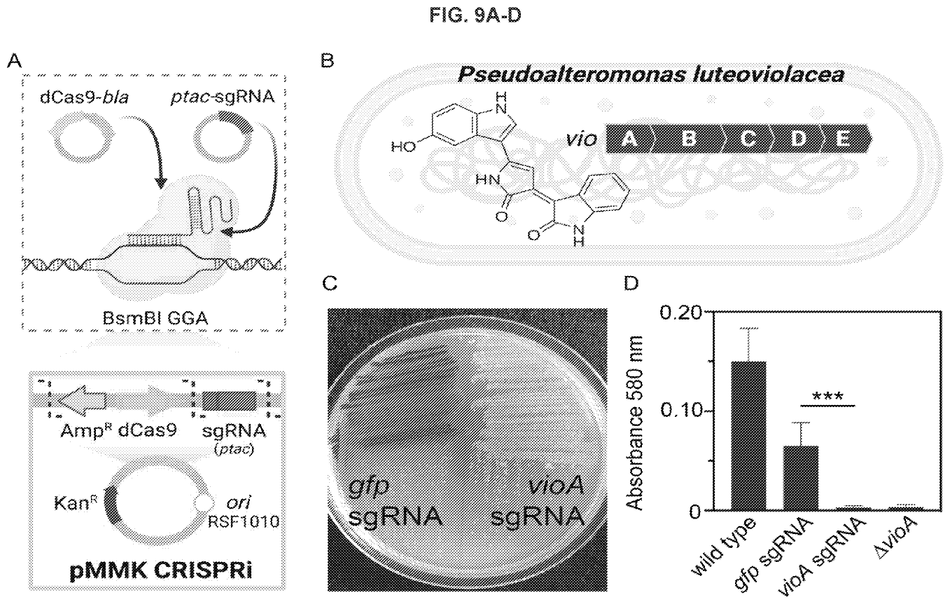

To test the application of modular genetic tools in marine bacteria, we identified a set of preexisting parts from the YEAST TOOLKIT™ and BEE TOOLKIT™ (BEE MICROBIOME TOOLKIT™ (BTK)) platforms (17, 18) and use GOLDEN GATE ASSEMBLY™ (Thermo Fisher Scientific, Waltham, MA) (14) for the rapid and modular construction of plasmids (FIG. 8A-C). Each type of part is denoted by the functional role it performs and the directional 4 bp overlaps generated by the flanking Type IIS (BsaI) restriction sites. The modular parts include: Type-1 and Type-5 stage-2 connectors with BsmBI recognition sites (17, 18), a Type-2 promoter with ribosome binding site (RBS), a Type-3 protein coding sequence (CDS), a Type-4 terminator, an optional Type-6 repressor and Type-7 promoter and a Type-8 backbone. For this work, we selected a broad-host-range plasmid backbone containing a kanamycin resistance gene, a reporter coding sequence (fluorescent gfp-optim1, mRuby or NanoLuciferase [Nluc]), T7 terminator and a stage 2 assembly connector. The backbone has an RSF1010 origin of replication, known to replicate in a broad range of gram positive and negative bacterial hosts (43). A promiscuous origin of transfer and plasmid-encoded conjugative machinery (44) enabled domestication-free conjugative transfer with MFDpir auxotrophic host E. coli cells (45).

FIG. 8A-E illustrates a schematic overview of the modular plasmid system and quantitative promoter measurements. (A) Modular GGA plasmid parts with flanking BsaI cut sites (dashed lines). Overlapping 4 bp overhangs are color coordinated. The modular broad host range backbone (pBTK402) contains inverted BsaI cut sites and an RFP dropout. (B) GOLDEN GATE ASSEMBLY™ (Thermo Fisher Scientific, Waltham, MA) is performed in a one-tube reaction by digesting the backbone and insert part plasmids with BsaI and ligating with T4 ligase. (C) A modular stage 1 plasmid is complete when all overlapping inserts are successfully assembled in order. (D) Biofilm luciferase assay of P. luteoviolacea strains expressing replicative plasmids with different constitutive promoters driving a Nluc gene (CP25-Nluc-T7, PA3-Nluc-T7, Ptac-Nluc-T7). Luminescence, as relative luminescence units (RLU), is normalized to optical density at 600 nm (OD₆₀₀) and plotted on a log base 10 scale. The dashed line indicates the detection limit three standard deviations above the P. luteoviolacea (no plasmid) control (Y=214 RLU/OD₆₀₀). Plotted is the mean of three biological replicates+/−standard deviation for all experiments. (E) Luciferase assay comparing native MACs promoters macS and macB attached to a Nluc coding sequence across different modes of growth. N=3 biological replicates. The dashed line indicates the detection limit three standard deviations above the P. luteoviolacea (no plasmid) control (Y=218 RLU/OD600).

To apply the modular genetic tools to a marine symbiosis model, we explored constitutive and native promoter expression in P. luteoviolacea. We assembled plasmids with one of five promoters fused to Nluc and conjugated the plasmids into P. luteoviolacea. We utilized two existing constitutive promoters, PA3 and CP25, previously shown to work in diverse bee gut microbes (17)(46)(47). We designed a Ptac LacO constitutive promoter part (pMMK201), which is a hybrid of the lac and trp promoters amplified from the pANT4 plasmid (48). We also constructed two native P. luteoviolacea promoters driving the expression of the MACs structural genes; promoters from the MACs sheath (macS promoter, pMMK203) and baseplate (macB promoter, pMMK202) genes. We observed at least 10-fold more luminescence signal compared to background in all constitutive promoters tested (FIG. 8D, dotted line), with the CP25 promoter exhibiting a 10,000 fold increase in luminescence in the biofilm phase. P. luteoviolacea macSN luc luminescence was elevated 1,000-fold in exponential growth as compared to 100-fold in stationary and 10-fold in biofilm phase, when compared to the detection limit (FIG. 8E). In contrast, the macB, baseplate promoter exhibited similar levels of luminescence among each phase, approximately 10-fold higher than the detection limit (FIG. 8E).

Functional CRISPRi Knockdown of Secondary Metabolite Biosynthesis in P. luteoviolacea

While previous studies in P. luteoviolacea have used gene knockouts to interrogate gene function, these approaches are time consuming and low-throughput. We therefore tested whether P. luteoviolacea is amenable to gene knockdown via CRISPR interference (CRISPRi) (FIGS. 9A and 9B) (49, 50). As a proof-of-concept, we targeted the vioA gene that encodes a key enzyme in the biosynthesis of violacein (51), which gives P. luteoviolacea its characteristic purple pigment (FIG. 9B). To facilitate assembly for and expression in P. luteoviolacea, we modified the BsmBI cut site in the dCas9 part plasmid to include the bla ampicillin resistance gene, resulting in the part plasmid (pMMK601). We replaced the existing PA1 promoter with Ptac in the guide RNA part plasmid targeting GFP (pMMK602). An assembled plasmid containing dCas9 and a small guide RNA (sgRNA) targeting the non-template strand of vioA (pMMK603) was conjugated into P. luteoviolacea resulting in the visible absence of the purple pigment associated with violacein production on the plate (FIG. 9C). A plasmid with a sgRNA targeting the non-template strand of gfp was included as a control. A significant reduction of violacein production was observed between cultures of P. luteoviolacea strains expressing the vioA and gfp targeting CRISPRi plasmids (p=0.0007, FIG. 9D). The lack of violacein in the vioA knockdown strain was comparable to that of a P. luteoviolacea strain with an in-frame deletion of vioA (FIG. 9D). These results demonstrate the successful implementation of CRISPRi for gene knockdown in P. luteoviolacea.

FIG. 9A-D illustrates data showing: CRISPRi knockdown of secondary metabolite production in P. luteoviolacea. (A) Modular CRISPRi parts were adapted from a previous study to include dCas9-bla and Ptac-sgRNA parts, pMMK601 and pMMK602, respectively. Part plasmids are combined and a GOLDEN GATE ASSEMBLY™ (Thermo Fisher Scientific, Waltham, MA) is performed with BsmBI. (B) The CRISPRi system was assembled with an sgRNA targeting the vioA gene (pMMK603) and employed to knock down violacein production in P. luteoviolacea. (C) Knockdown of vioA resulted in a visible difference in purple pigment produced in bacteria grown on agar plates. (D) Extraction of overnight cultures revealed a significant reduction in violacein production (measured at 580 nm) between wildtype containing a non-targeting gfp plasmid and knockdown strains (***p=0.0007, Dunnett's T3 multiple comparisons test). Columns show the mean (N=8) and error bars indicate standard deviations.

Functional CRISPRi Knockdown and Visualization of P. luteoviolacea During a Tubeworm-Microbe Interaction.

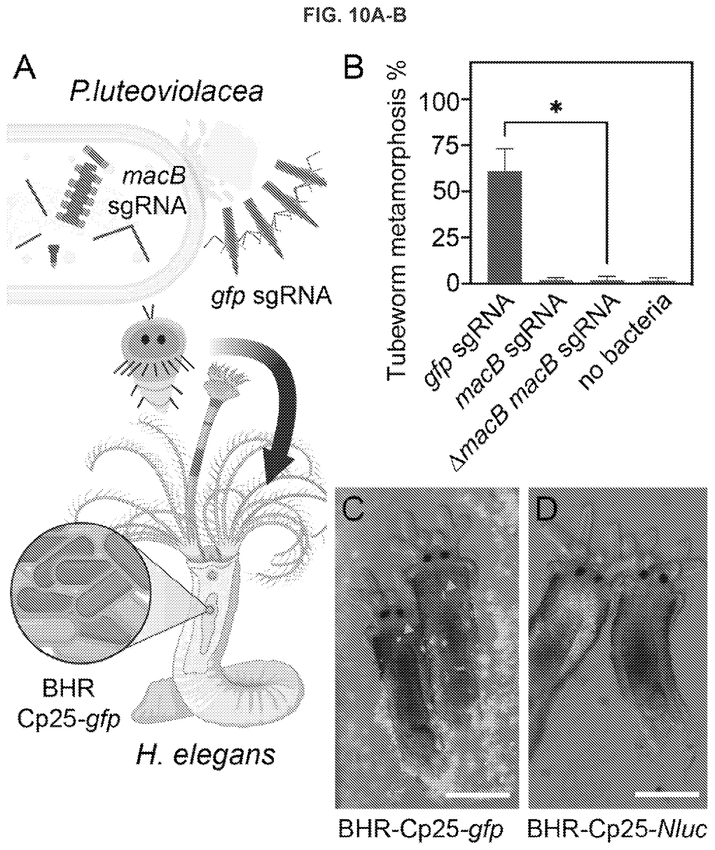

We next tested whether CRISPRi would be functional in the context of a marine symbiotic host-microbe interaction by targeting the macB gene, which encodes the MACs baseplate, an essential component of the MACs complex that induces tubeworm metamorphosis (39, 40) (FIG. 10A). Biofilm metamorphosis assays were performed comparing P. luteoviolacea strains with sgRNAs targeting macB (pMMK604) or a sgRNA targeting gfp as a control (FIG. 10B). The strain containing the macB sgRNA exhibited significantly reduced levels of tubeworm metamorphosis compared to the gfp-sgRNA control (FIG. 10B; Mann Whitney test, p=0.029). The reduction of metamorphosis stimulation in the macB sgRNA knockdown strain was comparable to that of a P. luteoviolacea strain with an in-frame deletion of macB carrying the gfp-sgRNA control plasmid (FIG. 10B). These results demonstrate that CRISPRi paired with a modular plasmid framework is a streamlined tool for interrogating gene function in vivo during a marine host-microbe interactions.

FIG. 10A-D illustrates data showing functional knockdown of MACs and visualization of P. luteoviolacea during the tubeworm-microbe interaction. (A) Schematic depicting P. luteoviolacea and the production of MACs, which induce tubeworm metamorphosis. CRISPRi guide RNA targeting the macB MACs baseplate gene, rendering it unable to induce metamorphosis. A strong fluorescent reporter strain (BHR-CP25-gfp) enabled live tubeworm-bacteria visualization. (B) Single strain biofilm metamorphosis assay with CRISPRi modular plasmid targeting gene, macB induced significantly less metamorphosis in H. elegans (P=0.029, Mann Whitney test). P. luteoviolacea containing a plasmid with a sgRNA targeting gfp or a P. luteoviolacea ΔmacB strain were used as controls. Biofilm concentrations were made with cells at OD₆₀₀ 0.2. Plotted is the average of 3 biological replicates (N=3) performed on separate occasions. Averages were calculated with four technical replicates with each well containing 20-40 worms. Error bars show standard deviation. (C and D) Fluorescence micrographs of juvenile Hydroides elegans imaged 24 hours after the competent larvae were exposed to inductive biofilms of P. luteoviolacea containing constitutively expressed (C) CP25-gfp (D) CP25-Nluc. Nluc expressing plasmids were used as the negative control to exclude autofluorescence previously documented in the tubeworms (21). Yellow arrows show accumulation of fluorescent bacteria in the intestinal tract. Scale bar is 100 μm.

To date, bacteria have not been visualized during or after the stimulation of metamorphosis in Hydroides. To test whether marine bacteria harboring a toolkit plasmid are amenable to live cell imaging when in association with juvenile tubeworms, we created microcosms with biofilms of P. luteoviolacea containing plasmids encoding CP25-gfp-T7 (gfp) or CP25-Nanoluc-T7 (Nluc) and added competent Hydroides larvae. After the microcosms were incubated for 24 hours, biofilms of gfp-tagged P. luteoviolacea were clearly observed when visualized by fluorescence microscopy (FIG. 10C, D). P. luteoviolacea stimulated metamorphosis while carrying a modular and replicative plasmid, and intriguingly, fluorescent bacteria were observed being ingested by the Hydroides juveniles (FIG. 10C). Bacteria can be seen collecting (see yellow arrows in the pharynx), then moving in a peristaltic fashion toward the gut. In contrast, the bacteria and their biofilms were difficult to visualize by light microscopy without fluorescent bacteria (FIG. 10D), consistent with previous observations of Hydroides larvae (21). Taken together, the modular plasmid system enables the optimization of live imaging and experimentation during a marine host-microbe interaction.

### Genetic Manipulation of Diverse Marine Proteobacteria.

Given the success of genetic manipulation of P. luteoviolacea, we tested whether other, more diverse marine Proteobacteria are amenable to genetic manipulation via the modular genetic toolkit technology. To this end, we isolated or acquired representative bacteria that are known to play critical roles in symbioses with marine plants or animals in the ocean (FIG. 11A; Table S1). To enable genetic selection using antibiotics, we determined the minimum inhibitory concentration for each bacterial strain tested against kanamycin (Table S1). When conjugation was performed using the broad-host-range (RSF1010) plasmid backbone, CP25 promoter, gfp reporter and T7 terminator, we observed fluorescent expression in 12 marine strains across 2 proteobacterial classes, 4 orders and 10 genera. Eight of the strains were made tractable for the first time, including bacteria from Pseudoalteromonas, Endozoicomonas, Cobetia, Shimia, Nereida, Leisingera, and Phaeobacter genera (FIG. 11B). Adaptations to the conjugation protocol and use of constitutive promoters driving gfp enabled visual confirmation of successful conjugation (FIG. 11B).

FIG. 11A-B illustrates data showing that diverse marine Proteobacteria are amenable to plasmid uptake and stable replication of toolkit plasmids. (A) Maximum likelihood whole genome phylogeny of 12 strains selected for manipulation and successfully transformed in this study (52, 53). All strains used in this study are known for their interaction with a range of marine biota and the icons depicting their host association are shown in the vertical box. Gammaproteobacteria strains are highlighted in purple and Alphaproteobacteria strains are shown in gold. Scale bar is 0.4 and bootstraps were generated using the rapid-bootstrapping method (54). The tree was rooted at the midpoint with FIGTREE v 1.4.4™, a graphical viewer of phylogenetic trees, (B) Fluorescence microscopy of overnight cultures containing constitutively expressed RSF1010 on fluorescence vector (CP25-gfp-T7). Scale bar is 5 μm.

TABLE S1
List of strains used in this study. NT = antibiotic sensitivity not tested:

| Strain | | | | | MIC Kan | |
|---|---|---|---|---|---|---|
| no. | Strain | Genotype | Class | Order | (μg/mL) | Source |
| NJS005 | Pseudoalteromonas | StrR | Gamma | Alteromonadales | 200 | (39) |
| | luteoviolacea HI1 | | | | | |
| NJS023 | Pseudoalteromonas | StrR, | Gamma | Alteromonadales | NT | (40) |
| | luteoviolacea HI1 | ΔmacB | | | | |
| NJS595 | Pseudoalteromonas | Wild type | Gamma | Alteromonadales | NT | (36) |
| | sp. PS5 | | | | | |
| NJS597 | Pseudoalteromonas | StrR | Gamma | Alteromonadales | 200 | This |
| | sp. PS5 | | | | | study |
| NJS445 | Vibrio harveyi | Wild type | Gamma | Vibrionales | NT | Stanley |
| | | | | | | Maloy |
| NJS675 | Vibrio harveyi | StrR | Gamma | Vibrionales | 200 | This |
| | | | | | | study |
| MT002 | Photobacterium | Wild type | Gamma | Vibrionales | 400 | Alison |
| | mandapamensis | | | | | Gould |
| | svers.3.2 | | | | | |
| NJS662 | Endozoicomonas | Wild type | Gamma | Oceanospirillales | 100 | (111) |
| | montiporae CL-33 | | | | | |
| NJS775 | Cobetia sp. MMG | Wild type | Alpha | Oceanospirillales | 200 | This |
| | | | | | | study |
| NJS302 | Shimia sp. MMG | Wild type | Alpha | Rhodobacterales | 300 | This |
| | | | | | | study |
| NJS409 | Ruegeria pomeroyi | Wild type | Alpha | Rhodobacterales | 300 | (88) |
| | DSS-3 | | | | | |
| NJS491 | Phaeobacter sp. | Wild type | Alpha | Rhodobacterales | 300 | (89) |
| | HS012 | | | | | |
| NJS408 | Phaeobacter | Wild type | Alpha | Rhodobacterales | 300 | (87) |
| | gallaeciensis | | | | | |
| | ATCC 700781 | | | | | |
| | (DSM 26640) | | | | | |
| NJS412 | Phaeobacter | StrR | Alpha | Rhodobacterales | 300 | This |
| | gallaeciensis | | | | | Study |
| | ATCC 700781 | | | | | |
| | (DSM 26640) | | | | | |
| NJS204 | Leisingera sp. 204H | Wild type | Alpha | Rhodobacterales | 200 | (90) |
| NJS339 | Leisingera sp. 204H | StrR | Alpha | Rhodobacterales | 200 | This |
| | | | | | | Study |
| NJS678 | Nereida sp. MMG025 | Wild type | Alpha | Rhodobacterales | 200 | (95) |

| | | | |
|---|---|---|---|
| pNJS488 | Escherichia coli | TpR SmR recA thi pro (rK− mK+) | |
| | S17-1 | RP4: 2-Tc: Mu: Km Tn7 λpir | (112) |
| NJS604 | Escherichia coli | MG1655 RP4-2-Tc::[ΔMu1::aac(3)IV-ΔaphA- | |
| | MFDpir | Δnic35-ΔMu2::zeo]ΔdapA::(erm-pir) ΔrecA | (45) |
| pNJS033 | Escherichia coli | thi thr leu tonA lacY supE | |
| | SM10 | recA::RP4-2-Tc::Mu Km λpir | (113) |

TABLE S2
List of plasmids used in this study.

| | | 5′ | 3′ | | | | |
|---|---|---|---|---|---|---|---|
| Plasmid | Type | Site | Site | Description | Marker | Origin | Source |
| pBTK001 | Entry | N/A | N/A | Entry vector for | CamR | p15A | (17) |
| | vector | | | generating new parts | | | |
| pYTK008 | Connector | 1 | 1 | ConLS′ connector | CamR | ColE1 | (18) |
| pBTK107 | Promoter | 2 | 2 | CP25 promoter, RBS | CamR | ColE1 | (17) |
| pBTK121 | Promoter | 2 | 2 | PA3 promoter, RBS | CamR | p15A | (17) |
| pMMK201 | Promoter | 2 | 2 | ptac promoter, RBS | CamR | ColE1 | This study |
| pMMK202 | Promoter | 2 | 2 | HI1 macB promoter, | CamR | ColE1 | This study |
| | | | | RBS | | | |
| pMMK203 | Promoter | 2 | 2 | HI1 macsS promoter, | CamR | ColE1 | This study |
| | | | | RBS | | | |
| pYTK047 | GFP | 2 | 4 | gfp dropout (internal | CamR | ColE1 | (18) |
| | Dropout | | | BsaI sites) | | | |
| pBTK205 | Coding | 3 | 3 | gfp optim-1 | CamR | ColE1 | (17) |
| | sequence | | | | | | |
| pYTK034 | Coding | 3 | 3 | mRuby2 | CamR | ColE1 | (18) |
| | sequence | | | | | | |
| pBTK206 | Coding | 3 | 3 | Nanoluc | CamR | ColE1 | (17) |
| | sequence | | | | | | |
| pBTK305 | Terminator | 4 | 4 | T7 terminator | CamR | ColE1 | (17) |
| pYTK073 | Connector | 5 | 5 | ConRE′ connector | CamR | ColE1 | (18) |
| pBTK402 | Origin, | 8 | 8 | mRFP1 dropout | KanR | RSF1010 | (17) |
| | Marker | | | | | | |
| pBTK527 | Origin, | ConLS′ | ConRE′ | BsmBI sites flanking | KanR | RSF1010 | (17) |
| | Marker | | | spacer | | | |
| pBTK614 | dCas9 | ConL1 | ConRE | dead cas9 | AmpR | ColE1 | (17) |
| pMMK601 | dCas9-bla | ConL1 | ConRE | dead cas9 with amp | AmpR | ColE1 | This study |
| | | | | resistance gene | | | |
| pBTK615 | sgRNA | ConLS | ConR1 | sgRNA targeting gfp | AmpR | ColE1 | (17) |
| | ptac | | | sgRNA targeting gfp | | | |
| pMMK602 | sgRNA | ConLS | ConR1 | driven by ptac | AmpR | ColE1 | This study |
| | VioA | | | sgRNA targeting PL | | | |
| pMMK603 | sgRNA | ConLS | ConR1 | vioA driven by ptac | AmpR | ColE1 | This study |
| | macB | | | sgRNA targeting PL | | | |
| pMMK604 | sgRNA | ConLS | ConR1 | macB driven by ptac | AmpR | ColE1 | This study |
| pMMK809 | Stage 1 | 1 | 5 | pBTK402-PA3- | KanR | RSF1010 | (17) |
| | assembly | | | Nluc-T7 | | | |
| pMMK810 | Stage 1 | 1 | 5 | pBTK402-CP25- | KanR | RSF1010 | (17) |
| | assembly | | | Nluc-T7 | | | |
| pMMK811 | Stage 1 | 1 | 5 | pBTK402-Ptac- | KanR | RSF1010 | This study |
| | assembly | | | Nluc-T7 | | | |
| pMMK812 | Stage 1 | 1 | 5 | pBTK402-macBp- | KanR | RSF1010 | This study |
| | assembly | | | Nluc-T7 | | | |
| pMMK813 | Stage 1 | 1 | 5 | pBTK402-macSp- | KanR | RSF1010 | This study |
| | assembly | | | Nluc-T7 | | | |
| pMMK814 | Stage 1 | 1 | 5 | pBTK402-CP25-gfp- | KanR | RSF1010 | (17) |
| | assembly | | | T7 | | | |
| pMMK815 | Stage 1 | N/A | N/A | pCRISPRi-dCas9- | KanR/AmpR | RSF1010 | This study |
| | assembly | | | bla-Ptac-gfp | | | |
| pMMK816 | Stage 1 | N/A | N/A | pCRISPRi-dCas9- | KanR/AmpR | RSF1010 | This study |
| | assembly | | | bla-Ptac-vioA | | | |
| pMMK817 | Stage 1 | N/A | N/A | pCRISPRi-dCas9- | KanR/AmpR | RSF1010 | This study |
| | assembly | | | bla-Ptac-macB | | | |

### Discussion

New Genetic Tools Provide Insights about Bacteria-Stimulated Metamorphosis.

We tested a modular plasmid toolkit on a tractable marine bacterium, P. luteoviolacea, that promotes the metamorphosis of the tubeworm Hydroides elegans (40, 41, 55) and produces a range of bioactive secondary metabolites (26, 29, 56, 57). We expand the tools available for functional interrogation of bacteria-stimulated metamorphosis in P. luteoviolacea by quantifying gene expression by luminescence assay (FIGS. 8D and E), and using CRISPRi to knock down the secondary metabolite, violacein (FIGS. 9C and D), and a metamorphosis-associated gene, macB (FIG. 10B) during the bacteria-tubeworm interaction. Distinct patterns of sheath (macSp) (41, 58) and baseplate (macBp) promoter induction suggest distinct mechanisms of gene regulation within the MACs gene cluster. Expression of the sheath gene appears dynamic, i.e. sensitive to bacterial mode of growth, while baseplate gene expression appears static and less sensitive to mode of growth. Although MACs are known to produce two effectors that stimulate tubeworm metamorphosis and kill eukaryotic cells (41, 58), the environmental conditions that promote MACs production remain poorly characterized. The tools developed here could help to characterize the conditions under which P. luteoviolacea MACs are produced and assembled and could help in the development of MACs or other Contractile Injection Systems for use in biotechnology as molecular syringes (59, 60). The modular tools in this work open new capabilities for interrogating bacterial biology, including the ability to quantify gene expression, knock down gene expression for rapid functional testing and visualize bacteria during an interaction.

Whether and how bacteria and the animal are harmed or benefit from the interaction during bacteria-stimulated metamorphosis remains a prominent question in the field (38, 61, 62). Previous work by Gosselin et al. have shown that Hydroides is able to feed on bacteria as the sole food source (63). But until the present work, live bacteria within the gut of Hydroides juveniles had not been observed (FIG. 0C). The visualization of transgenic bacteria in Hydroides will enable future lines of research that can help us dissect the role of microbiome seeding in bacteria-stimulated metamorphosis. More broadly, our results showcase the feasibility of using a modular plasmid toolkit to test hypotheses about bacteria-stimulated metamorphosis, and provides a framework for the interrogation of other bacteria and their products that promote host-microbe symbioses (36, 64, 65).

Toolkit Compatibility in Diverse Proteobacteria and their Potential for Future Study

In this work, we explore genetic tractability and gene function in 12 ecologically relevant marine Proteobacteria. These strains belong to two Proteobacterial classes, half of which were transformed for the first time (FIG. 11). Compatibility with the broad-host-range plasmid backbone (RSF1010 origin of replication) suggests that all strains may be amenable to further manipulation with other toolkit coding sequences including luminescence reporters, complementation and CRISPRi gene knockdown.

The Gammaproteobacteria strains transformed in this study are a diverse selection of symbiosis-associated strains representing 5 genera (FIG. 11A). To our knowledge, this is the first report of genetic tractability in strains from the genera, Endozoicomonas and Cobetia (FIG. 11B). Endozoicomonas species are among the most abundant bacterial symbionts in some corals and other marine hosts but are notoriously difficult to culture, therefore limiting our understanding of their functional roles in animal holobionts (66-68). Related strains of Cobetia, have been implicated in thermotolerance against bleaching in coral experiments with probiotic consortium treatments (69). The transformation of the representative Endozoicomonas and Cobetia strains in this study is a considerable step towards exploring function in coral host-microbiome interactions at a critical time to encourage the responsible stewardship of coral probiotic interventions (6, 70, 71). The genetic transformation of Pseudoalteromonas sp. PS5 in this study presents an opportunity to explore secondary metabolite production, including the coral metamorphosis-inducing compound, tetrabromopyrrole (FIG. 11) (36). Other Gammaproteobacteria successfully transformed in this study include two bioluminescent strains, Vibrio harveyi and Photobacterium mandapamensis svers.3.2, which are associated with luminescence and organogenesis in squid (72, 73) and fish (74), respectively, but also as pathogens in aquaculture (75, 76) and corals (77, 78) (FIG. 11A). In summary, the development of methods and established tractability of several new strains and genera have significant implications for the future of bacterial genetic development in established and emerging symbiosis systems.

The Alphaproteobacteria strains tested in this study fall within the Roseobacter group (FIG. 11A), an ecologically important group of bacteria known to play a role in sulfur and carbon cycling on marine phytoplankton (79-81), in part due to their special capacity for lateral gene transfer and biofilm formation (82, 83). Related host-derived strains have been explored as probiotics for the aquaculture industry due to their antibacterial capacity (84-86). We explored the compatibility of the toolkit with the tractable, phytoplankton-associated species of Phaeobacter gallaeciensis (87), and Ruegeria pomeroyi (88), and demonstrate compatibility for the first time with invertebrate microbiome-associated strains Phaeobacter sp. HS012 (89) and Leisingera sp. 204H (90) (FIG. 11). The tractability of bacteria within the genus Shimia sp. has not previously been explored prior to this study, which may be of interest for coral microbiome studies in simulating impacted environments (91-94). To this end, we tested and transformed a Shimia strain from a coral disease outbreak isolated in Curaçao. Furthermore, there are no previous reports of genetics in Nereida sp. MMG025 or other species in the Nereida genus, which have been isolated from kelp (95) and are associated with gall formations (96, 97). Tractability in this strain could help guide further understanding of microbe-seaweed interactions (98, 99), kelp aquaculture and the development of kelp probiotics (100). Taken together, the framework used in this study to establish tractability can be used in future studies to explore the function of marine Roseobacter species in a wide range of symbiosis systems from the environment.

### Conclusion

The exemplary modular plasmid toolkit described here provides a basis for streamlining the genetic manipulation of marine bacteria for basic and applied purposes. These tools open up new possibilities to studying marine microbes in the context of plant and animal interactions, or with challenging and diverse non-model bacteria, ultimately helping us harness marine microbes for research, bioproduction and biotechnology.

### Methods

### Bacterial Culture

A list of strains used in this study, their isolation sources, accession numbers, and their minimum inhibitory concentration can be found in Table S1. Environmental strains of marine bacteria were isolated and cultured on MARINE BROTH 2216™ (BD Difco) and or natural seawater tryptone (NSWT) media (1 L 0.2 filtered natural seawater, 2.5 g Tryptone, 1.5 g Yeast, 1.5 mL glycerol). MB and NSWT media are used interchangeably throughout the study; however, experiments were always conducted using only one media type. Marine bacteria were incubated between 25-30° C., and cultures were shaken at 200 rpm. All liquid cultures were inoculated with a single colony and incubated between 16-18 hours, unless otherwise indicated. E. coli SM10pir and S17-1pir were cultured in LB at 37° C., shaking at 200 rpm. E. coli MFDpir (45) was cultured in LB supplemented with 0.3 mM Diaminopimelic acid (DAP). For E. coli, antibiotic selections with Ampicillin, Kanamycin, Chloramphenicol were performed using a concentration of 100 μg/mL.

### Plasmid Construction & Assembly

GOLDEN GATE ASSEMBLY™ (Thermo Fisher Scientific, Waltham, MA)-compatible parts from the BTK, YTK and MMK (17, 18) can be found in Table S2. New plasmid parts were made by PCR amplifying insert and backbone fragments and combining them with Gibson Assembly with a 2:1 ratio (insert: backbone) (101). PCR amplification was performed with custom primers (Table S3), a high-fidelity DNA polymerase (Primestar, TaKaRa) and purified using a DNA CLEAN AND CONCENTRATOR™ kit (Zymo Research). Part plasmids were assembled to make a stage 1 plasmid using GOLDEN GATE ASSEMBLY™ (Thermo Fisher Scientific, Waltham, MA), using T4 DNA ligase (Promega) and either BsaI or BsmBI, depending on the construct. Single-tube assembly was performed by running the following thermocycler program (BsaI/BsmBI): 37/42° C. for 5 minutes, 16° C. for 5 minutes, repeat 30×, 37/55° C. for 10 minutes, 80° C. for 10 minutes. The assemblies were directly electroporated into S17-1pir cells, confirmed by colony PCR (ECONOTAQ PLUS GREEN™, LGC Biosearch) with internal primers and then shuttled to MFDpir cells for conjugation. The Ptac-sgRNA part plasmid with guide RNA was created to ensure expression of the sgRNA in P. luteoviolacea. To increase plasmid assembly efficacy, a BsmBI recognition site was moved to include the bla ampicillin resistance gene withing the dCas9 part, enabling dual selection for positively assembled clones with kanamycin and ampicillin. The CRISPRi assemblies were electroporated directly into SM10pir cells and shuttled to MFDpir cells for conjugation.

TABLE S3
List of Primers used in this study.

| Primer | Sequence |
|---|---|
| p107_bbamp_F | TATGTGAGACCAGACCAATAAAAA |
| p107_bbamp_R | CGTTTGAGACCGACTACGGTTA |
| macb_seq_f | ATGAGCCGAGAATTATCCTTGAG |
| sheath_seq_f | CATGGCGTCATAGCAGTACA |
| ptac_gbsn_F2 | TAACCGTAGTCGGTCTCAAACGGCACTCCCGTTCT |
| | GGATAAT |
| ptac_gbsn_R2 | TTTTTATTGGTCTGGTCTCACATAGGGACAACTCC |
| | AGTGAAAAG |
| pBTK615_ptac_macB1_ | TCGGCTCGTATAATGTGTGGAAGCTCGGGGATCTG |
| sgRNA_F | TCGTG |
| pBTK615_ptac_macB1_ | TTTTAACTTGCTATTTCTAGCTCTAAAACCACGAC |
| sgRNA_R | AGATCCCCGAGCTT |
| pBTK107_macB_ | GATAACCGTAGTCGGTCTCAAACGGAAGTTTCTGC |
| promoter_gbsn_F1 | GGTGCTTTT |
| pBTK107_macB_ | TTTTTATTGGTCTGGTCTCACATAAGATTACCTTAT |
| promoter_gbsn_R1 | TAATGTTATTAATGAGCAT |
| pBTK107_sheath_ | GATAACCGTAGTCGGTCTCAAACGACACCGACTTT |
| promoter_gbsn F1 | ACCCTATCTCG |
| pBTK107_sheath_ | TTTTTATTGGTCTGGTCTCACATAGTTTTTCCTTAC |
| promoter_gbsn_R1 | GTTGATAATTACATTC |
| pBTK107_CP25_F | TGAGGGGGCTGGTATAATCA |
| gRNA_VioA5_F | CACATATTTATGTTCATAAACTCGAAG |
| pBTK615_ptac_seqF1 | ACAGACACTGCGACAACGTG |
| pBTK615_gRNA_GFP | CGTCTAATTCCACGAGGATTG |
| p615_ptac_gRNA_ | TCGGCTCGTATAATGTGTGGTTTATGTTCATAAAC |
| VioA5_F2 | TCGAA |
| p615_ptac_gRNA_ | TTTTAACTTGCTATTTCTAGCTCTAAAACTTCGAGT |
| VioA5_R2 | TTATGAACATAAA |
| p615_ptac_vector_ | CCACACATTATACGAGCCGA |
| amplification_R | |
| p615_vector_ | GTTTTAGAGCTAGAAATAGCAAGTTAAAA |
| amplification_F | |
| p615-ptac_F2 | CAATTAATCATCGGCTCGTATAATGTGTGGCGTCT |
| | AATTCCACGAGGATTG |
| p615-ptac_R2 | TACGAGCCGATGATTAATTGTCAACAGCTCTTCAG |
| | TGAGACGGTATTGCG |
| 61A2_Kan_intF2 | CTGCCTCGGTGAGTTTTCTC |
| | CTTTTCTACGGGGTCTGACGCGTCTCATGCTCCTC |
| 61C9_p614-bla_R1 | AGTGGAACGAAAACTCACG |
| | GTGAACACTCTCCCGGCTGAAATCTGCTCGTCAGT |
| 61D1_p614-bla_F1 | GGTG |
| 61D2_p614-bsmBI_R1 | GACGCGTCTCATGCTCCT |
| 61D2_p614-bsmBI_F1 | CTCCCGGCTGAAATCTGC |
| 62B5_pBTK107_seq_F | TGGATAACCGTAGTCGGTCTC |
| 62B6_pBTK107_seq_R | GGATTTGTTCAGAACGCTCGGTT |

### Biparental Conjugation in Marine Bacteria

E. coli donor strains (MFDpir or SM10pir) containing the mobilizable plasmids were grown under antibiotic selection in LB with the appropriate supplements (including 0.3 mM DAP for E. coli MFDpir). Conjugations were performed as previously described (17) with modifications for culturing marine bacteria. Briefly, several colonies of the recipient strains were inoculated and grown overnight in liquid culture. Recipient and donor cultures were spun down (4000×g for 2 minutes) in a 1:1 OD₆₀₀ ratio. All donor supernatant was removed leaving only the cell pellet. All but 100 μL of the recipient supernatant is removed and the cell pellet is resuspended. The recipient suspension is transferred to the donor pellet, which is resuspended with the recipient cells. Two 50 μL spots are plated onto marine media (supplemented with 0.3 mM DAP for MFD-mediated conjugations). Spots are resuspended in 500 μL of liquid marine media and 100 μL is plated onto marine media containing antibiotic selection, according to the minimum inhibitory concentration (Table S1) Several of the bacteria take longer to grow or do not reach a high optical density (i.e. Endozoicomonas, Reugeria, Nereida) in culture. Slower-growing marine bacteria were conjugated by growing larger initial volumes of culture and spinning down the entire culture to reach 1:1 donor: host ratios.

### Phylogeny

Strains or close representative strains used in this study were compiled into a genome group on PATRIC v3.6.12 (102). A whole genome phylogenetic codon tree composed of 100 single copy genes (103) was performed using the Phylogenetic Tree Service (104-106). A Maximum likelihood phylogeny was generated using the best protein model found by RaxMLv8.2.11 (107), which was LG. Bootstraps were generated using the rapid bootstrapping algorithm (54). The tree was visualized with FIGTREE v1.4.4.™ and was rooted at the mid-line.

### Luciferase Culture and Assay

P. luteoviolacea containing plasmids with constitutive or native promoters driving NanoLuciferase (Nluc) were inoculated into 5 mL of MB or NSWT media with appropriate antibiotics and grown at 25° C. at 200 rpm for 24 hours. Each biological replicate was represented by a separate culture. Cultures used for the growth phase assay were inoculated as a 1:100 dilution with the appropriate antibiotic, and then incubated at 25° C. and shaking at 200 rpm. The luminescence of cultures were measured at exponential (OD₆₀₀ of 0.35-1.0), early stationary (OD₆₀₀ 1.0-1.45) or late stationary (OD₆₀₀ 2.38-2.54) phases. For biofilm cultures, 1.5 mL of stationary-phase culture was pelleted and plated as a single spot on NSWT or MB plates. Biofilm plates were incubated at 20-25° C. for 24-28 hours. Each spot was scraped with a pipette tip and resuspended in 200 μL of NSWT or MB media before being resuspended in NSWT or MB. Luciferase reactions were performed with 100 μL of bacterial culture or biofilm resuspension aliquoted into opaque white walled 96-well plates (Corning #3642), with a modified protocol as written for NANO-GLO LIVE CELL ASSAY SYSTEM™ (Promega cat #N2011). Briefly, bacteria and the final reagent mix were read at a 1:1 ratio. Luminescence was measured on a microplate FILTERMAX F5™ (Molecular Devices) reader with a custom program on the SOFTMAX PRO 7™ software. Readings were done on the kinetic luminescence mode at 2-minute intervals for 20 minutes in total, using a 400 ms integration time, a 1 mm height read, and no other optimization or shaking settings. The detection limit is defined as three standard deviations above nine biological and technical replicates of WT P. luteoviolacea. Raw data were normalized to the OD₆₀₀ of the culture used and plotted with an N=3 biological replicates.

### Violacein Extraction

The specified P. luteoviolacea strains were struck onto NSWT media containing 200 μg/mL of streptomycin and kanamycin and incubated overnight at 25° C. Single colonies were inoculated into 5 mL of liquid media containing the same antibiotic concentrations. Cultures were incubated at 25° C., shaking at 200 rpm between 18 and 20 hours. Cultures were removed from the incubator and standardized to an OD₆₀₀ of 1.5. The cells were pelleted and the supernatant was removed. The cell pellet was resuspended in 200 μL of 100% ethanol. The resuspended cells were pelleted and the supernatant containing the crude extract was recorded on a Biotek Synergy HT plate reader (Vermont, USA) using the Gen5 program (v2.00.18) with an endpoint reading at 580 nm.

### Microscopy

Microscopy was performed using AXIO OBSERVER.Z1™ (Zeiss) inverted microscope equipped with an AXIOCAM 506™ mono camera and NEOFLUAR10X/0.3 PH1™/DICI™ (Hydroides co-cultures) or APOCHROMAT 100x™/1.4 Oil DICIII™ (bacteria only) objectives. The HE EGFP FILTER SET 38™ (Zeiss) was used to capture GFPoptim-1 expression and HE MRFP FILTER SET 63™ (Zeiss) was used to capture mRuby2 expression. For nanoluciferase controls, images were captured using the same fluorescence exposure times as the GFPoptim-1 and mRuby2 labeled strains of the same species.

Bacterial culture (2 l) were added to freshly prepared 1% saltwater low-melt agarose (Apex catalog #20-103, Bioresearch products) pads on glass slides and coverslips were placed on top. Hydroides elegans with bacteria were imaged in the visualization chambers (chambered coverglasses, Lab-Tek catalog #155411PK) they were prepared in.

Hydroides elegans Culture

Hydroides elegans adults were collected from Quivira Basin, San Diego, California. The larvae were cultured and reared as previously described (40, 108). Larvae were maintained in beakers containing filtered artificial seawater (35 PSU) and were given new beakers with water changes daily. The larvae were fed live Isochrysis and cultures were maintained as described previously. The larvae were used for metamorphosis assays once they reached competency (between 5 and 7 days old) (109).

Hydroides elegans Metamorphosis Assays

Biofilm metamorphosis assays were performed using previously described methods (39, 40, 110). Briefly, bacteria were struck onto Marine Broth plates with 300 μg/mL kanamycin as appropriate and were incubated overnight at 25° C. Up to 3 single colonies were inoculated into liquid broth and incubated overnight (between 15 and 18 hours), shaking at 200 rpm. Cultures were pelleted at 4000 g for 2 minutes, the spent media was removed and the cell pellets were washed twice with filtered artificial sea water (ASW). The concentration of the cells was diluted to OD₆₀₀ of 1 and four 100 μL aliquots of the cell concentrate were added to 96-well plates. The cells were given between 2 and 3 hours to form biofilms, then the planktonic cells were removed and the adhered cells were washed twice with filtered ASW. Between 20 and 40 larvae were added to each well in 100 μL of filtered ASW. Metamorphosis was scored after 24 hours. Four biological replicates were performed on different days using separately spawned batches of larvae.

Chambered metamorphosis assays were performed using the same preparation principles as described above with few modifications. Visualization chambers (Lab-Tek, Cat #155411) were used for setting up the metamorphosis assay, then subsequently imaged. Inductive strains containing constitutively expressed GFP/mRuby/nanoluc plasmids were struck out onto MB media containing 300 μg/mL kanamycin. Several colonies were inoculated into 5 mL MB media with antibiotics. Cells were washed and allowed to form biofilms as described above. Cell concentrations ranging between OD₆₀₀ 0.2 and 1 were used to elicit optimal metamorphosis depending on the bacterial species being probed for colonization. Larvae were concentrated and the resident filtered ASW was treated with 300 μg/mL kanamycin. Larvae were imaged 24 hours later.

### Minimum Inhibitory Concentration Protocol

### Day 1

1. Streak out marine microbes onto Marine Agar plates and incubate overnight at 25° C.-28° C. Some strains take longer to grow to single colonies. Incubation times longer than 24 hours may be required for some slower growing strains. Monitor growth in future steps accordingly.

### Day 2

2. Inoculate a single colony into 5 mL Marine Broth (2216) media in the late afternoon/early evening. Incubate overnight at 25 C, shaking at 200 rpm.

### Day 3

3. Measure optical density (OD600) of overnight culture growth and document.
4. Pipette 100 μL of overnight culture from each strain onto Marine Agar plates containing either Strep (for background resistance), Kan (MIC), Gent (MIC) at each concentration (25 μg/mL, 50 μg/mL, 100 μg/mL and 200 μg/mL).
5. Use beads or plate spreader to spread the culture evenly on the plate. Remove the beads and incubate overnight at 25° C. overnight.

### Day 4

6. Observe and document growth on all concentration plates. For the MICs (Kan/Gent) identify the lowest concentration of media in which no colonies are observed. If there are no colonies at any concentration, screen lower concentrations of Kanamycin/Gentamicin in the media (i.e. 5, 10, 15, 20 μg/mL).
7. For the Streptomycin Resistant strain, select a single colony from the highest dose of Streptomycin that has growth and streak it out onto a plate containing the same concentration and a plate containing the next highest concentration of antibiotics. Incubate overnight at 25 C.

### Day 5

8. Observe and document growth on both the MIC plates (2 days old) and the Streptomycin resistance plates. If Streptomycin resistance is sustained at less than 200 μg/mL, then continue step 7, passaging the strains onto higher concentrations of antibiotics. Once robust growth occurs at 200 μg/mL, inoculate a single colony into 5 mL Marine Broth containing Strep 200 μg/mL and incubate 25° C. overnight, shaking at 200 rpm.

### Day 6 or Later

Store culture in cryovials for long term storage. Add 500 μL overnight culture to 500 μL 50% glycerol, pipette mix and store in the −80° C. for future use.

### NanoLuciferase Assay

### Day 1: Streak Out Strains

1. Streak out all strains from frozen stock onto the appropriate antibiotic plates. Including a positive and negative control. Pseudoalteromonas luteoviolacea expressing kanamycin resistant backbone was struck onto NSWT with 300 μg/mL of Kanamycin. Pseudoalteromonas sp. PS5 expressing kanamycin resistant backbone was struck onto Marine Broth (MB) with 300 μg/mL of Kanamycin. Wild Type marine bacteria were grown on MB only media.

### Day 2: Create Sub-Culture

2. Create sub-culture by inoculating 5 colonies from the plate into a 5 mL tube with appropriate media and antibiotics. Repeat for each strain into a separate 5 mL tube. Biological replicates should be in separate sub-cultures.
3. Inoculate and let grow for 24 hours. Next step will need to start 24 hours from when you do this step.

### Spot Cultures for Biofilm

4. Take the Optical Density (OD 600 nm) measurement of the sub-cultures to ensure they are in stationary phase before spotting them onto the agar plate.
5. Take 1.5 ml of sub-culture and transfer into centrifuge tube. Spin down to concentrate bacteria. Spin cultures at 5000 RCF for 10 minutes. Remove supernatant and discard.
6. Resuspend bacterial pellet with 75 μL of the appropriate liquid media.
7. Spot the entire 75 μL onto a single agar plate of the appropriate media and antibiotics.
Inoculate plate for 24 hours.

### Day 3: Innoculate Experimental Cultures

8. Inoculate experimental culture from the sub-culture created in Day 2 using a 1:100 dilution of media to subculture.
9. In 125 mL flask add 25 mL of media and 250 μL of sub-culture. Repeat for each strain and biological replicate into its own flask.
10. Take optical density measurement right after inoculation to have a time point 0 (TO).
11. Continue to take optical density measurements to determine the major growth phases that will be tested in the NanoLuciferase assay. Test 2-3 strains to determine where the OD is at. May need to retest every 30 minutes in the beginning to capture exponential phase. To measure OD: Fill cuvettes in with 1 mL of sample and flick each vial before taking OD.

### Measurement.

### Day 3: Performing NanoLuciferase Assay

12. Prepare a luciferase assay with a solution consisting of 2.5 μl of Buffer, 0.5 μl of substrate and 17.5 μl of water (luciferase assay solution called “mastermix”) for blanks reactions blanks

$$2.5 µL of Buffer \times 4 = 10 µL of Buffer + 0.5 µL of Substrate$$

$$17.5 µL of water \times 4 = 70 µL$$

20 μL of blank media per well
Run the plate with the blanks making sure to label to results as blanks in the computer software. Always have the machine read the whole plate and not just selected cells that have samples.
13. Prepare luciferase plate by loading blank samples first to see if any of the wells including the blank are showing signal before the start of the assay.
14. Prepare the sample only plate in a regular 96-well PCR plate 100 μL of each sample culture laid out the same as the luciferase plate.
Keep the layout the same so you can use a multichannel pipette to transfer the samples from the PCR plate to the luciferase plate.
Shake or mix each flask before pipetting out the sample so that it is evenly mixed
15. Dilute the samples when making the samples plate.
Past assays have been successful with the following dilution scheme.
Pseudoalteromonas luteoviolacea was diluted in a range of 1:10 to 1:100.
Pseudoalteromonas sp. PS5 was diluted in a range of 1:1,000 to 1:100,000.
Late stationary and biofilm growth phases showed higher expression and required larger dilutions. Additionally, constitutive promoters required larger dilutions than native promoters.
Use serial dilution of 1:100 in a regular 96-well plate first then proceed to do 10-fold dilutions to get to the above dilution levels.
16. Prepare the luciferase experimental plate. Use the mastermix reaction template to calculate the amount for your number of samples. i.e. For 72 reactions prep 80 reactions for mastermix 80×2.5 μL Buffer/Substrate=200 μL Buffer+10 μL Substrate

$$80 \times 17.5 μL MilliQ Water = 1400 MQ$$

20 μL of mastermix per well
17. Pipette 20 μL of mastermix in each well first.
18. Then, pipette 20 μL of sample into luciferase plate with mastermix using a multichannel pipette. Needs to be done as quickly as possible ˜1 minute is ideal.
19. At each time point that you run the assay measure the optical density to normalize your luciferase data by OD. Fill cuvettes in with 1 mL of sample.
Flick each vial to mix before taking OD measurement.

### Marine Bacteria Plasmid Conjugation

This protocol can be used to mate broad host range plasmids (including pBTK and other plasmids containing RSF1010 origins of replication) into diverse marine bacteria
Media: Plates to streak out Plasmids contained in E. coli mating strains (SM10, S-17, MFD): LB plates containing 100 μg/mL of the appropriate antibiotic(s) (plus 0.3 mM DAP for MFD cells). Liquid media for incoluations: LB, Natural Sea Water Tryptone (NSWT) and/or Marine Broth (Difco 2216) media.
Plates to streak out the marine bacteria: Natural SWT and/or Marine Agar plates
Plates to perform the mating: Natural SWT and/or Marine Agar plates—containing 0.3 mM DAP if the plasmids are in MFD cells.
Plates for selection: Natural SWT and/or Marine Agar plates containing 200 μg/mL Kanamycin
Storing stocks: 50% glycerol
Antibiotic Stocks: 100 mg/mL filter sterilized antibiotic stocks for the appropriate selectable markers
Media Supplements: 2,6 Diaminopimelic Acid (DAP) filter sterilized at 30 mM working stock
Dry materials:
   Sterile sticks
   Beads
   1.5 mL microcentrifuge tubes
   Petri dishes
Confirmation materials
   GoTaq for colony PCR
   Primers to confirm plasmid in colony PCR
For Fluorescent plasmids: Microscope with Fluorescence filters for visual confirmation of Colonies.
1. Streak out the marine bacteria and E. coli strains containing the plasmids to be mated using the streak plate method. Be sure to check the library to determine the location of the strains and which media, antibiotics, and/or media supplements (e.g., Diaminopimelic Acid (DAP)) should be used for each strain/plasmid.
2. Incubate the plates overnight in a plastic bag in the incubator.
E. coli strains are incubated at 37° C.
Marine bacteria strains are incubated at 25° C.

### Day 2

3. [Morning] Inoculate 3 colonies of the marine strains into 5 mL NSWT or Marine Broth. Incubate at 25° C., shaking at 200 rpm. Some marine bacteria may take longer to grow than others, which could slightly shift the timeline of this protocol. If the marine bacteria is a slower grower, you can opt to inoculate the marine bacteria in the morning of day two and the plasmids at night for day two, and spot mate on the morning of Day 3. This will increase the protocol to 7 days total.
4. [Morning] Inoculate a single colony of plasmid into 5 mL LB broth+100 μg/mL of appropriate antibiotic(s) (i.e., 5 μL of 100 mg/mL stock) and/or media supplements (i.e. 0.3 mM DAP=50 μL of 30 mM stock). Incubate at 37° C. shaking at 200 rpm.
5. [Evening] Marine bacteria: Remove 1 mL of culture per each plasmid being mated and put into a 1.5 mL microcentrifuge tube. Include an additional 1 mL of culture as a negative control (i.e. If you are mating 1 marine bacterium with 3 different plasmids; GFP/mRuby/Nanoluc, you will need (4) 1 mL aliquots of culture).
6. [Evening] Plasmid mating strains: Remove 1 mL of culture for each marine bacterium being mated and put into a 1.5 mL microcentrifuge tube. Include an additional 1 mL of culture as a negative control (i.e. If you are mating 1 marine bacteria with 3 different plasmids, GFP/mRuby/Nanoluc, you will need (2) 1 mL aliquots of each plasmid).
7. Centrifuge all culture aliquots 4000×g for 00:02:00
8. Remove all of the supernatant of the plasmid aliquots.
Remove all but 100 μL of the supernatant for the marine bacteria aliquots.
9. Resuspend the marine bacteria in the remaining 100 μL of supernatant. Put the marine bacteria negative control to the side for now.
10. Pipet up the 100 μL of resuspended cells and transfer it to a tube containing the plasmid cell pellet. Pipet mix to homogenize the plasmid cells and marine bacteria cells together. Repeat process for all different plasmids+marine bacteria being mated.
11. For the Plasmid negative controls, add 100 μL of marine bacteria media to the plasmid cell pellet and resuspend.
12. After all strains are resuspended and/or mixed, plate (2) 50 μL spots onto mating plates. Marine bacteria negative control plate. The media plates used for mating should be determined by the type of E. coli cells used for mating.
SM10 and S17 cells can be mated on regular NSWT/Marine Broth MFD cells should be mated on NSWT/Marine Broth containing 0.3 mM DAP.
Plasmid negative control plate(s).
1 experimental plate for each marine bacteria+plasmid combo.
13. Incubate all plates at 25° C. overnight with their lids facing up.

### Day 3

14. Aliquot 500 μL of NSWT or Marine Broth into a 1.5 mL microcentrifuge tube for each spot.
One spot can be plated for each negative control.
Both spots should be mated for each experimental mating plate.
15. Pick up the spot with a pipette tip and place into the microcentrifuge tube containing the media. Resuspend the bacteria in the media. Shake the pipette tip vigorously then remove the tip and pipette mix or vortex to homogenize the bacteria in the media.
16. Plate 100 μL of the cells onto selection plates; NSWT or Marine agar plates containing antibiotics at the appropriate concentration determined by the marine bacteria's MIC. Spread with beads. Incubate overnight at 25° C.

### Day 4

17. Select 3-6 colonies per spot to patch plate and perform a Colony PCR. Patch onto a new selection plate. From here forward, the strains must be grown on media containing the appropriate concentration of antibiotics to retain the plasmid.
18. Run a gel to confirm colony PCR
19. Streak out positive clones onto NSWT or Marine Agar selection plates and incubate at 25° C. overnight. Be sure to include at least one clone from each spot mating to create 2 copies for storage.

### Day 5

20. Inoculate a single colony of each strain into 5 mL NSWT or Marine Broth media containing antibiotics. Incubate overnight at 25° C. shaking at 200 rpm.

### Day 6

21. Make a glycerol stock of the new strains (2 copies/strain, 2 strains/mating) and store them in the strain library.

### Violacein Quantification

Crude Violacein Extraction Protocol for the Quantification of Violacein Production after CRISPRi Knockdown in P. luteoviolacea.

### Day 1

1. Streak out bacteria on Natural Seawater Tryptone (NSWT) plates. Strains requiring antibiotic selective pressure are struck out on NSWT plates containing 200 μg/ml of Streptomycin and Kanamycin. Incubate plates overnight at 25° C.

### Day 2

2. Inoculate single colonies into 5 mL NSWT (containing antibiotics if appropriate) and incubate at 25° C., shaking 200 rpm for 20-24 hours.

### Day 3

3 Remove the cultures from the incubator. Measure the culture density with a spectrophotometer at OD600. Dilute the cultures so that each strain is equivalent to an OD of 1.5 in 1 mL of cells. (x mL)(Measured OD600)=(1.5 OD)(1 mL).
4. Centrifuge the cells at 4000×g for two hours.
5. Remove the supernatant (approximately 1 mL) while avoiding the cell pellet.
6. Resuspend the cell pellet in 200 μL of 100% Ethanol P212121 Catalog #BE-BDH1156
7. Centrifuge the crude extract at 4000×g for two hours.
8. Transfer the supernatant (approximately 200 μL) to a 96-well plate in replicate. Be sure to include a 100% Ethanol blank in replicate as a control.
9. Measure the absorbance on a plate reader (SYNERGY HT™ micro plate reader, Biotek) at measuring an endpoint reading at 580 nm.
The software GEN5 VERSION 2.00.18™ was used to perform measurement.
10. Start the run and use the Blank 580 values for data. Export and plot.

### Golden Gate Assembly™ (Thermo Fisher Scientific, Waltham, MA) Protocol

### Day 1

1. Streak out the plasmid parts that will be used in the assembly onto LB agar plates with the appropriate antibiotics (Chloramphenicol 100 μg/mL for Type 1-7 parts; Kanamycin or Gentamicin 100 μg/mL for Type 8 backbone parts). Incubate overnight at 37 C.

### Day 2

2. Inoculate a single colony into 25 mL of LB plus antibiotics in the late afternoon/early evening. Incubate overnight at 37 C while shaking at 200 rpm.

### Day 3

3. Spin the culture at 5000 g for 20 minutes. Remove the supernatant, resuspend the pellet in 1 mL water. Perform a plasmid miniprep (ZYPPY™ miniprep or Omega E.Z.N.A. PLASMID MINI KIT II™) following the standard kit protocols.
4. Measure the Plasmid DNA concentration on a spectrophotometer.
5. Perform the GOLDEN GATE ASSEMBLY™ (Thermo Fisher Scientific, Waltham, MA):
Dilute backbone plasmid parts and add 10 fmol to the reaction
Dilute the insert plasmid parts and add to 20 fmol of each insert to the reaction
   Add 2 μL T4 Ligase Buffer (Promega)
   Add 1 μL T4 Ligase
   Add 1 μL BsaI or BsmBI endonuclease
   Add X water up to a 20 μL reaction
Run the thermocycler program for BsaI/BsmBI as follows:

| | | |
|---|---|---|
| A | B | C |
| Step | Temperature | Time |

| 1 | 37/42 | C. | 5 | minutes |
|---|---|---|---|---|
| 2 | 16 | C. | 5 | minutes |
| Cycles 1-2 | Repeat 30× | | | |
| 3 | 37 C./55 C. | | 10 | minutes |
| 4 | 80 | C. | 10 | minutes |

### Day 4

7. Dilute 2× or electroporate 2 μL of the GGA directly into electrocompetent cells (i.e. SM10, S17). Recover 1+ hours and plate on LB Agar media containing the correct antibiotic concentrations. Incubate at 37 C overnight.

### Day 5

8. Screen colonies for correct insert and perform colony PCR with primers spanning assembly junctions.

### Day 6

9. Streak out clones that yield a band onto LB Agar media containing the correct antibiotic concentrations and incubate at 37 C overnight.
10. Inoculate a single colony into 25 mL LB broth containing the correct antibiotic concentrations and incubate at 37 C overnight.

### Day 7

11. Store overnight culture in cryovials for long term storage. Add 500 μL of culture to 500 μL of 50% glycerol and store in −80 C freezer.
12. With remaining culture, Centrifuge at 5000 g for 20 minutes. Remove the supernatant, resuspend the pellet in 1 mL water. Perform a plasmid miniprep (ZYPPY™ miniprep or Omega E.Z.N.A. PLASMID MINI KIT II™) following the standard kit protocol. Elute with water.
13. Measure the Plasmid DNA concentration on a spectrophotometer.
14. Send the miniprepped plasmid for Sanger or Oxford Nanopore long-read plasmid sequencing to confirm the construct. Store the remaining miniprep for downstream applications (i.e. shuttle into different electrocompetent cell for conjugation).
   (Sigma) sgRNA Cloning Design for Non-Template CDS Targeting

You can design sgRNAs to target 3 regions effectively:
1. Transcription factor binding site/operator binding sites—Targets NT or T
2. The promoter or RNAP binding site—Target NT or T
3. Coding Region—Target NT strand ONLY
1. Open your gene of interest in MACVECTOR™. Identify PAM complement 5′(CCN).
2. Label the PAM site 5′(NGG)3′ on the (template) reverse strand
3. Identify the base pairing region on the Nontemplate (forward) strand. This should be an approximately 20 bp coding region immediate downstream of the PAM complement (CCN-N1 . . . N20).
4. Label the base pairing region on the (template) reverse strand.
5. Highlight the base pairing region and copy the sequence. **Note: MACVECTOR™ only copies in the 5′-3′ direction on the forward (nontemplate) strand***
6. Put the sequence into the reverse complement program to get the NT sgRNA sequence (same sequence as Step 4). Copy the reverse complement sgRNA sequence.
7. Open the pBTK615-ptac plasmid in MACVECTOR™. Feel free to use the original vector (targeting GFP) or another one already made (i.e. pBTK615ptac-VioA5). Highlight the 20 bp sgRNA feature. Go to the editor and delete the sgRNA sequence. Paste the sequence generated in step 6 in its place. ***Note: Do not include the PAM sequence in the new vector file***
8 Double check the plasmid map. Confirm that the base-pairing region sequence is on the forward strand of the pBTK615ptac vector.

### FastCloning Primer Design

9. On the pBTK615-ptac vector containing your sgRNA base pairing sequence, identify a 20-25 bp primer binding region on the plasmid directly upstream of the sgRNA on the forward strand.
10. Use Primer3 to estimate the melting temperature of the vector overlap primer and determine the vector overlap length.
11. Copy the 5′ vector overlap region and sgRNA sequences (˜40-50 bp) to create the forward primer. Label in MACVECTOR™. Paste the forward primer into the Primer Library as pBTK_615_genename#_F
12. Do almost the same thing for the reverse strand. Select the primer and the vector overlap region directly upstream (remember it's 5′ on the reverse strand). Copy the approximately 40 bp region and paste it into the reverse complement program. Copy the output and paste this in the Shikuma Lab Primer Library as pBTK_615_genename#_R.
13. Create confirmation primers that cover the entire base-pairing region. It is okay to include a couple of nucleotides on either end of the vector if necessary to generate an acceptable in Primer3. Make a forward or reverse confirmation primer that can be paired with another primer on the pBTK615-ptac plasmid.
14. order primers

### FastCloning

15. Resuspend and dilute Primers from IDT
16. Set up a 3-step Primestar PCR reaction to amplify the backbone vector (i.e. 615 vectoramp F & 615 vectoramp R). Use 10 ng miniprepped plasmid as the template.
17. Set up a 2-step Primestar PCR reaction to amplify the insert. Do not include a template. Your primers should be complementary to each other over the 20 base pairing region. The purpose of this PCR reaction is to anneal the two primers together.
18. Run a gel using 5 μL of PCR product to confirm insert and backbone vector amplification. The resulting bands should be approximately 70 bp total and may be dispersed on your gel. That is okay.
19. Perform a DNA CLEAN & CONCENTRATOR™ (Zymo) PCR cleanup on your backbone vector and sgRNA inserts. Low yield for the inserts should be expected due to the small fragment size. Confirm success with quality measurements (260/280).
20. Perform a fragment Gibson ASSEMBLY CLONING KIT™ (New England Biolabs) to calculate the necessary vector (100 ng) and insert masses. Ligate in the thermocycler for one hour at 50° C. A 3:1 ratio has been used successfully in the construction of these plasmids.
21. After ligation, dilute the Gibson 4× (add 30 uL to the 10 uL reaction)
22. Electroporate 2 uL into SM10, S17, or MFD Electrocompetent cells. Recover at 200 rpm, 37° C. for one hour. 200 rpm, 37° C., 01:00:00
23. Plate 100 μL of regular and concentrated cells onto spread plates. Incubate overnight at 37° C. C
24. Perform a colony PCR and use the confirmation primers to identify the correct clones (i.e. sgRNA_VioA5F & blaoutF)
25. Streak out positive clones onto LBamp (or other appropriate selective media). Incubate overnight at 37° C.
26. Inoculate a single colony into 25 mL LB plus antibiotics. Incubate overnight at 200 rpm, 37° C.
27. Spin down 4000×g 10 min, overnight cultures and resuspend in 1 mL H2O.
28. Perform a plasmid miniprep (can use ZYPPY PLASMID MINIPREP KIT™ Quantify.
29. Send out sequencing (pBTK sequencing primers).
30. Store confirmed plasmids in the Plasmid library
31. Proceed to CRISPRi GOLDEN GATE ASSEMBLY™ (Thermo Fisher Scientific, Waltham, MA) protocol.

### REFERENCES EXAMPLE 1

1. Krueger F. 2015. Trim Galore: a wrapper tool around Cutadapt and FastQC to consistently apply quality and adapter trimming to FastQ files.
2. Wick R R, Judd L M, Gorrie C L, Holt K E. 2017. Unicycler: Resolving bacterial genome assemblies from short and long sequencing reads. PLoS Comput Biol 13:e1005595.
3. Davis J J, et al. 2020. The PATRIC Bioinformatics Resource Center: Expanding data and analysis capabilities. Nucleic Acids Res 48:D606-D612.
4. Tatusova T, et al. 2016. NCBI prokaryotic genome annotation pipeline. Nucleic Acids Res 44:6614-6624.
5. Arahal D R, et al. 2016. Draft genomic sequence of Nereida ignava CECT 5292T, a marine bacterium of the family Rhodobacteraceae. Stand Genomic Sci 11:1-8.
6. Pujalte M J, et al. 2005. Nereida ignava gen. nov., sp. nov., a novel aerobic marine α-proteobacterium that is closely related to uncultured Prionitis (alga) gall symbionts. Int J Syst Evol Microbiol 55:631-636.
7. Wagner-Dobler I, Biebl H. 2006. Environmental biology of the marine Roseobacter lineage. Annu Rev Microbiol. Annual Reviews.
8. Ondov B D, et al. 2016. Mash: Fast genome and metagenome distance estimation using MinHash. Genome Biol 17.
9. Yoon S H, et al. 2017. Introducing EzBioCloud: A taxonomically united database of 16S rRNA gene sequences and whole-genome assemblies. Int J Syst Evol Microbiol 67:1613-1617.
10. Thompson C C, et al. 2013. Microbial genomic taxonomy. BMC Genomics 14.
11. Kim Y O, et al. 2016. Octadecabacter ascidiaceicola sp. nov., isolated from a sea squirt (Halocynthia roretzi). Int J Syst Evol Microbiol 66:296-301.
12. Morrow K M, et al. 2018. A member of the Roseobacter clade, Octadecabacter sp., is the dominant symbiont in the brittle star Amphipholis squamata. FEMS Microbiol Ecol 94:30.
13. Raina J B, et al. 2010. Do the organic sulfur compounds DMSP and DMS drive coral microbial associations? Trends Microbiol 18:101-108.
14. Howard E C, et al. 2008. Abundant and diverse bacteria involved in DMSP degradation in marine surface waters. Environ Microbiol 10:2397-2410.
15. Sonnenschein E C, et al. 2021. The Roseobacter-Group Bacterium Phaeobacter as a Safe Probiotic Solution for Aquaculture. Appl Environ Microbiol 87:1-15.
16. Damjanovic K, Blackall L L, Webster N S, van Oppen M J H. 2017. The contribution of microbial biotechnology to mitigating coral reef degradation. Microb Biotechnol 10:1236-1243.
17. Egan S, Harder T, Burke C, Steinberg P, Kjelleberg S, Thomas T. 2013. FEMS Microbiol Rev. Oxford Academic.
18. Peixoto R S, et al. 2017. Beneficial microorganisms for corals (BMC): Proposed mechanisms for coral health and resilience. Front Microbiol. Frontiers Media S.A.
19. Stamatakis A. 2014. RAxML version 8: a tool for phylogenetic analysis and post-analysis of large phylogenies. Bioinformatics 30:1312-1313.
20. Wattam A R, et al. 2017. Improvements to PATRIC, the all-bacterial bioinformatics database and analysis resource center. Nucleic Acids Res 45:D535-D542.
21. Cock P J A, et al. 2009. Biopython: freely available Python tools for computational molecular biology and bioinformatics. Bioinformatics 25:1422-1423.
22. Davis J J, et al. 2016. PATtyFams: Protein families for the microbial genomes in the PATRIC database. Front Microbiol 7:118.
23. Edgar R C. 2004. MUSCLE: Multiple sequence alignment with high accuracy and high throughput. Nucleic Acids Res 32:1792-1797.
24. Stamatakis A, et al. 2008. A Rapid Bootstrap Algorithm for the RAxML Web Servers. Syst Biol 57:758-771.
25. Letunic I, Bork P. 2016. Interactive tree of life (iTOL) v3: an online tool for the display and annotation of phylogenetic and other trees. Nucleic Acids Res 44:W242-W245.

### REFERENCES EXAMPLE 2

1. Eddy T D, et al. Global decline in capacity of coral reefs to provide ecosystem services. One Earth 2021; 4: 1278-1285.
2. Hughes T P, et al. Global warming impairs stock-recruitment dynamics of corals. Nature 2019; 568: 387-390.
3. Rohwer F, et al. Diversity and distribution of coral-associated bacteria. Mar Ecol Prog Ser 2002; 243: 1-10.
4. Thompson J R, Rivera H E, Closek C J, Medina M. Microbes in the coral holobiont: Partners through evolution, development, and ecological interactions. Front Cell Infect Microbiol 2014; 4: 176.
5. Bourne D G, Morrow K M, Webster N S. Insights into the Coral Microbiome: Underpinning the Health and Resilience of Reef Ecosystems. http://dx.doi.org/101146 annurev-micro-102215-095440 2016; 70: 317-340.
6. Reshef L, et al. The Coral Probiotic Hypothesis. Environ Microbiol 2006; 8: 2068-2073.
7. Anthony K, et al. New interventions are needed to save coral reefs. Nat Ecol Evol 2017; 1: 1420-1422.
8. van Oppen M J H, et al. Shifting paradigms in restoration of the world's coral reefs. Glob Chang Biol 2017; 23: 3437-3448.
9. van Oppen M J H, Blackall L L. Coral microbiome dynamics, functions and design in a changing world. Nat Rev Microbiol 2019 179 2019; 17: 557-567.
10. Peixoto R S, et al. Beneficial microorganisms for corals (BMC): Proposed mechanisms for coral health and resilience. Front Microbiol 2017; 8: 341.
11. Peixoto R S, et al. Annual Review of Animal Biosciences Coral Probiotics: Premise, Promise, Prospects. 2021.
12. Deutsch J M, et al. Metabolomics Approaches to Dereplicate Natural Products from Coral-Derived Bioactive Bacteria. J Nat Prod 2022; acs.jnatprod.lc01110.
13. Santoro E P, et al. Coral microbiome manipulation elicits metabolic and genetic restructuring to mitigate heat stress and evade mortality. Sci Adv 2021; 7: 3088-3101.
14. Damjanovic K, et al. Experimental inoculation of coral recruits with marine bacteria indicates scope for microbiome manipulation in Acropora tenuis and Platygyra daedalea. Front Microbiol 2019; 10: 1702.
15. Will Probiotics Save Corals or Harm Them?—Scientific American. https://www.scientificamerican.com/article/will-probiotics-save-corals-or-harm-them/. Accessed 11 Feb. 2022.
16. Damjanovic K, et al. The contribution of microbial biotechnology to mitigating coral reef degradation. Microb Biotechnol 2017; 10: 1236-1243.
17. Joseph Pollock F, et al. Coral larvae for restoration and research: A large-scale method for rearing Acropora millepora larvae, inducing settlement, and establishing symbiosis. PeerJ 2017; 2017: e3732.
18. Chamberland V F, et al. New Seeding Approach Reduces Costs and Time to Outplant Sexually Propagated Corals for Reef Restoration. Sci Rep 2017; 7: 18076.
19. Tran C, Hadfield M G. Larvae of Pocillopora damicornis (Anthozoa) settle and metamorphose in response to surface-biofilm bacteria. Mar Ecol Prog Ser 2011; 433: 85-96.
20. Negri A P, et al. Metamorphosis of broadcast spawning corals in response to bacteria isolated from crustose algae. 2001; 223: 121-131.
21. Cavalcanti G S, et al. The Influence of Bacteria on Animal Metamorphosis. Annu Rev Microbiol 2020; 74: 137-158.
22. Tebben J, et al. PLoS One 2011; 6: e19082.
23. Sneed J M, et al. Proc R Soc B Biol Sci 2014; 281.
24. Shikuma N J, et al. Marine Tubeworm Metamorphosis Induced by Arrays of Bacterial Phage Tail-Like Structures. Science (80-) 2014; 343: 529-533.
25. Tebben J, et al. Chemical mediation of coral larval settlement by crustose coralline algae. Sci Rep 2015; 5.
26. Agarwal V, et al. Biosynthesis of polybrominated aromatic organic compounds by marine bacteria. Nat Chem Biol 2014; 10: 640-647.
27. Gamal A, et al. Biosynthesis of coral settlement cue tetrabromopyrrole in marine bacteria by a uniquely adapted brominase-thioesterase enzyme pair. Proc Natl Acad Sci USA 2016; 113: 3797-3802.
28. Busch J, et al. Diversity and distribution of the bmp gene cluster and its Polybrominated products in the genus Pseudoalteromonas. Environ Microbiol 2019; 21: 1575-1585.
29. Alker A T, et al. Genetic examination of the marine bacterium Pseudoalteromonas luteoviolacea and effects of its metamorphosis-inducing factors. Environ Microbiol 2020; 1462-2920.15211.
30. Edmunds P J, et al. The biology of larvae from the reef coral Porites astreoides, and their response to temperature disturbances.
31. Whalen K E, Kirby C, Nicholson R M, O'Reilly M, Moore B S, Harvey E L. The chemical cue tetrabromopyrrole induces rapid cellular stress and mortality in phytoplankton. Sci Rep 2018; 8: 15498.
32. Akkipeddi S M K, et al. Halogenated compound secreted by marine bacteria halts larval urchin development. J Exp Mar Bio Ecol 2021; 538: 151540.
33. Siboni N, et al. Using bacterial extract along with differential gene expression in Acropora millepora Larvae to decouple the processes of attachment and metamorphosis. PLoS One 2012; 7: 37774.

### REFERENCES EXAMPLE 4

Alker A T, et al. 2021. Draft Genome Sequences of 10 Bacteria from the Marine Pseudoalteromonas Group. Microbiol Resour Announc 10. doi:10.1128/mra.00404-21
de Lorenzo V, Timmis K N. 1994. Analysis and construction of stable phenotypes in gram-negative bacteria with Tn5- and Tn10-derived minitransposons. Methods Enzymol 235:386-405.
Jackson S A, Fellows B J, Fineran P C. 2020. Complete Genome Sequences of the Escherichia coli Donor Strains ST18 and MFD pir. Microbiol Resour Announc 9. doi:10.1128/MRA.01014-20
Lee M E, et al. 2015. A Highly Characterized Yeast Toolkit for Modular, Multipart Assembly. doi:10.1021/sb500366v
Leonard S P, et al. 2018. Genetic Engineering of Bee Gut Microbiome Bacteria with a Toolkit for Modular Assembly of Broad-Host-Range Plasmids. ACS Synth Biol acssynbio.7b00399. doi:10.1021/acssynbio.7b00399
Ruiz-Ponte C, et al. 1998. Roseobacter gallaeciensis sp. nov., a new marine bacterium isolated from rearings and collectors of the scallop Pecten maximus. Int J Syst Bacteriol 48 Pt 2:537-542. doi:10.1099/00207713-48-2-537
Shikuma N J, et al. 2014. Marine tubeworm metamorphosis induced by arrays of bacterial phage tail-like structures. Science (80-) 343:529-533. doi:10.1126/science.1246794
Sneed J M, Sharp K H, Ritchie K B, Paul V J. 2014. The chemical cue tetrabromopyrrole from a biofilm bacterium induces settlement of multiple Caribbean corals. Proc Biol Sci 281.

### REFERENCES EXAMPLE 5

1. Prado S, et al. 2010. Review of probiotics for use in bivalve hatcheries. Vet Microbiol 145:187-197.
2. D'Alvise P W, et al. 2012. Phaeobacter gallaeciensis reduces Vibrio anguillarum in cultures of microalgae and rotifers, and prevents vibriosis in cod larvae. PLoS One 7:e43996.
3. Peixoto R S, et al. 2017. Beneficial microorganisms for corals (BMC): Proposed mechanisms for coral health and resilience. Front Microbiol 8:341.
4. Kracke F, Vassilev I, Kromer J O. 2015. Microbial electron transport and energy conservation—The foundation for optimizing bioelectrochemical systems. Front Microbiol 6:575.
5. Nozzi N E, Oliver J W K, Atsumi S. 2013. Cyanobacteria as a Platform for Biofuel Production. Front Bioeng Biotechnol 0:7.
6. Peixoto R S, et al. 2022. Harnessing the microbiome to prevent global biodiversity loss. Nat Microbiol 1-10.
7. Lozada M, Dionisi H M. 2015. Microbial Bioprospecting in Marine Environments. Springer Handb Mar Biotechnol 307-326.
8. Paulsen S S, Strube M L, Bech P K, Gram L, Sonnenschein E C. 2019. Marine Chitinolytic Pseudoalteromonas Represents an Untapped Reservoir of Bioactive Potential. mSystems 4.
9. Madsen E L. 2011. Microorganisms and their roles in fundamental biogeochemical cycles. Curr Opin Biotechnol 22:456-464.
10. McFall-Ngai M, et al. 2013. Animals in a bacterial world, a new imperative for the life sciences. Proc Natl Acad Sci USA.
11. Paoli L, et al. 2022. Biosynthetic potential of the global ocean microbiome. Nat 2022 6077917 607:111-118.
12. Sunagawa S, et al. 2015. Structure and function of the global ocean microbiome. Science (80-) 348.
13. Shetty R P, Endy D, Knight T F. 2008. Engineering BioBrick vectors from BioBrick parts. J Biol Eng 2:1-12.
14. Engler C, Kandzia R, Marillonnet S. 2008. A One Pot, One Step, Precision Cloning Method with High Throughput Capability. PLoS One 3:e3647.
15. Wiles T J, Wall E S, Schlomann B H, Hay E A, Parthasarathy R, Guillemin K. 2018. Modernized tools for streamlined genetic manipulation and comparative study of wild and diverse proteobacterial lineages. MBio 9.
16. Vasudevan R, et al. 2019. CyanoGate: A Modular Cloning Suite for Engineering Cyanobacteria Based on the Plant MoClo Syntax. Plant Physiol 180:39-55.
17. Leonard S P, et al. 2018. Genetic Engineering of Bee Gut Microbiome Bacteria with a Toolkit for Modular Assembly of Broad-Host-Range Plasmids. ACS Synth Biol 7:1279-1290.
18. Lee M E, DeLoache W C, Cervantes B, Dueber J E. 2015. A Highly Characterized Yeast Toolkit for Modular, Multipart Assembly. ACS Synth Biol 4:975-986.
19. Whitaker W R, et al. 2017. Tunable Expression Tools Enable Single-Cell Strain Distinction in the Gut Microbiome. Cell 169:538-546.e12.
20. Buijs Y, et al; 2022. Marine Proteobacteria as a source of natural products: advances in molecular tools and strategies. Nat Prod Rep 36:1333-1350.
21. Vijayan N, et al. 2019. Microbiomes of the polychaete Hydroides elegans (Polychaeta: Serpulidae) across its life-history stages. Mar Biol 166:19.
22. Bourne D G, Dennis P G, Uthicke S, Soo R M, Tyson G W, Webster N. 2013. Coral reef invertebrate microbiomes correlate with the presence of photosymbionts. ISME J 2013 77 7:1452-1458.
23. Stephens W Z, et al. 2015. The composition of the zebrafish intestinal microbial community varies across development. ISME J 2016 103 10:644-654.
24. Bourne D G, Morrow K M, Webster N S. 2016. Insights into the Coral Microbiome: Underpinning the Health and Resilience of Reef Ecosystems. Annu Rev Microbiol 70:317-340.
25. Bowman J P. 2007. Bioactive compound synthetic capacity and ecological significance of marine bacterial genus Pseudoalteromonas. Mar Drugs 5:220-241.
26. Maansson M, et al. 2016. An Integrated Metabolomic and Genomic Mining Workflow To Uncover the Biosynthetic Potential of Bacteria. mSystems 1:e00028-15.
27. Offret C, Desriac F, Le Chevalier P, Mounier J, Jegou C, Fleury Y. 2016. Spotlight on antimicrobial metabolites from the marine bacteria Pseudoalteromonas: Chemodiversity and ecological significance. Mar Drugs.
28. Chau R, Pearson L A, Cain J, Kalaitzis J A, Neilan B A. 2021. A Pseudoalteromonas Clade with Remarkable Biosynthetic Potential. Appl Environ Microbiol 87:1-16.
29. Thogersen M S, et al. 2016. Production of the bioactive compounds violacein and indolmycin is conditional in a maeA mutant of Pseudoalteromonas luteoviolacea S4054 lacking the malic enzyme. Front Microbiol 7:1-11.
30. Carpizo-Ituarte E, Hadfield M G. 1998. Stimulation of metamorphosis in the polychaete Hydroides elegans Haswell (Serpulidae). Biol Bull 194:14-24.
31. Huang S, Hadfield M G. 2003. Composition and density of bacterial biofilms determine larval settlement of the polychaete Hydroides elegans. Mar Ecol Prog Ser 260:161-172.
32. Unabia C R C, Hadfield M G. 1999. Role of bacteria in larval settlement and metamorphosis of the polychaete Hydroides elegans. Mar Biol 133:55-64.
33. Tran C, Hadfield M G. 2011. Larvae of Pocillopora damicornis (Anthozoa) settle and metamorphose in response to surface-biofilm bacteria. Mar Ecol Prog Ser 433:85-96.
34. Negri A P, et al. 2001. Metamorphosis of broadcast spawning corals in response to bacteria isolated from crustose algae. Mar Ecol Prog Ser 223:121-131.
35. Tebben J, et al. 2011. Induction of larval metamorphosis of the coral Acropora millepora by tetrabromopyrrole isolated from a Pseudoalteromonas bacterium. PLoS One 6:e19082.
36. Sneed J M, et al. 2014. The chemical cue tetrabromopyrrole from a biofilm bacterium induces settlement of multiple Caribbean corals. Proc R Soc B Biol Sci 281.
37. Cavalcanti G S, Alker A T, Delherbe N, Malter K E, Shikuma N J. 2020. The Influence of Bacteria on Animal Metamorphosis. Annu Rev Microbiol 74:137-158.
38. Shikuma N J. 2021. Bacteria-Stimulated Metamorphosis: an Ocean of Insights from Investigating a Transient Host-Microbe Interaction. mSystems 6:e00754-21.
39. Huang Y, et al. 2012. Recruitment in the sea: bacterial genes required for inducing larval settlement in a polychaete worm. Sci Rep 2.
40. Shikuma N J, et al. 2014. Marine Tubeworm Metamorphosis Induced by Arrays of Bacterial Phage Tail-Like Structures. Science (80-) 343:529-533.
41. Ericson C F, et al 2019. A contractile injection system stimulates tubeworm metamorphosis by translocating a proteinaceous effector. Elife 8:1-19.
42. Malter K E, Esmerode M, Damba M, Alker A T, Forsberg E M, Shikuma N J. 2022. Diacylglycerol, PKC and MAPK signaling initiate tubeworm metamorphosis in response to bacteria. Dev Biol 487:99-109.
43. Furste J P, et al. 1986. Molecular cloning of the plasmid RP4 primase region in a multi-host-range tacP expression vector. Gene 48:119-131.
44. Meyer R. 2009. Replication and conjugative mobilization of broad host-range IncQ plasmids. Plasmid 62:57-70.
45. Ferrieres L, et al. 2010. Silent mischief: Bacteriophage Mu insertions contaminate products of Escherichia coli random mutagenesis performed using suicidal transposon delivery plasmids mobilized by broad-host-range RP4 conjugative machinery. J Bacteriol 192:6418-6427.
46. Siebenlist U. 1979. Nuckotide sequence of the three major early promoters of bacteriophage T7. Nucleic Acids Res 6:1895-1907.
47. Jensen P R, Hammer K. 1998. The sequence of spacers between the consensus sequences modulates the strength of prokaryotic promoters. Appl Environ Microbiol 64:82-87.
48. Lee A K, Falkow S. 1998. Constitutive and Inducible Green Fluorescent Protein Expression in Bartonella henselae. Infect Immun.
49. Qi L S, Larson M H, Gilbert L A, Doudna J A, Weissman J S, Arkin A P, Lim W A. 2013. Repurposing CRISPR as an RNA-guided platform for sequence-specific control of gene expression. Cell 152:1173-1183.
50. Larson M H, et al. 2013. CRISPR interference (CRISPRi) for sequence-specific control of gene expression. Nat Protoc 8:2180-2196.
51. Balibar C J, Walsh C T. 2006. In vitro biosynthesis of violacein from L-tryptophan by the enzymes VioA-E from Chromobacterium violaceum. Biochemistry 45:15444-15457.
52. Davis J J, et al. 2020. The PATRIC Bioinformatics Resource Center: Expanding data and analysis capabilities. Nucleic Acids Res 48:D606-D612.
53. Guindon S, Gascuel O. 2003. A Simple, Fast, and Accurate Algorithm to Estimate Large Phylogenies by Maximum Likelihood. Syst Biol 52:696-704.
54. Stamatakis A, Hoover P, Rougemont J. 2008. A Rapid Bootstrap Algorithm for the RAxML Web Servers. Syst Biol 57:758-771.
55. Shikuma N J, et al. 2016. Stepwise metamorphosis of the tubeworm Hydroides elegans is mediated by a bacterial inducer and MAPK signaling. Proc Natl Acad Sci 113:10097-10102.
56. Busch J, Agarwal V, Schorn M, Machado H, Moore B S, Rouse G W, Gram L, Jensen P R. 2019. Diversity and distribution of the bmp gene cluster and its Polybrominated products in the genus Pseudoalteromonas. Environ Microbiol 21:1575-1585.
57. Agarwal V, et al. 2014. Biosynthesis of polybrominated aromatic organic compounds by marine bacteria. Nat Chem Biol 10:640-647.
58. Rocchi I, et al. 2019. A Bacterial Phage Tail-like Structure Kills Eukaryotic Cells by Injecting a Nuclease Effector. Cell Rep 28:295-301.e4.
59. Jiang F, et al 2022. N-terminal signal peptides facilitate the engineering of PVC complex as a potent protein delivery system. Sci Adv 8:2343.
60. Xu J, et al 2022. Identification and structure of an extracellular contractile injection system from the marine bacterium Algoriphagus machipongonensis.
Nat Microbiol 2022 73 7:397-410.
61. Freckelton M, Nedved B T. 2020. When does symbiosis begin? Bacterial cues necessary for metamorphosis in the marine polychaete Hydroides elegans, p. 1-15. In Cellular Dialogues in the Holobiont. CRC Press.
62. Aldred N, Nelson A. 2019. Microbiome acquisition during larval settlement of the barnacle Semibalanus balanoides. Biol Lett 15.
63. Gosselin L A, Qian P Y. 1997. Can bacterivory alone sustain larval development in the polychaete Hydroides elegans and the barnacle Balanus amphitrite? Mar Ecol Prog Ser 161:93-101.
64. Freckelton M L, Nedved B T, Hadfield M G. 2017. Induction of Invertebrate Larval Settlement; Different Bacteria, Different Mechanisms? Sci Rep 7:42557.
65. Petersen L E, et al. 2021. Photosensitivity of the Bacterial Pigment Cycloprodigiosin Enables Settlement in Coral Larvae-Light as an Understudied Environmental Factor. Front Mar Sci 8.
66. Neave M J, et al. 2016. Diversity and function of prevalent symbiotic marine bacteria in the genus Endozoicomonas. Appl Microbiol Biotechnol 100.
67. Neave M J, et al. 2017. Endozoicomonas genomes reveal functional adaptation and plasticity in bacterial strains symbiotically associated with diverse marine hosts. Sci Rep 7:1-12.
68. Pogoreutz C, et al. 2018. Dominance of Endozoicomonas bacteria throughout coral bleaching and mortality suggests structural inflexibility of the Pocillopora verrucosa microbiome. Ecol Evol 8:2240-2252.
69. Rosado P M, et al. 2019. Marine probiotics: increasing coral resistance to bleaching through microbiome manipulation. ISME J 13:921.
70. Li J, Yang Q, et al. 2022. Microbiome Engineering: A Promising Approach to Improve Coral Health. Engineering https://doi.org/10.1016/j.eng.2022.07.010.
71. Damjanovic K, et al. 2017. The contribution of microbial biotechnology to mitigating coral reef degradation. Microb Biotechnol 10:1236-1243.
72. Nyholm S V., McFall-Ngai M J. 2021. A lasting symbiosis: how the Hawaiian bobtail squid finds and keeps its bioluminescent bacterial partner. Nat Rev Microbiol 10.
73. Visick K L, Stabb E V., Ruby E G. 2021. A lasting symbiosis: how Vibrio fischeri finds a squid partner and persists within its natural host. Nat Rev Microbiol 0123456789.
74. Gould A L, Dunlap P V. 2019. Shedding Light on Specificity: Population Genomic Structure of a Symbiosis Between a Coral Reef Fish and Luminous Bacterium. Front Microbiol 10:2670.
75. Zhang X H, He X, Austin B. 2020. Vibrio harveyi: a serious pathogen of fish and invertebrates in mariculture. Mar Life Sci Technol. Springer.
76. King R K, Flick Jr G J, Pierson D, Smith S A, Boardman G D, Coale Jr C W.
2004. Identification of Bacterial Pathogens in Biofilms of Recirculating Aquaculture Systems. J Aquat Food Prod Technol 13:125-133.
77. Ushijima B, et al. 2014. Vibrio coralliilyticus strain OCN008 is an etiological agent of acute montipora white syndrome. Appl Environ Microbiol 80:2102-2109.
78. Ushijima B, et al. 2018. Factors affecting infection of corals and larval oysters by Vibrio coralliilyticus. PLoS One 13:e0199475.
79. Dittmann K K, Sonnenschein E C, Egan S, Gram L, Bentzon-Tilia M. 2019.
Impact of Phaeobacter inhibens on marine eukaryote-associated microbial communities. Environ Microbiol Rep 11:401-413.
80. Bramucci A R, Labeeuw L, Orata F D, Ryan E M, Malmstrom R R, Case R J. 2018. The bacterial symbiont Phaeobacter inhibens Shapes the life history of its algal host Emiliania huxleyi. Front Mar Sci 5:188.
81. Majzoub M E, Beyersmann P G, Simon M, Thomas T, Brinkhoff T, Egan S.
2019. Phaeobacter inhibens controls bacterial community assembly on a marine diatom. FEMS Microbiol Ecol 95:60.
82. Luo H, Moran M A. 2014. Evolutionary Ecology of the Marine Roseobacter Clade. Microbiol Mol Biol Rev 78:573-587.
83. Moran M A, et al. 2007. Ecological genomics of marine Roseobacters. Appl Environ Microbiol 73:4559-4569.
84. Tesdorpf J E, Geers A U, Strube M L, Gram L, Bentzon-Tilia M. 2022. Roseobacter Group Probiotics Exhibit Differential Killing of Fish Pathogenic Tenacibaculum Species. Appl Environ Microbiol 88.
85. Sonnenschein E C, Jimenez G, Castex M, Gram L. 2021. The Roseobacter-Group Bacterium Phaeobacter as a Safe Probiotic Solution for Aquaculture. Appl Environ Microbiol 87:1-15.
86. Dittmann K K, et al. 2020. Changes in the microbiome of mariculture feed organisms after treatment with a potentially probiotic strain of Phaeobacter inhibens. Appl Environ Microbiol 86.
87. Ruiz-Ponte C, Cilia V, Lambert C, Nicolas J L. 1998. Roseobacter gallaeciensis sp. nov., a new marine bacterium isolated from rearings and collectors of the scallop Pecten maximus. Int J Syst Bacteriol 48 Pt 2:537-542.
88. Gonzilez J M, Kiene R P, Moran M A. 1999. Transformation of sulfur compounds by an abundant lineage of marine bacteria in the alpha-subclass of the class Proteobacteria. Appl Environ Microbiol 65:3810-3819.
89. Deogaygay X, Delherbe N, Shikuma N J. 2021. Draft Genome Sequences of Two Bacteria from the Roseobacter Group. Microbiol Resour Announc 10.
90. Cavalcanti G S, et al. 2020. Complete Genome Sequences of Two Marine Biofilm Isolates, Leisingera sp. nov. Strains 201A and 204H, Novel Representatives of the Roseobacter Group. Microbiol Resour Announc 9.
91. Godwin S, et al. 2012. The Role of Coral-Associated Bacterial Communities in Australian Subtropical White Syndrome of Turbinaria mesenterina. PLoS One 7:e44243.
92. Apprill A, et al. 2009. The onset of microbial associations in the coral Pocillopora meandrina. ISME J 2009 36 3:685-699.
93. Zhang Y, et al. 2021. Shifts in abundance and network complexity of coral bacteria in response to elevated ammonium stress. Sci Total Environ 768:144631.
94. Silva D P, et al. 2021. Multi-domain probiotic consortium as an alternative to chemical remediation of oil spills at coral reefs and adjacent sites. Microbiome 9:1-19.
95. Alker A T, et al. 2022. Draft Genome Sequence of Nereida sp. Strain MMG025, Isolated from Giant Kelp. Microbiol Resour Announc 11:70-72.
96. Arahal D R, Pujalte M J, Rodrigo-Torres L. 2016. Draft genomic sequence of Nereida ignava CECT 5292T, a marine bacterium of the family Rhodobacteraceae. Stand Genomic Sci 11:1-8.
97. Ashen J B, Goff L J. 2000. Molecular and ecological evidence for species specificity and coevolution in a group of marine algal-bacterial symbioses. Appl Environ Microbiol 66:3024-3030.
98. Egan S, Harder T, Burke C, Steinberg P, Kjelleberg S, Thomas T. 2013. The seaweed holobiont: understanding seaweed-bacteria interactions. FEMS Microbiol Rev 37:462-476.
99. Singh R P, Reddy C R K. 2014. Seaweed-microbial interactions: Key functions of seaweed-associated bacteria. FEMS Microbiol Ecol 88:213-230.
100. Hofer U. 2021. A probiotic for seaweed. Nat Rev Microbiol 19:618.
101. Gibson D G, Young L, Chuang R-Y, Venter J C, Hutchison C A, Smith H O. 2009. Enzymatic assembly of DNA molecules up to several hundred kilobases. Nat Methods 6:343-345.
102. Wattam A R, et al. 2017. Improvements to PATRIC, the all-bacterial bioinformatics database and analysis resource center. Nucleic Acids Res 45:D535-D542.
103. Davis J J, et al. 2016. PATtyFams: Protein Families for the Microbial Genomes in the PATRIC Database. Front Microbiol 7:118.
104. Edgar R C. 2004. MUSCLE: Multiple sequence alignment with high accuracy and high throughput. Nucleic Acids Res 32:1792-1797.
105. Cock P J A, et al. 2009. Biopython: freely available Python tools for computational molecular biology and bioinformatics. Bioinformatics 25:1422-1423.
106. Katoh K, Standley D M. 2013. MAFFT Multiple Sequence Alignment Software Version 7: Improvements in Performance and Usability. Mol Biol Evol 30:772-780.
107. Stamatakis A. 2014. RAxML version 8: a tool for phylogenetic analysis and post-analysis of large phylogenies. Bioinformatics 30:1312-1313.
108. Nedved B T, Hadfield M G. 2009. Hydroides elegans (Annelida: Polychaeta): A Model for Biofouling Research. Mar industrial biofouling 4:203-217.
109. Nesbit K T, Shikuma N. Developmental staging of the complete life cycle of the model marine tubeworm Hydroides elegans https://doi.org/10.1101/2022.10.24.513551.
110. Alker A T, Delherbe N, Purdy T N, Moore B S, Shikuma N J. 2020. Genetic examination of the marine bacterium Pseudoalteromonas luteoviolacea and effects of its metamorphosis-inducing factors. Environ Microbiol 22:4689-4701.
111. Yang C-S, et al. 2010. Endozoicomonas montiporae sp. nov., isolated from the encrusting pore coral Montipora aequituberculata. Int J Syst Evol Microbiol 60:1158-1162.
112. de Lorenzo V, Timmis K N. 1994. Analysis and construction of stable phenotypes in gram-negative bacteria with Tn5- and Tn10-derived minitransposons. Methods Enzymol 235:386-405.
113. Simon R, Priefer U, Pühler A. 1983. A Broad Host Range Mobilization System for In Vivo Genetic Engineering: Transposon Mutagenesis in Gram Negative Bacteria. Bio/Technology 1983 19 1:784-791.

A number of embodiments of the invention have been described. Nevertheless, it can be understood that various modifications may be made without departing from the spirit and scope of the invention. Accordingly, other embodiments are within the scope of the following claims.

1: A transposon vector comprising:
(a) a nucleic acid sequence as set forth in a nucleic acid sequence comprising a sequence as set forth in SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11 or SEQ ID NO:12, and
(b) a plurality of modular elements operatively linked to each other, wherein the plurality of modular elements comprise:
a Type-2 broad host range CP25 promoter,
a Type-3 GFP or mRuby protein coding sequence (CDS) or structural gene (or a protein coding sequence),
a Type-4 terminator,
a Type-1 and a Type-5 connector, and
a Type-8 RSF1010 backbone.

2: The transposon vector of claim 1, wherein the vector comprises a modular assembly comprising:
(a) 5′-Type1 (linker)-Type2 (Bmp1p, macBp, or CP25p)-Type3 (gfp, mRuby, or nanoluc)-Type4 (T7-terminator)-Type5 (linker)-Type6-7-Type8 (Tn7/Tn10/RSF1010)-3′;
(b) a Type2-CP25p or Type8-Tn7 vector comprising:
5′-Type1 (linker)-Type2 (CP25p)-Type3 (gfp)-Type4 (T7-terminator)-Type5 (linker)-Type6-7-Type8 (Tn7)-3′,
5′-Type1 (linker)-Type2 (CP25p)-Type3 (mRuby)-Type4 (T7-terminator)-Type5 (linker)-Type6-7-Type8 (Tn7)-3′, or
5′-Type1 (linker)-Type2 (CP25p)-Type3 (nanoluc)-Type4 (T7-terminator)-Type5 (linker)-Type6-7-Type8 (Tn7)-3′;
(b) a Type2-CP25p or Type8-Tn10 vector comprising:
5′-Type1 (linker)-Type2 (CP25p)-Type3 (gfp)-Type4 (T7-terminator)-Type5 (linker)-Type6-7-Type8 (Tn10)-3′,
5′-Type1 (linker)-Type2 (CP25p)-Type3 (mRuby)-Type4 (T7-terminator)-Type5 (linker)-Type6-7-Type8 (Tn10)-3′, or
5′-Type1 (linker)-Type2 (CP25p)-Type3 (nanoluc)-Type4 (T7-terminator)-Type5 (linker)-Type6-7-Type8 (Tn10)-3′;
(c) a Type2-CP25p or a Type8-RSF1010 vector comprising:
5′-Type1 (linker)-Type2 (CP25p)-Type3 (gfp)-Type4 (T7-terminator)-Type5 (linker)-Type6-7-Type8 (RSF1010)-3′,
5′-Type1 (linker)-Type2 (CP25p)-Type3 (mRuby)-Type4 (T7-terminator)-Type5 (linker)-Type6-7-Type8 (RSF1010)-3′, or
5′-Type1 (linker)-Type2 (CP25p)-Type3 (nanoluc)-Type4 (T7-terminator)-Type5 (linker)-Type6-7-Type8 (RSF1010)-3′;
(d) a Type2-Bmp1p or a Type8-Tn7 vector comprising:
5′-Type1 (linker)-Type2 (Bmp1p)-Type3 (gfp)-Type4 (T7-terminator)-Type5 (linker)-Type6-7-Type8 (Tn7)-3′,
5′-Type1 (linker)-Type2 (Bmp1p)-Type3 (mRuby)-Type4 (T7-terminator)-Type5 (linker)-Type6-7-Type8 (Tn7)-3′, or
5′-Type1 (linker)-Type2 (Bmp1p)-Type3 (nanoluc)-Type4 (T7-terminator)-Type5 (linker)-Type6-7-Type8 (Tn7)-3′;
(e) a Type2-Bmp1p or Type8-Tn10 vector comprising:
5′-Type1 (linker)-Type2 (Bmp1p)-Type3 (gfp)-Type4 (T7-terminator)-Type5 (linker)-Type6-7-Type8 (Tn10)-3′,
5′-Type1 (linker)-Type2 (Bmp1p)-Type3 (mRuby)-Type4 (T7-terminator)-Type5 (linker)-Type6-7-Type8 (Tn10)-3′, or
5′-Type1 (linker)-Type2 (Bmp1p)-Type3 (nanoluc)-Type4 (T7-terminator)-Type5 (linker)-Type6-7-Type8 (Tn10)-3′;
(f) a Type2-Bmp1p or a Type8-RSF1010 vector comprising:
5′-Type1 (linker)-Type2 (Bmp1p)-Type3 (gfp)-Type4 (T7-terminator)-Type5 (linker)-Type6-7-Type8 (RSF1010)-3′,
5′-Type1 (linker)-Type2 (Bmp1p)-Type3 (mRuby)-Type4 (T7-terminator)-Type5 (linker)-Type6-7-Type8 (RSF1010)-3′, or
5′-Type1 (linker)-Type2 (Bmp1p)-Type3 (nanoluc)-Type4 (T7-terminator)-Type5 (linker)-Type6-7-Type8 (RSF1010)-3′
(g) a Type2-MacBp or a Type8-Tn7 vector comprising:
5′-Type1 (linker)-Type2 (MacBp)-Type3 (gfp)-Type4 (T7-terminator)-Type5 (linker)-Type6-7-Type8 (Tn7)-3′,
5′-Type1 (linker)-Type2 (MacBp)-Type3 (mRuby)-Type4 (T7-terminator)-Type5 (linker)-Type6-7-Type8 (Tn7)-3′, or
5′-Type1 (linker)-Type2 (MacBp)-Type3 (nanoluc)-Type4 (T7-terminator)-Type5 (linker)-Type6-7-Type8 (Tn7)-3′;
(h) a Type2-MacBp or a Type8-Tn10 vector comprising:
5′-Type1 (linker)-Type2 (MacBp)-Type3 (gfp)-Type4 (T7-terminator)-Type5 (linker)-Type6-7-Type8 (Tn10)-3′,
5′-Type1 (linker)-Type2 (MacBp)-Type3 (mRuby)-Type4 (T7-terminator)-Type5 (linker)-Type6-7-Type8 (Tn10)-3′, or
5′-Type1 (linker)-Type2 (MacBp)-Type3 (nanoluc)-Type4 (T7-terminator)-Type5 (linker)-Type6-7-Type8 (Tn10)-3′; or
(i) a Type2-MacBp or Type8-RSF1010 vector comprising:
5′-Type1 (linker)-Type2 (MacBp)-Type3 (gfp)-Type4 (T7-terminator)-Type5 (linker)-Type6-7-Type8 (RSF1010)-3′,
5′-Type1 (linker)-Type2 (MacBp)-Type3 (mRuby)-Type4 (T7-terminator)-Type5 (linker)-Type6-7-Type8 (RSF1010)-3′, or
5′-Type1 (linker)-Type2 (MacBp)-Type3 (nanoluc)-Type4 (T7-terminator)-Type5 (linker)-Type6-7-Type8 (RSF1010)-3′.

3: The transposon vector of claim 1, wherein the vector comprises a modular assembly and a sequence selected from the group consisting of: a Type-1 linker vector (SEQ ID NO:3); a Type-2 CP25 promoter vector (SEQ ID NO:4); a Type-2 macB promoter vector (SEQ ID NO:5; Type-2 bmp1 promoter vector (SEQ ID NO:6); Type-3 coding GFP vector (SEQ ID NO:7); Type-3 coding nanoluc vector (SEQ ID NO:8); Type-3 coding mRuby vector (SEQ ID NO:9); Type-4 terminator vector (SEQ ID NO:10); Type-5 linker vector (SEQ ID NO:11) and Type-8 RSF1010 backbone vector (SEQ ID NO:12).

4: A cell comprising or having contained therein a transposon vector comprising a nucleic acid sequence as set forth in SEQ ID NO:1 or SEQ ID NO:2.

5: The cell of claim 4, wherein the cell is a bacterial cell.

6: A method for genetically modifying a cell comprising inserting into the cell a transposon vector as set forth in claim 1.

7: A kit or a product of manufacture comprising a transposon vector as set forth in claim 1.

8: A method for enhancing growth of a coral or enhancing growth or a coral reef, or enhancing or stimulating coral metamorphosis, comprising exposing the coral or coral reef to a genetically engineered marine bacterial cell (bacterium) that expresses and/or secretes into the extracellular milieu higher than wild type levels of tetrabromopyrrole (TBP).

9-10. (canceled)

11: The transposon vector of claim 1, wherein the transposon vector comprises or has contained therein a structural gene or protein coding sequence.

12: The transposon vector of claim 11, wherein the structural gene or protein coding sequence is selected from the group consisting of: a gene encoding or protein coding sequence for tetrabromopyrrole (TBP), bmp (2,2-Bis(bromomethyl)-1,3-propanediol) genes, metamorphosis associated contractile structures gene B, metamorphosis associated contractile structures gene S, metamorphosis associated contractile structures gene R, LPS (lipopolysaccharide) genes, EPS (extracellular polymeric substances) genes, OMV (outer membrane vesicle) genes, omp (outer membrane protein) genes, RNA polymerase sigma factor rpoS, RNA polymerase sigma factor rpoE, CRISPR Cas9 variants, CRISPR variants, and a tag.

13: The transposon vector of claim 12, wherein the tag is or comprises: FLAG, histidine or polyhistidine (His), Sumo, or GST (glutathione S transferase).

14: The cell of claim 5, wherein the bacterial cell is a marine bacterium.

15: The cell of claim 14, wherein the marine bacterium of the genus or family Nereida, Roseobacter, Pseudoalteromonas and/or Vibrio, optionally Pseudoalteromonas sp. PS5.

16: The cell of claim 6, wherein the cell is a bacterial cell.

17: The cell of claim 16, wherein the bacterial cell is a marine bacterium.

18: The cell of claim 16, wherein the marine bacterium is the genus or family Nereida, Roseobacter, Pseudoalteromonas and/or Vibrio, optionally Pseudoalteromonas sp. PS5.

19: The method of claim 8, wherein the genetically engineered marine bacterium is of the genus or family Nereida, Roseobacter, Pseudoalteromonas and/or Vibrio, optionally Pseudoalteromonas sp. PS5.

20: The method of claim 8, wherein the genetically engineered marine bacterium comprises or has contained therein a transposon vector as set forth in claim 1.

21: A cell comprising or having contained therein a transposon vector as set forth in claim 1.

22: The cell of claim 21, wherein the cell is a bacterial cell.
